(19) United States
(12) Patent Application Publication
(10) Pub. No.: US 2003/0236393 A1
(43) Pub. Date: Dec. 25, 2003

(54) Virulence genes of M. marinum and M. tuberculosis

(75) Inventor: Michele Trucksis, Worcester, MA (US)

Correspondence Address: MILLEN, WHITE, ZELANO & BRANIGAN, P.C., 2200 CLARENDON BLVD., SUITE 1400, ARLINGTON, VA, 22201, US

(73) Assignees: UNITED STATES OF AMERICA DEPT OF VETRANS AFFAIRS, Washington, DC (US); University of Maryland, Baltimore, MD (US)

(21) Appl. No.: 10/394,575

(22) Filed: Mar. 24, 2003

Related U.S. Application Data
(60) Provisional application No. 60/366,262, filed on Mar. 22, 2002.

(51) Int. Cl. C07H 21/02; C07H 21/04

(52) U.S. Cl. 536/23.1

(57) ABSTRACT

Methods for identifying, isolating and mutagenizing virulence genes of mycobacteria, e.g., M. marinum and M. tuberculosis, are described. Also described are isolated virulence genes and fragments of them, isolated gene products and fragments of them, avirulent bacteria in which one or more virulence genes are mutagenized, attenuated vaccines containing such mutant bacteria, and methods to elicit an immune response in a host, using such mutant bacteria.

This application claims the benefit of the filing date of U.S. Provisional Application Serial No. 60/367,206 filed Mar. 26, 2002 and No. 60/366,262 filed Mar. 22, 2002, the entire disclosures of which are hereby incorporated by reference herein. The PCT, WO01/19993, is incorporated by reference herein in its entirety.

### BACKGROUND OF THE INVENTION

Mycobacteria are bacterial organisms which are implicated in diseases such as, e.g., tuberculosis. It would be desirable to provide means for treating or preventing conditions caused by such mycobacteria, e.g., by immunization.

### DESCRIPTION OF THE INVENTION

This invention relates, e.g., to virulence genes of mycobacteria. The invention provides methods to identify and isolate virulence genes of, for example, Mycobacterium marinum, a fish bacterium, and Mycobacterium tuberculosis, the primary etiologic agent of human tuberculosis. The invention also provides methods to mutagenize such virulence genes, thereby allowing the generation and isolation of avirulent mycobacteria. The invention also relates to isolated virulence genes and variants and fragments thereof; to isolated virulence gene products and variants and fragments thereof; to mutant, avirulent, bacteria; to attenuated vaccines comprising the mutant bacteria; and to methods to elicit an immune response in a host, using such mutant bacteria.

One embodiment of the invention is a method for identifying a virulence gene of M. marinum, comprising

a) mutagenizing M. marinum bacteria by introducing into said bacteria a plasmid which comprises a tagged (e.g., signature-tagged) transposon, whereby the transposon integrates into and disrupts a gene in the bacteria,

b) introducing said mutagenized bacteria into a host susceptible to infection thereof (e.g., a goldfish),

c) identifying a mutagenized bacterium which comprises a tagged transposon and which exhibits reduced viability in the host, compared to other mutagenized or (non-mutagenized) M. marinum bacteria,

d) cloning and/or sequencing (characterizing) a nucleic acid sequence which flanks the integrated transposon in said identified mutagenized bacterium, and

e) identifying a wild type M. marinum gene which comprises at least a portion of said flanking sequence.

Of course, the above method can be carried out using one or more of the steps, in any order, effective to achieve the intended purpose.

Another embodiment is a method for identifying a virulence gene of M. tuberculosis, comprising identifying an M. marinum virulence gene as described above, and further comprising,

comparing said flanking nucleic acid sequence to a databank of M. tuberculosisnucleic acid sequences, and/or comparing the sequences of peptides which are coded for by said flanking sequences to a known M. tuberculosis protein database, and

identifying an M. tuberculosis gene which comprises a sequence that is substantially identical to said flanking sequences and/or polypeptides encoded by them. In other embodiments, the degree of identity can be less than substantially identical, e.g., about 35-50%, or about 50-70%, or about 70-90%.

Another embodiment is a method for isolating a mutagenized M. marinum bacterium which exhibits reduced virulence in a host susceptible to infection thereof compared to a non-mutagenized M. marinum bacterium, comprising integrating a tagged (e.g., signature-tagged) transposon into the DNA of a M. marinum bacterium in a manner effective to produce reduced virulence, and isolating said mutagenized bacterium.

Another embodiment is an avirulent M. marinum bacterium in which one or more genes comprising a nucleic acid of SEQ ID NOs: 4, 13, 23, 25, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117 are mutated, thereby rendering the M. marinum bacterium less virulent. In another embodiment, genes comprising a sequence of SEQ ID NOs: 117, 51, 53, 57 or 61 are so mutated. In another embodiment, genes comprising a nucleic acid of SEQ ID NOs: 59, 67, 71, 73, 75, 79, 81, 83, 85, 87, 89, 91, 93, 95, 97, 99, 101, 103, 105, 107 or 109 are so mutated. Combinations of the mutations of the invention are also encompassed by the invention. In a preferred embodiment, the mutations are large deletions within those sequences, e.g. about >50%, >75%, >90%, >95%, or about 100% of the coding sequence of the gene(s). In another embodiment, genes comprising a sequence of SEQ ID NOs: 115, 117, 111, 57, 55, 65, 51, 53, 63, 69, or 77 are mutated, e.g. so as to delete at least about 50%, e.g., at least about 90%, of the coding sequence, thereby rendering the M. marinum bacterium less virulent, wherein the remaining coding sequence is not any portion of the oligonucleotide sequence identified as being a flanking sequence of mutants 41.2, 86.1, 60.2, 80.1, 62.2, 80.8, 32.2, 42.2, 68.6, 114.7, or 95.3 in WO01/19993, which is incorporated by reference herein in its entirety.

Another embodiment is a pharmaceutical composition or an attenuated vaccine comprising an avirulent M. marinum bacterium of the invention and a pharmaceutically acceptable carrier.

Another embodiment is an avirulent M. tuberculosis bacterium in which one or more virulence genes identified as described above are mutated, e.g., so as to delete at least about 50% (e.g., at least about 90%) of the coding sequence, to render the M. tuberculosis bacterium less virulent. Another embodiment is an avirulent M. tuberculosis bacterium in which one or more of the virulence genes Rv0822c, Rv3137, Rv2348c, Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c, Rv1662, Rv1661, Rv2947c, Rv0405, Rv2934, Rv2931, Rv2932, Rv3825c, Rv1527c, Rv2933, Rv1664, Rv3800c, Rv1180 or Rv2935, are so mutated. Another embodiment is an avirulent M. tuberculosis bacterium in which one or more of the virulence genes Rv0101, Rv1285, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0060c, Rv0355c, Rv0213c, AE006933, Rv0449c, Rv0797, Rv3106, or Rv3347c are so mutated. In another embodiment, virulence genes Rv2048c, Rv1662, Rv1661, Rv0159c, Rv0160c, Rv0305c, Rv0355c, Rv0304c, Rv3347c, Rv1918c, Rv1753c, Rv1984c, Rv3452, Rv3451, Rv2947c, Rv0405, Rv2934, Rv2931, Rv2932, Rv3825c, Rv1527c, Rv2933, Rv1664, Rv3800c, Rv1180 or Rv2935 are so mutated. Combinations of the above mutations are also encompassed by the invention. In a preferred embodiment, the mutations are large deletions within those sequences, e.g., about >50%, >75%, >90%, >95%, or about 100% of the coding sequence of the gene(s). Another embodiment is a pharmaceutical composition or an attenuated vaccine comprising one or more of the above avirulent M. tuberculosis bacteria (e.g., an M. tuberculosis strain constructed with one or more mutations in one or more of the above virulence genes) and a pharmaceutically acceptable carrier.

Another embodiment is an isolated nucleic acid (polynucleotide) of M. marinum comprising the sequence of SEQID NOs: 4, 13, 23, 25, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, or 117, or a variant or fragment thereof. Another embodiment is a nucleic acid which is complementary to at least a portion of said isolated M. marinum nucleic acid, or which can hybridize to at least a portion of said isolated M. marinum nucleic acid under selected (e.g., high) stringency conditions. In other embodiments, the isolated M. marinum nucleic acid comprises a gene; or the isolated M. marinum nucleic acid or fragments thereof are cloned into, and/or expressed in, an expression vector.

Another embodiment is an isolated nucleic acid of M. tuberculosis, comprising a virulence gene identified as above, or a variant or fragment thereof. Another embodiment is a nucleic acid which is complementary to at least a portion of said isolated M. tuberculosis nucleic acid, or which can hybridize to at least a portion of said isolated M. tuberculosis nucleic acid under selected (e.g., high) stringency conditions. In other embodiments, the isolated M. tuberculosis nucleic acid or fragments thereof are cloned into, and/or expressed in, an expression vector.

Another embodiment is a method for generating an avirulent M. marinum or M. tuberculosis bacterium, comprising mutagenizing a nucleic acid and/or gene of the invention, so as to delete at least greater than about 50%, e.g., at least about >75%, >90%, >95% or 100% of the coding sequence(s).

Another embodiment is a method to elicit an immune response in a fish, comprising introducing into the fish an avirulent M. marinum bacterium made (e.g., isolated, constructed) as described above. Another embodiment is a method to elicit an immune response in a human or non-human animal (e.g., domestic or farm animal, such as a cow) host, comprising introducing into said host an avirulent, M. tuberculosis bacterium, in which one or more virulence genes of the invention are mutated.

Another embodiment is a method to identify an agent which reduces the ability of an M. marinum or M. tuberculosis bacterium to survive in a host, comprising disrupting expression of one of the M. marinum or M. tuberculosis genes of the invention.

Another embodiment is a method to test for the presence of an M. marinum or M. tuberculosis infection in a subject (e.g., in a human), comprising administering to the subject one or more proteins encoded by one or more nucleic acids and/or gene of the invention, and determining if cell-mediated immunity is induced.

A wide variety of Mycobacteria species can be used in the invention. In a most preferred embodiment, the bacterium is Mycobacterium marinum (M. marinum), which causes fish tuberculosis, as well as, in humans, skin infection or localized nodular and ulcerated lesions (mariner's tuberculosis) on the extremities and, in immunocompromised patients, systemic disease; Mycobacterium tuberculosis (M. tuberculosis), the primary etiologic agent for tuberculosis (TB) in man; or Mycobacterium bovis (M. bovis), which causes human or bovine tuberculosis. Other species of Mycobacterium which can be used in the invention include, e.g., M. bovis BCG, M. africanum, M. leprae, M. microti, M. smegmatis, M. vaccae, M. ulcerans, M. haemophilum, M. fortuitum, M. chelonae, and others.

The term “virulent” in the context of mycobacteria refers to a bacterium or strain of bacteria that replicates within a host cell or animal within the mycobacterium host range at a rate which is detrimental to the cell or animal, or that induces a host response which is detrimental. More particularly, virulent mycobacteria persist longer in a host than avirulent bacteria. Virulent mycobateria are typically disease producing; and infection leads to various disease states including fulminant disease in the lung, disseminated systemic milliary tuberculosis, tuberculosis meningitis, and/or tuberculosis abscesses of various tissues. Infection by virulent mycobacteria often results in death of the host organism.

By contrast, the term “avirulent,” as used herein, refers to a bacterium or strain of bacteria that does not replicate within a host cell or animal within its host range; replicates at a rate which is not significantly detrimental to the cell or animal; and/or does not induce a detrimental host response. An avirulent (e.g., attenuated, non-pathogenic) strain is incapable of inducing a full suite of symptoms of the disease that is normally associated with its virulent pathogenic counterpart. Avirulent bacteria exhibit a reduced ability, or an inability, to survive in a host, but not all bacteria which exhibit such an impaired ability to survive in a host are avirulent. For example, in a simultaneous in vivo test of several mutant bacteria, certain mutants which are unable to compete with other mutants may not, when tested in the presence of the other strains, replicate efficiently or survive in the host; however, such bacteria, when tested individually, may prove to be virulent. An avirulent bacterium can contain one or more mutations in one or more virulence genes.

A “virulence gene” encodes a gene product (“virulence factor, virulence determinant”) which contributes, directly or indirectly, to infection (e.g., attachment, invasion, transport into the cell, replication, etc.) and/or to tissue destruction and/or disease. A virulence gene can code for or modify, e.g., an adhesion molecule or other molecule which aids in the attachment to or invasion of a host cell; a toxin (e.g., a secreted factor which can cause lysis or damage of a host cell—for example, a small molecule such as a polyketide, or an enzyme such as a phospholipase, lipase, esterase or protease); a factor required for efficient secretion of such a toxin; a factor involved in intracellular multiplication or growth; a factor involved in resistance to host defenses; a factor which can stimulate a host cell to produce an inflammatory product or cytokine that can amplify tissue damage in a host; or a factor which regulates the production and/or activity of a virulence factor. Also included are certain functions which resemble “housekeeping” functions, e.g., functions which allow bacteria to provide nutrients that are limiting in a host, such as factors which aid in the acquisition of iron, or certain enzymes of purine or pyrimidine biosynthesis. For a review of some of the putative or suspected virulence determinants of Mycobacterium tuberculosis, see Quinn et al (1996). Curr. Top. Microbiol. Immunol. 215, 131-156.

By a “host” for a bacterium is meant an organism, or a cell or tissue of an organism, which can be infected by the bacterium and which exhibits consequences of that infection. For example, Mycobacterium marinum can infect and cause symptoms in the frog (Rana pipiens) or in any of about 150 fresh-water or salt-water species of fish. In an especially preferred embodiment, the host for Mycobacterium marinum is the goldfish, Carassius auratus. Well-established animal models for M. tuberculosis include, e.g., guinea pig, mouse, rabbit and monkey; and many natural hosts exist for that bacterium, including large animals such as the elephant. Many other bacteria/host combinations are possible. See, e.g., B. Bloom, ed., (1994). Tuberculosis: Pathogenesis, Protection, and Control, ASM Press, Washington, D.C. Chapter 11, for a discussion of tuberculosis in wild and domestic animals.

A system in which goldfish are infected by M. marinum (the “goldfish model”) offers a number of advantages for experimental studies. For example, M. marinum has a generation time of only 4 hours (as compared, e.g., to the greater than 20 hour generation time of M. tuberculosis), and studies with M. marinum can be carried out in a Biosafety Level 2 facility (whereas a Biosafety Level 3 facility is required, e.g., for studies with M. tuberculosis). M. marinum can serve as an appropriate surrogate model for the study of M. tuberculosis. M. marinum and the M. tuberculosis complex have been shown to be closely related by, e.g., DNA hybridization and 16S rRNA gene sequence analysis (see, e.g., Tønjum et al (1998). J. of Clinical Microbiology 36, 918-925). The disease progression and symptoms of fish infected with M. marinum mimic those of humans infected with M. tuberculosis: in both types of hosts, organs in all parts of the body can be infected; both bacteria replicate within macrophages and reside in an endosomal compartment which is nonacidic and does not fuse with the lysosomal compartment; and both bacteria may replicate in macrophages, eventually killing the macrophages.

Examples 1B and 1C show, e.g., that the pathology in the goldfish model parallels that of human tuberculosis. Depending on the dose of M. marinum organisms which is inoculated into a fish, acute or chronic disease is elicited. The pathology of the acute disease includes severe peritonitis and necrosis with all animals dying within 17 days of infection. The pathology of the chronic disease includes progressive granuloma formation. Granulomas with different histopathological features (necrotizing, non-necrotizing and caseous) are seen in the experimentally infected goldfish, which is consistent with the granuloma types seen in naturally infected animals and parallels the types of granulomas found in human tuberculosis. Isolation of M. marinum from fish tissue is possible throughout the course of the experiment presented in Example 1 (up to 16 weeks) indicating, as in human tuberculosis, the persistence of the organisms in the host. Example 2 shows that the goldfish model can be used to distinguish virulent and avirulent forms of M. marinum. Further disclosure of how to make the goldfish model, and how to use it, e.g., to characterize molecular pathogenesis, can be found, e.g., in Talaat A. M. et al (1998). Infection and Immunity 66, 2938-2942.

As an initial step in isolating virulence mutants, bacteria, e.g., M. marinum, can be mutated by any of a variety of routine procedures which are well-known in the art, e.g., exposure to chemical agents, irradiation, genetic engineering, transposon mutagenesis, or the like. As used in this application, the term a “mutation” means any change (in comparison with the appropriate parental strain) in the DNA sequence of an organism, e.g., a single (or multiple) base change, insertion, deletion, inversion, translocation, duplication, or the like. A mutation can be polar or non-polar, a frameshift or in phase. Preferably, in particular when a mutated bacterium is used as part of a treatment regimen or a vaccine, the mutation is substantially incapable of reverting to the wild type.

In a most preferred embodiment, mutagenesis is carried out by a transposon mutagenesis system that carries sequence-specific tags, sometimes known as signature-tagged mutagenesis (STM). The unique tag sequence allows differentiation of individual mutants among an inoculum pool of mutants. The STM protocol permits the screening of a large number of mutants using a small number of animals. This method was developed by Hensel et al (Hensel et al (1995). Science 269, 400-403; U.S. Pat. No. 5,876,931 to Holden). Variations of the method and procedures for using it to isolate bacterial virulence mutants are also disclosed in, e.g., Shea et al (1996). Proc. Natl. Acad. Sci. 93, 2593-2597; Mei et al (1997). Mol. Microbiol. 26, 399-407; Schwan et al (1998). Infec. Immun. 66, 567-572; and Chiang et al (1998). Mol. Microbiol. 27, 797-805. Example 3 shows the use of the STM system for the mutagenesis of M. marinum.

Any of a variety of methods can be used to generate a bank of plasmids carrying unique signature-tagged transposons. A most preferred embodiment is shown in Example 3A. Here, 96 independent, non-cross-hybridizing, signature-tagged transposons, each of which is hybridization- and amplification-efficient, are cloned into a mycobacteria suicide vector which carries a selectable marker. Many variants of such vectors, carrying any of a variety of selectable markers, can be used, of course. In example 3A, the marker is a kanamycin-resistance gene.

To generate a mutant mycobacterium library, plasmids from a master plasmid collection are introduced individually (e.g., separately) into mycobacteria, preferably M. marinum, by any of a variety of routine, art-recognized techniques (e.g., phage transduction, shooting a “gene gun,” electroporation, or other conventional techniques). In a most preferred embodiment, as shown in Example 3C, plasmids are introduced into M. marinum by electroporation. Any desired number of transformed bacteria can be selected from each transformation. In Example 3C, ninety-six transformations are performed, one with each of the 96 master plasmids; and ten independent transformants are selected from each transformation, to yield a library of 960 transformants. As Example 3B shows, the transposons integrate randomly into the M. marinum chromosome. In the ideal circumstance, each integrated transposon disrupts a different gene, or a different portion thereof, to create a library of, in this example, 960 differently mutagenized bacteria.

Pools of mutagenized bacteria, each of which can be detected independently by virtue of its unique signature tag, are introduced into an appropriate host, e.g., a goldfish (an “input pool”). Bacteria may be introduced into an animal by any route, e.g., orally, intraperitoneally, intravenously or intranasally; for fish, the preferred routes of administration are oral or, most preferably, intraperitoneal. It may be useful to compare, e.g., virulence genes identified by oral administration to those identified by intraperitoneal administration, as some genes may be required to establish infection by one route but not by the other. Bacteria are left in the host for a suitable length of time, which is a function of both the microorganism and the host. A method for optimization of some of the infection parameters for the M. marinum/goldfish system is shown, e.g., in Examples 1 and 2.

Assays are performed to determine whether the bacteria are able to survive in the host during the period of infection. Any of a variety of such assays can be used, e.g., subtractive hybridization, differential display, or the like. In a most preferred embodiment, as shown in Example 4A, after an optimized period of infection by a pool of M. marinum mutants, fish are sacrificed and one or more internal organs, e.g., spleen, liver, kidney, peritoneum, heart, pancreas, or other organs evident to one of skill in the art, are cultured to isolate the mutant bacteria which were able to survive in the fish, defined as the output pool. A hybridization protocol to identify mutants present in the input and output pools is described in Example 4A. Mutants which are present in the input pool, but which cannot be detected after a predetermined time of infection has elapsed in the output pool, are candidates for avirulent mutants, i.e., mutants which are unable to infect, replicate and/or cause damage, in a particular cell type or tissue.

In order to confirm that an M. marinum mutant is avirulent, each putative virulence mutant can be re-examined individually, e.g., in the goldfish model. In a preferred embodiment, the median survival time (MST) of goldfish infected with a lethal dose (about 5×10⁸ cfu) of a putative virulence mutant can be determined, and those mutants which allow goldfish to survive longer than fish inoculated with an equivalent dose of wild type organisms are categorized as putative virulence mutants. Many other types of screening assays can be used, including Competitive Indices, histopathology examinations of one or more of the organs described above, colony counts in organ homogenates, and analysis of the ability of a mutant to induce granuloma formation. Representative protocols for each of these methods are described, e.g., in Example 4B. In addition to confirming the existence of a virulence mutant, data collected on each mutant can yield clues to the pathogenesis pathways of M. marinum in the goldfish model. Methods to show that Koch's postulates have been fulfilled (proving that a postulated virulence gene is responsible for disease symptoms) are routine; one such method is presented in Example 8.

Alternative approaches to the STM technique can be used to identify avirulent M. marinum mutants. For example, one can screen a library of M. marinum cosmids in M. smegmatis. In the goldfish model, M. smegmatis does not persist in tissue when inoculated at a dose of 10⁷ organisms/fish. This is in contrast to M. marinum, which can be isolated from fish tissue throughout the course of a 56 day experiment. In this alternative approach, one can inject the fish with pools of the M. marinum cosmids in M. smegmatis and look for those which survive in the animal. A library of M. marinum cosmids in M. smegmatis can be obtained routinely, using standard, art-recognized procedures.

Once an insertionally mutated M. marinum bacterium has been identified as being a (putative) virulence mutant, a wild type M. marinum can be engineered to contain a more well-defined (e.g., non-polar) mutation. The introduction of such a well-defined mutation into a new genetic background can confirm that the original phenotype was the result of the transposition event, rather than a secondary mutation. Furthermore, a well-defined mutation can be used to ascertain the presence, if any, of polarity effects. For example, the insertion of a transposon into a gene which is part of an operon can have polar effects on downstream genes in the operon. One method to determine if a given defect results from inactivation of the gene into which a transposon integrated, or if the actual virulence gene(s) lies downstream of the integration site, is to generate a small, in-frame, non-polar, deletion or insertion into a wild type correlate of the gene into which the transposon had integrated. If such a mutant, when tested, for example as described above in the fish model, does not exhibit an avirulent phenotype, other genes in the operon can be mutated and analyzed in the same manner until one (or more) virulence genes are identified. That is, nucleic acid sequences which flank the integrated transposon can be cloned and sequenced in several sequential steps (e.g., one can “walk” down an operon) until a virulence gene is identified. Of course, the invention includes genes which lie downstream of a gene in which a polar mutation results in an avirulent phenotype. Such genes can be considered to be “genes of the invention” or “genes identified by methods of the invention.”

As a first step in performing site-specific mutagenesis of a gene of interest, it is preferable to isolate (e.g., clone) at least a portion of the corresponding wild type gene. If the gene is part of an operon, some, if not all, of the other genes in the operon can also be isolated. As used in this application, the term “isolated” (referring, e.g., to a gene or gene product, nucleic acid, protein, bacterium, etc.) means being in a non-naturally-occurring form. Methods to clone genes, particularly those containing a unique marker, are routine for one of ordinary skill in the art. (See, e.g., Sambrook, J. et al (1989). Molecular Cloning, a Laboratory Manual. Cold Spring Harbor Laboratory Press, Cold Spring Harbor, N.Y.; Ausubel, F. M. et al (1995). Current Protocols in Molecular Biology, N.Y., John Wiley & Sons; Davis et al. (1986), Basic Methods in Molecular Biology, Elsevir Sciences Publishing, Inc., New York; Hames et al. (1985), Nucleic Acid Hybridization, IL Press; Dracopoli, N. C. et al., Current Protocols in Human Genetics, John Wiley & Sons, Inc.; and Coligan, J. E. et al., Current Protocols in Protein Science, John Wiley & Sons, Inc for many of the molecular biology techniques referred to in this application, including isolating, cloning, modifying, labeling, manipulating, sequencing, and otherwise treating or analyzing nucleic acid and/or protein.). In one method, clones comprising a gene(s) of interest can readily be identified and isolated from a wild type library (e.g., a cosmid library, Bacterial Artificial Chromosome (BAC) library (Brosch, R. et al (1998). Infect. Immun. 66, 2221-2229; Philipp, W. J. et al (1996). PNAS 93, 3132-37), phage library, cDNA library, or the like), using conventional, routine, procedures in the art. Methods for subcloning a gene(s) of interest are also routine for one of ordinary skill in the art.

Example 6 describes a preferred embodiment of the invention, in which a hybridization probe corresponding to gene sequences flanking the site of transposon integration in an M. marinum mutant is used to screen a cosmid library of wild type M. marinum genes. Because many M. marinum genes are about 2 kb in size, and the average DNA insert in a cosmid library can be about 30-40 kb, it is likely that a cosmid clone so identified will contain the entire operon, if any, in which the gene of interest is located. It is understood, of course, that the genes and clones referred to in this application typically are double-stranded; therefore, a probe “corresponding to” a given sequence can be designed to hybridize to either of the strands of the DNA duplex, or to a nucleic acid (e.g., RNA or cDNA) which is complementary to one strand of the duplex.

The term “a cloned gene,” as used herein, can encompass (but not necessarily does) not only the regions of DNA that code for a polypeptide but also regulatory regions of DNA such as regions of DNA that regulate transcription, translation and, for some microorganisms, splicing of RNA. Thus, a “gene” can include promoters, transcription terminators, ribosome-binding sequences and, for some organisms, introns and splice recognition sites. A cloned “gene” as used herein can be, e.g., a genomic or a cDNA gene, or a rRNA or tRNA gene, or the like.

After a gene of interest, or a portion thereof, has been cloned, defined mutation(s) can be introduced into it, using methods of site-specific mutagenesis which are well-known in the art. Any type of mutation, for example those defined above, can be introduced into a cloned gene of interest. In a preferred embodiment, a wild type, cloned M. marinum virulence gene is mutated such that an insertion or deletion (ranging from about 3 bases to about 90% of the entire gene sequence, preferably about 99 to about 4000 bases, most preferably about 500 bases) is introduced in such a way that the coding sequences remain in phase (i.e., the insertion or deletion is a multiple of 3 bases). In a most preferred embodiment, the mutation is an insertion of a nucleic acid fragment which comprises a kanamycin resistance marker. The site of the mutation can be chosen at will, but it is preferably in the 5′-terminal half of the gene. The availability of convenient restriction sites in the gene can simplify the introduction of mutations.

The mutated DNA can be reintroduced into the M. marinum genome by any of a variety of well-characterized methods. In a most preferred embodiment, the mutation is introduced into the genome by allelic exchange (homologous recombination). Methods for using long linear recombination substrates for allelic exchange in Mycobacteria are provided, e.g., in Balasubramanian, V. et al (1996). J. Bacteriol. 178, 273-279. Other methods for homologous recombination are found, e.g., in Aldovini, A. R. et al (1993). J. Bacteriol. 175, 7282-7289; Norman, E. et al (1995). Mol. Microbiol. 16, 755-760; Baulard, A. et al (1996). J. Bacteriol. 178, 3091-3098; Marklund, B. I. et al (1995). J. Bacteriol. 177, 6100-6105; Ramakrishnan, L. et al (1997). J. Bacteriol. 179, 5862-5868; and U.S. Pat. No. 5,700,683.

Simultaneously with the characterization of a virulence defect in an M. marinum mutant, or prior or subsequent to such characterization, the gene which is disrupted by the transposon insertion can be identified and characterized. In one embodiment, regions flanking one or both sides of an integrated transposon are characterized by hybridization to a panel of selected sequences. In a most preferred embodiment, the flanking regions are sequenced in order to identify the gene which has been disrupted. Many sequencing methods are, of course, well-known to those of ordinary skill in the art. Example 5 describes two methods to sequence directly the flanking regions, as well as methods to first clone and then sequence such regions. In a most preferred embodiment, genomic sequences flanking a transposon are amplified using a strategy called ligation-mediated PCR (LMPCR) (Prod'hom et al (1998). FEMS Microbiology Letters 158, 75-81). Briefly, this method uses one primer specific for the known sequence (IS (insertion sequence) present on both ends of the transposon) and a second specific for a synthetic linker ligated to restricted genomic DNA. This method is illustrated in FIGS. 11A and B. The size of the flanking regions which can be analyzed are limited by factors such as the fragment size that can be amplified by PCR, and can be readily determined by one of skill in the art. In a most preferred embodiment, a flanking region is about 100 to about 1,000 bases long.

The comparison of sequences of previously uncharacterized virulence genes in M. marinum to sequences in publicly available DNA and protein databases from a variety of sources (e.g., GenBank, EMBL, DDBJ, SWISS-PROT, PRF, PDB, RefSeq, etc.) can aid in the identification of (functional) homologues, and can add insight into the role a virulence gene plays in the molecular pathogenesis pathways of mycobacteria in an animal host.

Optimal alignment of sequences may be conducted by the local homology algorithm of Smith and Waterman (1981). Adv. Appl. Math. 2, 482; by the homology alignment algorithm of Needleman and Wunsch (1970). J. Mol. Biol. 48, 443; by the search for similarity method of Pearson and Lipman (1988). Proc. Natl. Acad. Sci. 85, 2444; or by computerized implementations of these algorithms (e.g., GAP, BESTFIT, FASTA, and TFASTA in the Wisconsin Genetics Software Package Release 7.0., Genetics Computer Group, 575 Science Dr. Madison, Wis.) Other such computer programs include, e.g., BLAST and FASTA (Altschul, S. F. et al (1990). J. Mol. Biol. 215, 403-410); BLASTX; TBLASTN; Gapped BLAST and PSI-BLAST (Altschul, S. F. et al (1997), Nucleic Acids Res. 25, 3389-3402). Alternatively, the sequences can be aligned by inspection. The best alignment (i.e., resulting in the highest percentage of sequence similarity over the comparison window) generated by the various methods is selected. In a most preferred embodiment, the BLAST blastx program is used.

Typically, a polynucleotide sequence of interest is translated into all six possible reading frames and is searched with the NCBI Blast search, selecting blastx. This translated sequence is first run against the EMBL data base to identify functional homologs. Then, if desired, the sequence is searched with the advanced Blast program, against Mycobacterium sequences in particular. In a preferred embodiment, sequences identified by such a homology alignment exhibit substantial identity to the sequence of interest. Of course, any selected degree of sequence identity can be the basis of such a comparison, e.g., about 30-50%, about 50-70% or about 70-90% sequence identity at the nucleotide or amino acid level.

The following terms are used to describe the sequence relationships between two or more polynucleotides or polypeptides: “reference sequence,” “comparison window,” “sequence identity,” “percentage of sequence identity,” and “substantial identity.”

A “reference sequence” is a defined sequence used as a basis for a sequence comparison; a reference sequence may be a subset of a larger sequence, for example, a segment of a full-length cDNA or gene sequence given in a sequence listing, or may comprise a complete cDNA or gene sequence. Generally, a reference is at least about 10 nucleotides in length, frequently at least about 20 to 25 nucleotides in length, and often at least about 50 nucleotides in length. In a preferred embodiment, a reference sequence is at least about 100 nucleotides in length, frequently at least about 150-300 nucleotides in length. Sequence comparisons between two (or more) polynucleotides are typically performed by comparing sequences of the two polynucleotides over a “comparison window” to identify and compare local regions of sequence similarity. A “comparison window,” as used herein, refers to a segment of at least about 10 contiguous nucleotide positions wherein a polynucleotide sequence may be compared to a reference sequence of at least about 10 contiguous nucleotides and wherein the portion of the polynucleotide sequence in the comparison window may comprise additions and deletions (i.e. gaps) of about 20 percent or less as compared to the reference sequence (which does not comprise additions or deletions) for optimal alignment of the two sequences.

The term “sequence identity” means that two polynucleotide or polypeptide sequences are identical (e.g., on a nucleotide-by-nucleotide or amino acid-by-amino acid basis) over the window of comparison. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the window of comparison (i.e., the window size), and multiplying the result by 100 to yield the percentage of sequence identity. The term “identical” in the context of two nucleic acid or polypeptide sequences refers to the residues in the two sequences which are the same when aligned for maximum correspondence.

The term “substantial identity” or “substantial similarity” indicates that a nucleic acid or polypeptide comprises a sequence that has at least about 90% sequence identity to a reference sequence, or preferably at least about 95%, or more preferably at least about 98% sequence identity to the reference sequence, over a comparison window of at least about 10 to about 100 or more nucleotides or amino acid residues. An indication that two polypeptide sequences are substantially identical is that one protein is immunologically reactive with antibodies raised against the second protein. An indication that two nucleic acid sequences are substantially identical is that the polypeptide which the first nucleic acids encodes is immunologically cross reactive with the polypeptide encoded by the second nucleic acid.

Another indication that two nucleic acid sequences are substantially identical is that the two molecules hybridize to each other under selected high stringent conditions. High stringent conditions are sequence-dependent and will be different with different environmental parameters. Generally, high stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, high stringent conditions will be those in which the salt concentration is at least about 0.2 molar at pH 7 and the temperature is at least about 60° C.

Analyses of the peptides or proteins which can be translated from flanking DNA sequences can be particularly informative for identifying functional homologues. The similarity between two polypeptides is determined by comparing the amino acid sequence and its conserved amino acid substitutes of one polypeptide to the sequence of a second polypeptide. Alignment procedures such as those discussed above can be used.

The sequencing and characterization of regions flanking a number of transposons which have independently integrated into M. marinum, rendering the bacteria avirulent in the goldfish model, is shown in Examples 9 and 10. At least some of the M. Marinum mutant genes are closely related to a previously identified functional homologue(s) from another organism, e.g., a transcriptional regulator from Streptomyces coelicolor which belongs to the AraC family of transcriptional regulators; polyketide synthase genes from Streptomyces and Pseudomonas bacteria; a sulfate adenylyltransferase with homology to diverse organisms including Pyrococcus abyssi, Synechocytis.sp and Bacillus subtilis; a cysQ gene, or dhbF from B. subtilis. The possible significance of these functional properties for M. marinum virulence is discussed in Example 9.

The flanking sequences in M. marinum are compared to databanks of mycobacteria sequences, using the Advanced Blast search from NCBI and selecting Mycobacterium as the genome, and/or the complete sequence of M. tuberculosis (Cole, S. T. et al (1998). Nature 393, 537-558), in order to identify virulence genes in other mycobacteria. In a most preferred embodiment, this method is used to identify virulence genes of M. tuberculosis.

For example, Examples 9 and 10 show that a number of the M. marinum virulence genes have functional homologues in M. tuberculosis, e.g. to members of the PPE/PE family; genes involved in fatty acid synthesis, aerobic metabolism, or amino acid synthesis; polyketide synthases; or regulatory genes. The possible significance of these functional properties for virulence is discussed in Examples 9 and 10.

Methods to clone such M. tuberculosis homologues are routine in the art. See, e.g., Example 7.

Defined mutations can be introduced into cloned, putative virulence genes of M. tuberculosis genes by methods similar to those discussed above for mutagenizing cloned M. marinum genes. The mutations can be made in M. tuberculosis either before or after the corresponding mutations in M. marinum have been characterized. Any of the types of mutations described above can be introduced into an M. tuberculosis gene, including knockouts of a large portion, including the entire coding sequence, of the gene. In order to facilitate the generation of mutants in M. tuberculosis, conventional, routine procedures can be used to identify those regions of the M. tuberculosis gene which correspond to the site of mutation in the corresponding M. marinum gene. For example, corresponding active sites and/or functional domains can be identified by, e.g., comparing the sequences or modeling the predicted protein structures. The mutated DNA can then be reintroduced into the M. tuberculosis genome by methods similar to those described above for reintroducing mutations into the M. marinum genome. Several such methods are described in Example 7. In a most preferred embodiment, the defined mutation is reintroduced into the M. tuberculosis genome by homologous recombination using a long linear recombination substrate. The phenotypic effect of an M. tuberculosis mutation can be determined routinely with one of several available animal models for this organism, including, e.g., the infection models with guinea pig (Collins, D. M. et al (1995). PNAS 92, 8036-8040; B. Bloom, ed., (1994). Tuberculosis: Pathogenesis, Protection, and Control, ASM Press, Washington, D.C. Chapter 9); mouse and rabbit (B. Bloom, ed., ibid, Chapters 8 and 10, respectively); and monkey (Walsh et al (1996). Nature Medicine 2, 430-436).

The invention encompasses virulence genes (e.g., isolated virulence genes) as described elsewhere herein, from M. marinum and/or M. tuberculosis, which are identified by the methods of the invention, and/or variants (e.g., naturally- or non-naturally-occurring modifications, mutations, polymorphisms, etc.) or fragments thereof. By a “variant” of a gene or fragment is meant, as used herein, a replacement, deletion, insertion or other modification of the gene or fragment. It is preferred that the variant has at least about 70% sequence identity, more preferably at least about 85% sequence identity, most preferably at least about 95% or 98% sequence identity with the gene or fragment. The degree of similarity can be determined using any of the methods disclosed herein. By a “fragment” of a gene is meant a single strand or double stranded nucleic acid (e.g., oligonucleotide) of a size smaller than that of the gene, obtained by any of a variety of conventional means, e.g., digestion with restriction enzymes, PCR amplification, synthesis with an oligonucleotide synthesizer, synthesis with a DNA or RNA polymerase, or the like. Such fragments can be used, for example, to diagnose the presence of a gene in a sample of interest, e.g., by serving as a hybridization probe or a PCR primer. Such diagnostic assays can be set up and performed by routine, conventional procedures in the art. In another embodiment, such fragments can be used to screen for virulent strains of bacteria, e.g., bacteria which comprise a polynucleotide that encodes a particular virulence gene or a fragment thereof. Of course, full-length virulence genes of the invention and variants thereof can also be used in diagnostic assays.

The invention also encompasses polynucleotides which are complementary to a gene of the invention or fragment thereof, or which hybridize to such a gene or fragment under selected (e.g., high) stringency conditions. For example, the invention encompasses an oligonucleotide complementary to a portion of a virulence gene which can be used, e.g., as an antisense oligonucleotide to regulate expression of the gene, e.g., in a method of therapy. Methods to make and use antisense molecules of this type are conventional and routine, and are presented, e.g., in U.S. Pat. Nos. 5,876,931 and 5,585,479 and in references cited therein. Similarly, ribozymes comprising such fragments can be used in a method of treatment. Methods of making and using ribozymes are also conventional in the art.

Of course, the genes and fragments discussed herein can be any form of polynucleotide or nucleic acid, e.g., naturally occurring, synthetic or intentionally manipulated polynucleotides, wherein nucleotide bases or modified bases are linked by various known linkages, e.g., ester, phosphodiester, sulfamate, sulfamide, phosphorothionate, phosphoroamidate, methyl phosphonate, carbamate, or other bonds, depending on the desired purpose, e.g., resistance to nucleases, such as RNAse H, improved in vivo stability, etc. Various modifications can be made to nucleic acids, such as attaching detectable markers (e.g., avidin, biotin, radioactive or fluorescent elements, ligands), or moieties which improve hybridization, detection or stability. The polynucleotides can be DNA, cDNA, RNA, PNA, synthetic nucleic acid, modified nucleic acid, or mixtures thereof. Polynucleotides can be of any size, e.g., ranging from short oligonucleotides to large gene clusters or operons. Either or both strands of a double strand nucleic acid are included.

The invention also encompasses peptides or polypeptides encoded by and/or expressed from M. marinum and/or M. tuberculosis genes identified by the methods of the invention, and/or variants or fragments thereof, and products which are generated by such peptides or polypeptides. The term “genes identified by the methods of the invention” encompasses any gene in a given operon, a mutation in one of whose genes results in an avirulent phenotype (e.g., the gene can be a downstream gene whose expression is diminished or abolished because of an upstream polar mutation, or a gene whose gene product interacts with another gene product of the operon, etc.).

The peptides or polypeptides can be isolated (e.g., purified) from bacteria directly, or they can be expressed recombinantly and isolated (e.g., purified) from recombinant organisms. Methods of isolating, purifying and sequencing naturally produced or recombinantly produced peptides and polypeptides are conventional and routine in the art. The genes can be cloned into any of a variety of expression vectors. The sequences to be expressed can be genomic sequences, e.g., subcloned sequences from a cosmid library as described in Example 6, or they can be corresponding cDNA sequences, obtained by conventional means. In some cases, it may be desirable to express a fragment of a gene, or more than one gene, e.g., as many as the genes of an entire operon. Vectors and appropriate regulatory elements for expressing genes in a variety of cell types or hosts, including prokaryotes, yeast, and mammalian, insect and plant cells, and methods of cloning and expressing genes or gene fragments, are routine in the art and are discussed, e.g., in U.S. Pat. Nos. 5,876,931, 5,700,683, 4,440,859, 4,530,901, 4,582,800, 4,677,063, 4,678,751, 4,704,362, 4,710,463, 4,757,006, 4,766,075 and 4,810,648.

Virulence polypeptides (or fragments thereof) expressed recombinantly may be used in a variety of assays. For example, such a polypeptide can be used as a reagent in an immunoblot assay to test whether a patient (subject) has been exposed to a virulent M. marinum or M. tuberculosis (i.e., to test whether the patient has antibodies to the detection polypeptide). The virulence polypeptides of the invention can also be used as antigenic vaccine components to direct antibodies to elements which are important for virulence. The polypeptides can be added to existing vaccines (e.g., avirulent bacteria as discussed elsewhere herein) to supplement the range of antigenicity conferred by the vaccine, or may be used apart from other mycobacterial antigens.

The invention also encompasses a host transformed to express a peptide or polypeptide of the invention, or a host which is mutated so the expression of a peptide or polypeptide of the invention is disrupted (e.g., inhibited), or progeny of such hosts.

“Variants” of the peptides or polypeptides are also included in the invention, e.g., insertions, deletions and substitutions, either conservative or non-conservative, where such changes do not substantially alter the normal function of the protein. By “conservative substitutions” is meant by combinations such as Gly, Ala; Val, Ile, Leu; Asp, Glu; Asn, Gln; Ser, Thr; Lys, Arg; and Phe, Tyr. Variants can include, e.g., homologs, muteins and mimetics. Many types of protein modifications, including post-translational modifications, are included. See, e.g., modifications disclosed in U.S. Pat. No. 5,935,835.

“Fragments” of the peptides or polypeptides are also included in the invention. These fragments can be of any length. In a preferred embodiment, a fragment is functional (e.g., has biological activity, can inhibit or enhance the activity of a protein or other substance, contains one or more immunogenic epitopes, etc.). In a most preferred embodiment, the fragment contains all or a subset of the amino acids of SEQ ID NOs: 5, 24, 26, 52, 54, 56, 58, 60, 62, 64, 66, 68, 70, 72, 74, 76, 78, 80, 82, 884, 86, 88, 90, 92, 94, 96, 98, 100, 102, 104, 106, 108, 110, 112, 114, 116 or 118.

Among the polypeptides of particular interest are polyketide synthases. Examples 10 and 11, for example, shows that M. tuberculosis homologues of the invention are polyketide synthase genes. As is well-known, many polyketides have therapeutic value (for human, veterinary, or aquaculture uses). For example, polyketides have been shown to function as antibiotics, chemotherapeutic agents or immunosuppressive agents, e.g., in transplant patients. The invention includes the generation and/or isolation (e.g., purification) of polyketide synthases encoded by virulence genes identified by the method of the invention, as well as polyketides produced by those synthases. The polyketides can be generated by recombinant means, isolated from non-recombinant bacteria, or produced synthetically. Methods for making, isolating and purifying polyketides are routine and well-known in the art.

Recombinantly expressed polypeptides of the invention can also be used to confirm that a particular virulence gene is responsible, at least in part, for a pathogenic phenotype in an organism—that is, to confirm Koch's postulates. Example 8 shows how a recombinantly expressed M. marinum putative virulence gene can be used to complement a mutant bacterium which is defective in that gene, and to restore a virulent phenotype in fish infected by the complemented mutant.

Virulence genes of the invention and peptides thereof can contain antigenic epitopes. The invention also encompasses antibodies, including polyclonal or monoclonal antibodies, or fragments of polyclonal or monoclonal antibodies, which are generated in response to such epitopes. Such antibodies can be used, e.g., in diagnostic assays to detect the presence of a mycobacterium, to identify virulent strains of bacteria, or in methods to treat disease conditions caused or exacerbated by a virulence protein (e.g., passive immunization), following routine, art-recognized procedures.

The invention also encompasses an avirulent mycobacterium, preferably M. marinum and/or M. tuberculosis, which harbors one or more mutation(s) in one or more virulence gene(s) identified by the methods of the invention, or a pharmaceutical composition which comprises such a bacterium and a pharmaceutically acceptable carrier.

In a preferred embodiment, the avirulent bacterium is introduced into a host (e.g., a fish, cow or human) in order to elicit an immune response. Because the bacterium is avirulent (e.g., attenuated), it is expected to be suitable for administration to a host in need of treatment, but it is also expected to be antigenic and to give rise to an immune response, preferably a protective immune response. For such a use, it is preferred that the mutation is substantially non-revertable, e.g., a deletion or frame-shift mutation. To ensure non-revertability, it is preferable that a bacterium comprises at least two or three such mutations, preferably in different genes. A small deletion mutant would be expected to provide antigenic epitopes in the portion of the protein which lies downstream of the deletion, even though the protein, itself, is not functional with respect to virulence.

Another embodiment of the invention is a vaccine comprising a suitable avirulent mycobacterium of the invention and a pharmaceutically acceptable carrier. By vaccine is meant an agent used to stimulate the immune system of a living-organism so that protection against future harm is provided. Immunization refers to the process of inducing an antibody and/or cellular immune response in which T-lymphocytes can either kill the pathogen and/or activate other cells (e.g., phagocytes) to do so in an organism, which is directed against a pathogen or antigen to which the organism has been previously exposed. The term “immune response,” as used herein, encompasses, for example, mechanisms by which a multi-cellular organism produces antibodies against an antigenic material which invades the cells of the organism or the extra-cellular fluid of the organism. The antibody so produced may belong to any of the immunological classes, such as immunoglobulins A,D,E,G or M. Other types of responses, for example cellular and humoral immunity, are also included. Immune response to antigens is well studied and widely reported. A survey of immunology is given e.g., in Roitt I., (1994). Essential Immunology, Blackwell Scientific Publications, London. Methods in immunology are routine and conventional (see, e.g., in Current Protocols in Immunology; Edited by John E. Coligan et al., John Wiley & Sons, Inc.).

Methods of formulating, testing, optimizing and administering vaccines of the invention are routine and conventional, and are described, e.g., in U.S. Pat. Nos. 5,876,931, 5,700,683, and references cited therein, and in “New Generation Vaccines, edited by M. M. Levine et al, 2nd edition, Marcel Dekker, Inc., New York, N.Y., 1997.” Active immunization of a patient (e.g., human, fish, cow, etc.) is preferred. In this approach, one or more mutant bacteria are prepared in an immunogenic formulation containing suitable adjuvants and carriers and administered to the patient in known ways. Suitable adjuvants include Freund's complete or incomplete adjuvant, muramyl dipeptide, the “Iscoms” of EP 109 942, EP 180 564 and EP 231 039, aluminum hydroxide, saponin, -DEAE-dextran, neutral oils (such as miglyol), vegetable oils (such as arachis oil), liposomes, Pluronic polyols or the Ribi adjuvant system (see, for example GB-A-2 189 141). “Pluronic” is a Registered Trade Mark. The patient to be immunized is a patient requiring to be protected from the disease caused by, or exacerbated by, the virulent form of the bacterium.

The aforementioned avirulent bacteria of the invention or a formulation thereof may be administered by any conventional method including oral and parenteral (e.g., subcutaneous or intramuscular) injection. The treatment may consist of a single dose or a plurality of doses over a period of time. While it is possible for an avirulent bacterium of the invention to be administered alone, it is preferable to present it as a pharmaceutical formulation, together with one or more acceptable carriers. The carrier(s) must be “acceptable” in the sense of being compatible with the avirulent microorganism of the invention and not deleterious to the recipients thereof. Typically, the carriers will be water or saline which will be sterile and pyrogen free.

It will be appreciated that a vaccine of the invention, depending on its bacterial component, may be useful in the fields of human medicine, veterinary medicine, or aquaculture. A vaccine for fish against Mycobacterium marinum could be of particularly significant economic importance. Mycobacterium marinum causes tuberculosis in more than 150 species of both salt-water and fresh-water fish, among them salmonid trout (salmo gairdneri, salmo trutta, oncorhynchos mykiss), striped bass, tilapia, etc. Aquaculture facilities infected with M. marinum suffer from a constant mortality rate over a long period of time accompanied by severe economic losses, which could be ameliorated with such a vaccine. A vaccine against M. tuberculosis could, of course, be a significant weapon in the battle against tuberculosis, which is wide-spread in human populations.

Vaccines encompassed by the invention also include killed bacterial vaccines; subunit vaccines comprising a virulence protein(s) of the invention (e.g., a wild type or mutant protein(s), or a variant(s) thereof), or an antigenic fragment(s) thereof; bacteria or viruses which produce or are capable of producing such virulence proteins or fragments; and DNA vaccines comprising a nucleic acid which encodes such a virulence protein or fragment thereof. Methods of making and using such vaccines are routine and conventional in the art. For methods of making and using DNA vaccines, see, e.g., U.S. Pat. No. 5,589,466.

An avirulent bacterium of the invention can also be used as a “carrier” for the expression of one or more cloned heterologous gene(s) or fragments thereof. For example, an avirulent M. marinum organism can be used to express a secreted or surface-expressed heterologous peptide or polypeptide in fish, and an avirulent M. tuberculosis organism can be so used in humans. The avirulent bacterium can be used to express, e.g., an allergen, or an antigenic epitope from another pathogen, for which the modified bacterium can act as a vaccine, or to express a gene for use in a method of gene therapy. Appropriate therapeutic genes and methods of using such bacteria in methods of gene therapy are well-known in the art. In a preferred embodiment, the heterologous gene is inserted at or near the position at which the transposon was inserted in an avirulent mutant, or at or near the site of the more “well-defined” avirulent mutation. The heterologous gene may be placed under the control of an endogenous or heterologous, constitutive or inducible, promoter, many of which are well-known in the art. Methods to clone heterologous genes are routine, as are methods to express them in a host. Methods of making and using such carriers are disclosed, e.g., in U.S. Pat. Nos. 5,876,931 and 5,424,065.

The invention also encompasses a method for identifying an agent which reduces the ability of a microorganism to survive in a host, e.g., an anti-mycobacterial agent which inhibits expression of a virulence gene, or which attacks products produced directly or indirectly by a virulence gene. Such an agent can, e.g., inhibit or interfere with transcription, translation, or post-translational processing of a virulence gene (either from M. marinum or M. tuberculosis) of the invention, or can inhibit or interfere with the activity of a virulence protein of the invention. In a preferred embodiment, such an agent can be used to treat a disease caused by, or exacerbated by, a virulence gene of the invention, or can serve as a prophylactic agent to inhibit mycobacterial virulence.

One such method, as disclosed, e.g., in U.S. Pat. No. 5,876,931, is to generate a bacterium which over-expresses the virulence gene, and then to identify an agent which reduces the viability or growth of a wild type cell but not the cell overexpressing the gene, in a host. Methods to generate the over-expressing strain, and to perform such screening procedures, are routine and are described, e.g., in U.S. Pat. No. 5,876,931.

Other methods to screen for anti-mycobacterial drugs are routine and are described, e.g., in U.S. Pat. No. 5,700,683. For example, the use of reporter genes such as, e.g., firefly luciferase, beta galactosidase, GFP or the like, placed under the control of a regulatory sequence (e.g., a promoter) of a virulence gene of the invention, provides a rapid assay for an agent that regulates said promoter. In other assays, a reporter gene can be fused in phase to a portion of a virulence gene of the invention.

Anti-mycobacterial agents characterized by such methods can be of any of a variety of forms, e.g., small molecules, or antibodies generated against virulence proteins of the invention or antigenic fragments thereof. Other anti-mycobacterial agents include, e.g., antisense oligonucleotides, ribozymes specific for one of the virulence genes of the invention, decoy genes, transdominant proteins and suicide genes (for all of which methods of making and using are conventional and well-known in the art).

A ribozyme is a catalytic RNA molecule that cleaves other RNA molecules having particular nucleic acid sequences. Ribozymes useful in this invention are those that cleave, e.g., transcripts encoding virulence proteins of the invention. Examples include hairpin and hammerhead ribozymes.

A decoy nucleic acid is a nucliec acid having a sequence recognized by a regulatory DNA binding protein (i.e., a transcription factor). Upon expression, the transcription factor binds to the decoy nucleic acid, rather than to its natural target in the genome. Useful decoy nucleic acid sequences include any sequence to which a transcription factor binds in a virulence gene of the invention.

A transdominant protein is a protein whose phenotype, when supplied by transcomplementation, will overcome the effect of the native form of the protein. For example, an avirulent mycobacterium can be rendered virulent by introducing a transdominant protein such as one of the genes of the invention, or a virulent mycobacterium can be rendered avirulent by introducing a virulence gene of the invention that has been inactivated, e.g., by insertional mutagenesis.

A suicide gene produces a product which is cytotoxic. In the vectors of the presnt invention, a suicide gene is operably linked to an inducible expression contol sequence which is stimulated upon infection of a cell by an M. marinum or M. tuberculosis.

The invention also relates to a method of screening vaccine candidates for human tuberculosis in the fish model. In one embodiment, based on the assumption that M. marinum bacteria may be suitable for human vaccines, goldfish can be inoculated with an M. marinum vaccine candidate of interest. The fish are then challenged with fully virulent M. marinum at a dose capable of establishing disease. A vaccine which, when inoculated into a fish, protects the fish from subsequent virulent challenge by the fish failing to develop disease symptoms is a candidate for a human vaccine. In another embodiment, a putative virulence gene of M. tuberculosis is selected, and a mutation is made in the M. marinum homologue of that gene. The mutant M. marinum is then tested as a vaccine candidate, using the goldfish model as above.

The invention also relates to a method of detecting the presence of an M. marinum or M. tuberculosis infection in a subject, comprising administering to the subject (e.g., by intracutaneous administration or by multiple puncture technique) one or more virulence proteins of the invention, or an antigenic fragment thereof, and determining if, e.g., cell-mediated immunity occurs. That is, a “skin test” is performed. Such a test can allow, e.g., for the early detection of a patient at risk for tuberculosis.

In another embodiment, the presence of an M. marinum or M. tuberculosis infection in a subject can be detected by means of specific oligonucleotide probes against one or more virulence genes of the invention, using conventional hybridization assays.

In another embodiment, the presence of an M. marinum or M. tuberculosis infection in a subject can be detected by screening for the presence in the blood of antibodies against one or more of the virulence proteins of the invention. Conventional immunological assays can be performed.

### BRIEF DESCRIPTION OF THE FIGURES

FIG. 1 shows the median survival time (MST) of fish inoculated with M. marinum. The median survival time of fish (days) inoculated with M. marinum at doses indicated per fish is compared to a phosphate buffered saline (PBS) control. *survival to endpoint of experiment, 56 days.

FIG. 2 shows a comparison of the growth of M. marinum in liver, spleen and kidney. The inoculum is 10⁷ CFU/fish. Results are given as geometric means±standard error for eight fish per time point.

FIG. 3 shows a comparison of mean cumulative granuloma scores (MCGs) over time of fish infected with 10⁷ CFU of M. marinum organisms. The results are given as a vertical box plot, with horizontal lines marking the median 10^{th}, 25^{th}, 50^{th}, 75^{th} and 95^{th} percentile points of GSs for eight animals at each time point. The mean of each group is represented by a thick line. At 2 weeks, the median 50^{th} percentile and mean values are the same.

FIG. 4 shows a survival curve of goldfish inoculated with 10⁸ CFU of M. marinum 1218R (wild type) or 1218S (mutant).

FIG. 5 shows the modification of pYUB285 with transposon tags. Bg is BglII; Bam is BamH1; H is HindIII; IR are inverted repeats which mark the boundaries of the transposon; ORFR and ORFA are transposon genes; aph is the gene for kanomycin resistance; oriE is the E. coli ori; and ΔoriM is the disabled mycobacterial ori.

FIG. 6 shows the construction of an M. marinum signature-tagged mutant library.

FIG. 7 shows a schematic diagram of an M. marinum mutant library screen in the goldfish model.

FIG. 8 shows a survival curve of M. marinum mutant 41.2.

FIG. 9 shows a survival curve of M. marinum mutant 80.1.

FIG. 10 shows a survival curve of M. marinum mutant 86.1.

FIGS. 11A and B illustrate ligation-mediated PCR.

FIG. 12 shows Competitive Indices of M. marinum mutants 32.2, 60.2, 62.2, 67.1, 80.1, 86.1, 42.2, 80.8 and 68.6.

FIG. 13 shows a survival curve of M. marinum mutant 67.1.

FIG. 14 shows a survival curve of M. marinum mutant 39.2.

FIG. 15 shows a survival curve of M. marinum mutant 42.2.

FIG. 16 shows competitive index data for some mutants.

### EXAMPLES

### Example 1

### Properties of the M. marinum/Goldfish Model

A. Median Survival Time and LD₅₀.

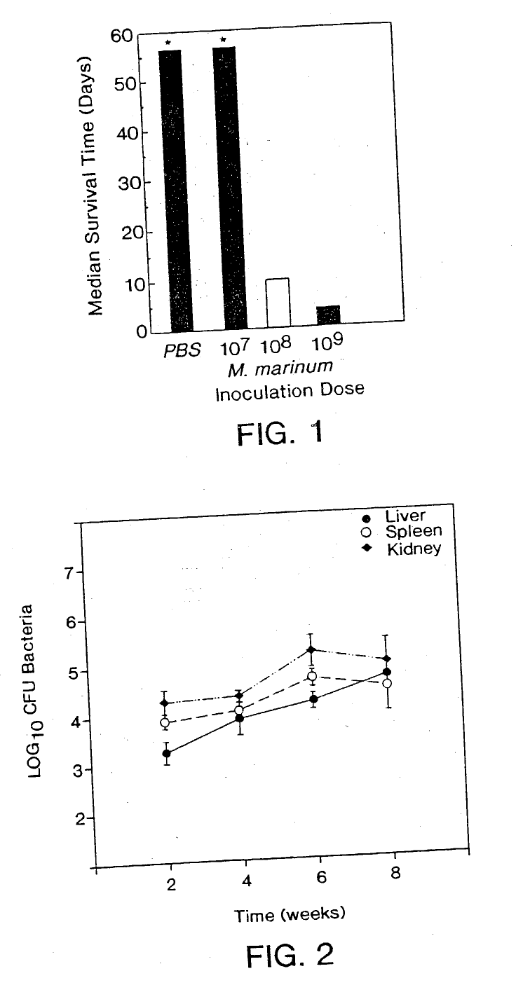

To determine the median survival time of goldfish after inoculation with M. marinum strain ATCC 927, groups of 20 to 32 fish were inoculated intraperitoneally with 10⁹, 10⁸, or 10⁷ colony forming units (CFU). The median survival time of goldfish inoculated with M. marinum was dose dependent, with survival time decreasing with increasing doses of bacteria. The median survival time of fish was 4, 10, and >56 days (the endpoint of the experiment) with inocula of 10⁹, 10⁸, or 10⁷ M. marinum organisms, respectively. All fish inoculated with 10⁷ CFU or less survived to the end point of the experiment (56 days). The control fish group, inoculated with PBS in 5 separate experiments, had a total of two premature deaths, one at 8 and one at 19 days post-inoculation, from a total of 55 fish. The remainder of the control fish survived to 56 days, the endpoint of the experiment (See FIG. 1). The LD₅₀ at 1 week postinfection with M. marinum was 4.5×10⁸ (calculated by the method of Reed & Muench, 1938. Am. J Hyg. 27, 493-497).

B. Mycobacterial Recovery from Fish Organs.

To assess the ability of M. marinum to persist in goldfish tissue, the liver, spleen, and kidneys from each sacrificed fish were collected for bacteriological examination. M, marinum was recovered from all organs of fish in the 10⁹ or 10⁸ CFU inoculum groups. In fish inoculated with 10⁷ CFU, M. marinum was recovered from 96% of the examined organs.

The fate over an 8 week period of the M. marinum ATCC 927 strain in the livers, spleens, and kidneys of fish inoculated with 10⁷ CFU was followed. (See FIG. 2). There was a significant positive linear relationship between time postinoculation and colony recovery in the liver (P<0.001); for the spleen and kidneys, the relationship was positive but did not reach statistical significance (P=0.054 and P=0.091, respectively). Between 8 and 16 weeks postinoculation, M. marinum persisted in the tissue with no significant change in the colony counts. In addition, in the 10² to 10⁶ CFU inoculum groups, M. marinum was isolated from at least one organ from all infected fish.

C. An Acute and Chronic Form of Mycobacterial Infection.

The pathology of infected fish was dependent on the inoculum dose and the time postinfection of animal sacrifice. Fish infected with either 10⁹ or 10⁸ CFU of M. marinum organisms suffered from anorexia, sluggish movement, and loss of equilibrium.

The histopathology of fish infected with 10⁹ and 10⁸ CFU was characterized by severe peritonitis and necrosis as compared to control fish. The peritoneum was filled with inflammatory cells consisting of lymphocytes, macrophages, fibrous connective cells as well as with degenerating cells and bacteria. The mean cumulative granuloma score (MCGS) for these 2 groups was similar (0.2 for the 10⁹ CFU group and 0.9 for the 10⁸ CFU group). In the 10⁸ CFU inoculum group, granuloma formation was more likely to be found in animals which survived more than 2 weeks postinoculation.

When examined at 2 weeks, 6 of 8 fish in the 10⁷ CFU group had moderate to severe peritonitis. Unlike the 10⁸ and 10⁹ CFU inoculum groups which succumbed to infection, the 10⁷ CFU inoculum group survived the infection, and by 4 to 6 weeks postinoculation, the acute peritoneal inflammation was replaced by a chronic inflammatory state. Fish inoculated with 10⁷ CFU demonstrated granuloma formation in all organs evaluated (MCGS of 5.0), including the peritoneum and pancreas, liver (e.g., onion ring granuloma composed of epithelioid macrophages surrounding a necrotic center), spleen, trunk kidney, head kidney, heart and intestine. Pleomorphic granulomas (necrotizing, non-necrotizing and caseous) were seen. The necrotizing granulomas were characterized by a central area of necrosis surrounded by macrophages, epithelioid cells, and thin fibrous connective tissue. Frequently, caseous necrosis was present in the central area of the granuloma. Granulomas containing foamy macrophages were also seen. Occasionally, Langhans and foreign body type giant cells were observed. In addition, acid fast bacilli could be demonstrated with the modified Ziehl-Neelsen stain. Melanomacrophage centers were seen in a few cases.

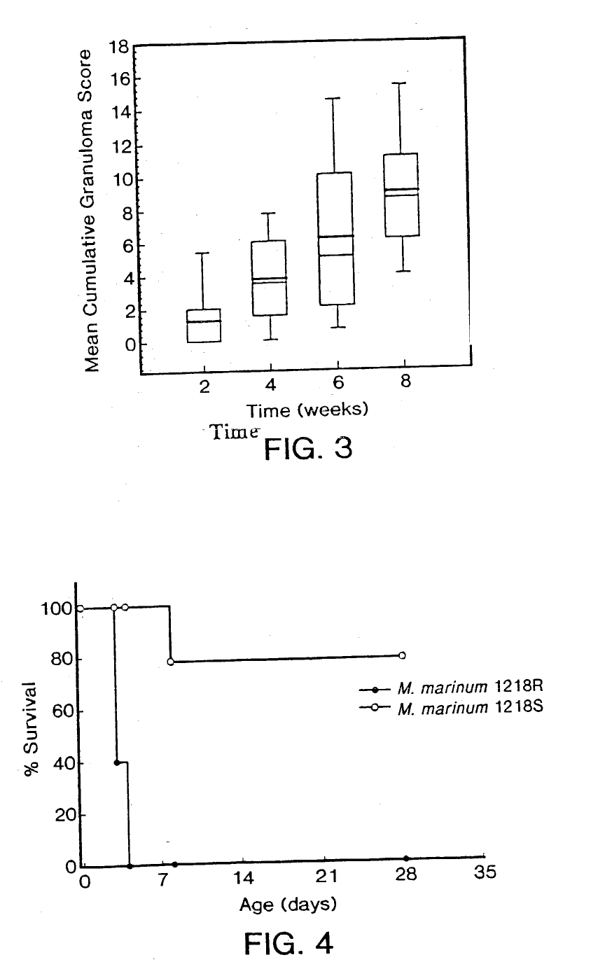

The chronic inflammatory response of fish towards M. marinum was time dependent, as seen by the increment in mean cumulative granuloma scores (MCGSs) with time in animals inoculated with 10⁷ CFU (See FIG. 3) up to 8 weeks. From 8 to 16 weeks postinoculation, there was no significant change in MCGSs (5.0 and 5.7 respectively).

D. Minimum Infectious Dose (MID).

To estimate the lowest possible dose of M. marinum able to establish infection in goldfish, groups of four fish were inoculated with M. marinum ATCC 927 at doses of 10⁶, 10⁵, 10⁴, and 10² CFU. Granuloma formation was seen in 25% of the goldfish by 4 weeks and in 88% by 8 weeks postinfection with a dose of 6.3×10² CFU or higher (Table 1). The minimum number of organisms required to establish infection in goldfish appears to be approximately 600 CFU.

TABLE 1
MID of M. marinum ATCC 927

No. positive^{a}

| Inoculum (CFU/fish) | 4 Wk | 8 Wk | MCGS |
|---|---|---|---|
| 1.2 × 10⁶ | 1/2 | 1/2 | 5.0 |
| 3.0 × 10⁵ | 0/2 | 2/2 | 5.5 |
| 2.4 × 10⁴ | 1/2 | 2/2 | 1.5 |
| 6.3 × 10² | 0/2 | 2/2 | 4.5 |

^{a}Number of granuloma-positive animals per total number of animals at 4 and 8 weeks postinoculation.

Mycobacterial Virulence Assay.

The relative virulence of different strains of M. marinum, isolated from both human and animal origin, was assessed. Three mycobacterial strains, M. marinum ATCC 927, M and F-110, were inoculated into goldfish at 10⁸ CFU. The median survival times of M, marinum M, ATCC 927, and F-100 were similar, ranging from 4 to 10 days.

### Example 2

### Differentiation of an Avirulent M. marinum Mutant from the Wild Type in the Goldfish Model

The goldfish model can differentiate between virulent and avirulent M. marinum organisms. A comparison of such a pair of strains is shown in FIG. 4. The M. marinum strains designated 1218R (wild type, aka ATCC 927) and 1218S (avirulent mutant) were inoculated into groups of 5 to 9 goldfish in two separate experiments at an inoculum dose of 1.4 to 4×10⁸ CFU. The median survival time of goldfish inoculated with M. marinum 1218R organisms was 3 days compared to 28 days (endpoint of experiment) with M. marinum 1218S organisms (See FIG. 4). The mutant 1218S also failed to persist in the mouse macrophage model. This experiment shows that the fish mycobacteriosis model can allow the identification of M. marinum virulence genes.

### Example 3

### Signature-Tagged Mutagenesis, and the Generation of a Library of Mutants

A. Construction of a Master Bank of Signature-Tagged Transposons

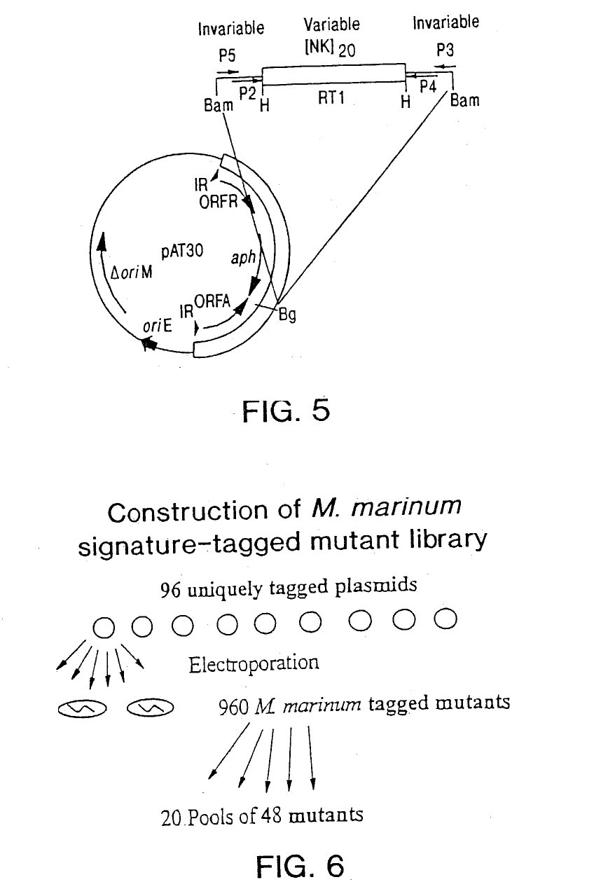

As an initial step in creating a bank of signature-tagged transposons, plasmid pAT30 is generated (see FIG. 5). A unique restriction site (BglII) is introduced into the mycobacterial transposon delivery vector pYUB285 between ORFA and aph. The vector is a suicide vector in mycobacteria because of inactivation of the mycobacterial origin of replication by an internal deletion. A kanamycin resistance gene (aph) inserted into IS1096 allows for a library of insertions in the mycobacterial genome to be generated upon electroporation of the plasmid followed by selection for kanamycin.

To generate a collection of signature tagged transposons to be inserted into pAT30, primers P5 (5′-CTAGGTACCTACAACCTC-3′) (SEQ ID NO: 1) and P3 (5′-CATGGTACCCATTCTAAC-3′) (SEQ ID NO: 2) and the template RT1 oligonucleotide (5′-CTAGGTACCTACAACCTCAAGCTT-[NK]₂₀ AAGCTTGGTTAGAATGGGTACCATG-3′) (SEQ ID NO: 3) are prepared by conventional, routine methods, preferably using a commercially available oligonucleotide synthesizer. The 5′ ends of primers P5 and P3 have BamHI sites. The template RT1 oligonucleotide is similar to that designed by Hensel et al, with a variable central region (NK)₂₀ flanked by arms of invariant sequences. The invariant arms allow the sequence tags to be amplified in a PCR with the use of primers P3 and P5. The variable region is designed to ensure that the same sequence occurs only about once in 2×10¹⁷ molecules. PCR is performed, using standard, routine methods (see, e.g., Innis, M. A. et al., eds. PCR Protocols: a guide to methods and applications, 1990, Academic Press, San Diego, Calif.) to generate and amplify double stranded, 90 bp signature tags. The PCR amplified tags are digested with BamHI, gel purified, and then ligated to the BglII digested, dephosphorylated (calf intestinal phosphatase, New England BioLabs, Inc.) pAT30 plasmid. E. coli DH5a is transformed with this ligation mixture and plasmids from 800 individual clones are isolated, arrayed in 96 well microtiter plates, and transferred to nylon membranes. These plasmids are analyzed for hybridization and tag amplification efficiency. In this example, ninety-six plasmids that are hybridization and amplification efficient are chosen for the master plasmid collection. The master plasmids are screened for cross hybridization with other plasmids in the master plasmid collection and any cross-hybridizing plasmids are eliminated until the collection has no cross hybridizing members. Of course, a master plasmid collection of any size can be constructed by this method. Methods for carrying out STM mutagenesis and isolating bacterial virulence mutants are described, e.g., in Hensel et al (1995). Science 269, 400-403 and U.S. Pat. No. 5,876,931.

B. Optimization and Initial Characterization of M. marinum Transposition

Several protocols for the preparation of competent cells from M. marinum are evaluated. The strains tested are ATCC 927 (fish isolate) and M. marinum strain M (human isolate). Electrocompetent cells are prepared from M. marinum cells grown to different growth phases at different temperatures in the presence or absence of ethionamide or cycloheximide. Mycobacterial cells are transformed by electroporation with the replicative Escherichia coli—mycobacteria shuttle vector, pYUB18 (Jacobs, W. R. et al (1991). Methods Enzymol 204, 537-555), as well as the suicide vectors pYUB285 (McAdam R. A. et al (1995). Infect. Immun. 63, 1004-1012) and pUS252, carrying the transposable elements, IS1096 and IS6110, respectively (Dale, J. W. (1995). Eur. Respir. J. 8, 633s-648s). Mutants of M. marinum are recovered on 7H10 agar plates supplemented with kanamycin. Transformation and transposition efficiencies under different protocols are compared, using routine, art-recognized procedures. See, e.g., McAdam et al (1995). Infec. Immun. 63, 1004-1012 and Cirillo, J. D. et al (1991). J. Bacteriol. 173, 7772-7780. Southern hybridization analysis is performed on mycobacterial mutants to confirm the transposition events. These analyses show that: 1) competent cells prepared at room temperature from late-exponential growth phase organisms yield a higher transposition efficiency than cells prepared at 4° C. or from early-or mid-exponential growth phase organisms; 2) the highest efficiency for transposition is 10²-10³ cfu per μg of plasmid DNA; and 3) the IS1096-derived transposon is best able to efficiently mutagenize M. marinum.

To confirm that M. marinum-kanamycin resistant colonies are not spontaneous mutants, colonies recovered after electroporation with the non-integrating, replicative vector, pYUB18, are analyzed; the plasmid pYUB18 is successfully isolated from 6 separate transformants and is identified by restriction enzyme mapping. This indicates that the transformants are not spontaneous mutants. In another experiment, 35 randomly selected mutants recovered from electroporation of the suicide vector, pYUB285 are examined by Southern analysis to determine whether transposition is random in the M. marinum chromosome. All tested transposon mutants yield a single band, located in a different position on the Southern blot, consistent with random integration of a single copy of IS1096 into the M. marinum genome. Evaluation of 10 mutants obtained in a single electroporation experiment shows that each mutant is inserted into a different part of the M. marinum genome, indicating that the mutants from a given electroporation do not represent siblings.

C. Generation of an M. marinum Mutant Library

An M. marinum mutant library is generated by electroporating individual members of the 96 master plasmid collection into M. marinum bacteria (See FIG. 6). M. marinum electrocompetent cells are prepared from a 100 ml culture grown to late exponential phase (O.D.₆₀₀=1.6 to 1.8). Bacteria are washed three times at room temperature with 10% glycerol and then suspended in 1 ml 10% glycerol and distributed to 0.2 cm gap electroporation cuvettes (Bio-Rad Laboratories). Electroporation is performed at room temperature using a Gene Pulser (Bio-Rad Laboratories) with parameters of 2.5 kV, 25 μF, and 800 Ω. Electroporated cells are rescued by growth overnight in 7H9 broth with 10% albumin-dextrose complex enrichment (ADC) (52) at 30° C. and plated on 7H10 agar with kanamycin (20 μg/ml) and incubated at 30° C. Mutants appear 1 to 2 weeks after plating. Mutants from each electroporation are named for the master plasmid used for transposon delivery (pAT30-1 plasmid yields mutants 1.1, 1.2, etc.). In this example, 960 mutants are isolated, 10 mutants per master plasmid. Of course, more mutants can be isolated per each master plasmid, and the 96 (or additional) master plasmids can be used to generate additional mutants.

### Example 4

### Screening an M. marinum Library for Potential Avirulent Mutants, Using the Goldfish Model

A. Screening for Mutants which Show Reduced Viability in the Goldfish Host

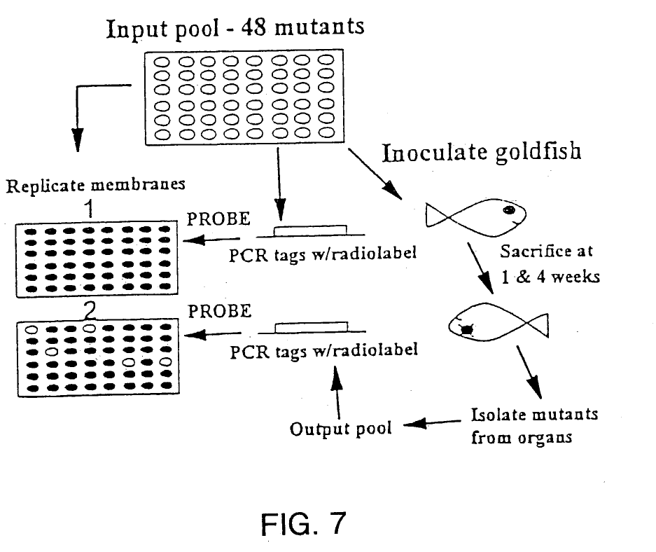

The M. marinum library obtained in Example 3 is screened for mutants which exhibit a reduced ability to survive in the goldfish model. The library of M. marinum transposon-tagged mutants is screened in pools; in this example, each pool has 48 mutants (See FIG. 7). Each of the mutants in a given pool is marked with a unique DNA tag (i.e. they are derived from 48 of the 96 master plasmids). To generate an input pool, mutants that make up the pool are grown in individual wells of a 96-well microtiter plate containing 7H9 broth with ADC and kanamycin (20 μg/ml) at 30° C. until they reach O.D.₆₀₀=0.6-0.8. The mutants are then pooled and an aliquot is removed for amplification using colony PCR (input pool probe). The remaining pooled bacterial cells are centrifuged, resuspended in phosphate buffered saline (PBS) to an inoculum dose of about 2×10⁷ cfu/ml, sonicated for 3 minutes, and injected into three fish. The fish are sacrificed at 7 days postinoculation and spleen, liver and kidney are harvested. The mutants that have reached and multiplied within these organs are recovered by plating homogenates of the organs onto laboratory medium. The recovered mutants from a given organ are combined and an aliquot is used for amplification using colony PCR (output pool probe). The products of the input and output pool amplification are used in a second PCR amplification using α-³²P dCTP to generate two radiolabeled probes. The amplified probes consist of a central variable region (the unique DNA tag) flanked by arms of invariable sequences which permit amplification of any tag using a defined set of primers. The arms are released by digestion with Hind III and the radiolabeled tags are used to probe replicate membranes from the master plasmid collection. Because of the complex structure of the mycobacterial cell wall and difficulties encountered in mycobacterial colony hybridization, in this example the amplified tags are used as probes to a dot blot containing the master plasmid collection. Hybridization to other forms of the master plasmid collection can, of course, be used. Tags from mutants that hybridize to the probe from the input pool (FIG. 7, membrane 1) but not to the probe from the output pool (FIG. 7, membrane 2) represent mutants which are unable to survive or compete in the fish model. Such mutants are designated as potential virulence mutants.

The pools of mutants recovered from different organs are kept separate, in order to characterize virulence mutants with regard to the organs examined. In some cases, mutations necessary for survival at different points in the pathogenesis of this organism can be identified, since the mechanisms necessary for survival in liver, spleen and kidney, or in other organs, may differ. The pools of mutants recovered form different fish are also kept separate. Mutants from two fish are used independently to produce an output pool probe and are independently hybridized to replica membranes to confirm reproducible identification of potential virulence mutants from a given experiment.

B. Confirming that the Mutants are Avirulent by Examining Individual Mutants in the Goldfish Model.

M. marinum transposon mutants that reproducibly hybridize to the input pool probe but not to the output pool probe are examined individually in the goldfish model. An inoculum dose of 10⁸ bacteria in 0.5 ml per fish is used to inoculate 3 fish per mutant. A control group of fish is simultaneously inoculated with M. marinum ATCC 927 (wild type) at the same dose as the mutants and with PBS as a negative control. The median survival time (MST) of goldfish inoculated with the wild type at this dose is 10 days. If the MST for a given mutant is greater than that of the wild type, this confirms that the mutant may have the transposon inserted into a virulence gene. When a mutant-inoculated fish survives for 35 days, it is sacrificed and examined for histopathology; and portions of the liver, spleen and kidney are homogenized and plated for colony counts. These mutants are then inoculated into fish to determine the LD₅₀. Three fish per mutant per dose are injected with 10⁸, 5×10⁷, or 10⁷ CFU bacteria. The LD₅₀ for each mutant is evaluated at 1 week postinoculation and calculated by the method of Reed and Meunch (1938. Am. J. Hyg. 27, 493-497). The LD₅₀ at 1 week for the wild type strain is 4.5×10⁸ CFU bacteria per fish. The LD₅₀, Competitive Index, and/or pathology for each mutant is compared to that of the wild type strain.

Competitive index: The competitive index may be used as a measure of the attenuation of a mutant with respect to a wild type strain. Mutant and wild type strains are mixed together in the inoculum. Animals are inoculated with the mixture and 1 week post-inoculation the animals are sacrificed. The liver and/or kidney of the animal is removed, homogenized, and the colony counts in the tissue are determined for both the mutant and wild type strains. The two strains are distinguished because the mutant is kanamycin resistant while the wild type is kanamycin sensitive. Mathematically, the competitive index is defined as the output ratio of mutant to wild type bacteria, divided by the input ratio of mutant to wild type bacteria. A mutant which has full virulence with respect to the wild type should not be out competed by the wild type and the competitive index should be 1.0.

Histopathology examinations: Portions of the liver, spleen and kidney along with peritoneum, heart, pancreas, or other organs evident to one of skill in the art, are fixed in 10% neutral buffered formalin for routine embedding in paraffin. Five μm thin sections of the paraffin fixed tissues are prepared with a rotary microtome (American Optical, Buffalo, N.Y.). After dewaxing, the sections are stained for acid fast bacilli with modified basic fuchsin stain and counterstained with methylene blue or stained with hematoxylin and eosn.

Colony counts in organ homogenates or the ability to induce granuloma formation: These parameters can identify virulence defects which are more subtle than one which causes the MST to change. Mutants identified in the screening protocol as failing to survive in vivo, but which fail to cause a significant change from wild type in MST when inoculated individually in fish, are further examined. For these experiments, an inoculum dose of 10⁷ CFU organisms are used, and animals are sacrificed at 2 week intervals for 4 weeks postinoculation. The liver, spleen and kidney are harvested and homogenized for analysis of colony counts. To examine the histopathology induced by mutants compared to wild type organisms, liver, spleen and kidney are harvested 8 weeks postinoculation for histopathology. Other organs which are evident to those of skill in the art can also be analyzed by colony counts or histopathology.

### Example 5

### Sequencing and Characterizing Regions Flanking the Transposons in the Virulence Mutants

Individual mutants confirmed in the goldfish model to be virulence mutants are examined by sequencing the nucleic acid flanking the site of insertion of the transposon. The sequence analysis can, of course, be performed before, simultaneously with, or after, a virulence defect has been confirmed.

A. Direct Sequencing of Flanking Regions

In a most preferred embodiment, chromosomal DNA is isolated from each mutant and cut with a restriction enzyme that cuts once within the transposon (in this example, with BamH1). Linkers bearing a predefined PCR primer site, designed and generated using routine, art-recognized methods, are ligated to the BamH1-cut ends; and PCR fragments are amplified, using as primers a first outward primer sequence specific for a portion of the transposon, and a second inward primer specific for the PCR primer site in the appended linker, to generate an “amplified PCR fragment”. In this example, a transposon-specific primer sequence is chosen based on the sequence of the inserted transposon, IS 1096. By “specific for,” as used herein, is meant that a primer (e.g., the first outward primer) is sufficiently complementary to a target (e.g., the transposon) to bind to it (hybridize; serve as a PCR primer) under selected high stringent conditions, but not to bind to other, unintended, nucleic acids. Southern analysis, in which the membrane to which the DNA has been transferred is probed with an α-³²P labeled aph (kanamycin resistance) gene, can be used to identify the size of the “amplified PCR fragment” from each mutant. For example, mutants 41.2, 80.1 and 86.1 shown in Example 9 have unique amplified PCR fragments, of 550, 200 and 600 bp, respectively. The amplified PCR fragments are sequenced directly, using as primers one or both of the primers used to generate them, or are cloned into a vector such as pGEM and sequenced using primers corresponding to vector sequences. Methods for probing gels and sequencing DNA are routine and conventional in the art.

In another embodiment, the chromosomal DNA is cut with an enzyme which does not cut within the transposon. A variety of enzymes can be tested until one which generates a DNA fragment of an appropriate size is identified. Here, Kpn I is used. The DNA is then ligated to create circular species and amplified by PCR using outward-facing primers complementary to the two ends of the transposon. In this way, the sequences which flank the insertion are amplified. These fragments are directly sequenced, using the same primers used to amplify the sequence.

B. Cloning and then Sequencing Flanking Regions

In another embodiment, the gene sequences interrupted by a transposon are cloned first and then sequenced. Procedures for the analysis of DNA, including isolating DNA, cloning it, manipulating it, and sequencing it, are routine and well-known in the art. In a preferred embodiment, genomic DNA is extracted from each virulence mutant, and is digested with one or more restriction enzymes (e.g., in this example, KpnI or BamHI) that provide genomic fragments of an appropriate size for cloning. The digested DNA is cloned into an appropriate plasmid, e.g., Bluescript II KS (Promega), or a low-copy plasmid such as pACYC184, in E. coli DH5α, by using an appropriate positive selection marker (e.g., kanamycin resistance). KpnI does not cut within the transposon, so digestion with Kpn I, followed by selection with kanamycin, results in cloning of the transposon along with flanking DNA. Bam HI cuts once within the transposon, so digestion with Bam HI, followed by selection with kanamycin, results in cloning of part of the transposon along with flanking DNA on one side of the transposon. Once cloned, the gene sequence interrupted (disrupted) by the transposon is determined by using outward primers based on the sequence of the transposon insertion sequence, in this example, IS1096 (See, e.g., McAdam et al (1995). Infec. Immun. 63, 1004-1012).

C. Comparison of Flanking Sequences to Known Databases

DNA sequences flanking each transposon (localized on one or on both sides of the site of transposon insertion) are compared with the use of the BLAST programs provided in the National Center for Biotechnology Information (NCBI) data base.

In order to identify M. tuberculosis homologues of M. marinum virulence genes, the flanking sequences are also compared to the Mycobacterium database, using the advanced Blast search program, as above.

A discussion of functional homologues and related virulence genes from M. tuberculosis which have been identified for 3 M. marinum mutants is presented in Example 9.

### Example 6

### Isolating and Characterizing Wild Type M. marinum Genes which Correspond to the Genes Disrupted by Transposons in Avirulent M. Marinum Mutants

Probes based on flanking M. marinum DNA sequences, characterized, e.g., as in Example 5, are generated and used to screen an M. marinum cosmid library (The construction of such a cosmid library is described below). For example, part or all of the “amplified PCR fragment” which is described in Example 5 is labeled and used as a hybridization probe. Conditions for specifically hybridizing a probe to a target nucleic acid (e.g., cosmid DNA) can be determined routinely by known methods in the art (see, e.g., Nucleic Acid Hybridization, a Practical Approach, B. D. Hames and S. J. Higgins, eds., IRL Press, Washington, 1985). It is preferred that hybridization probing is done under selected high stringent conditions to ensure that the gene, and not a relative, is obtained. Of course, conditions of any stringency can be employed. By “high stringent” is meant that the gene hybridizes to the probe (e.g., when the gene is immobilized on a filter) and the probe (which in this case is preferably about >200 nucleotides in length) is, e.g., in solution, and the immobilized gene/hybridized probe is washed in 0.1×SSC at 65° C. for 10 minutes. SSC is 0.15M NaCl/0.015M Na citrate. In general, “high stringent hybridization conditions” are used which allow hybridization only if there are about 10% or fewer base pair mismatches. As used herein, “high stringent hybridization conditions” means any conditions in which hybridization will occur when there is at least 95%, preferably about 97 to 100%, nucleotide complementarity (identity) between the nucleic acids. The corresponding cosmid is identified; and individual virulence genes are subcloned from the cosmid clone, using routine, conventional procedures in the art. The complete gene sequence is determined by routine, conventional methods.

Construction of an M. marinum cosmid library: An M. marinum genomic library in an E. coli—Mycobacteria shuttle cosmid (pYUB18) is constructed, using, e.g., methods disclosed in Jacobs, W. R. et al (1991). “Genetic Systems for Mycobacteria,” in Methods. Enzymol. 204,537-555. The pYUB18 vector has a unique BamHI site that can serve as the site of insertion of partial Sau3A-digested chromosomal DNA. Following in vitro packaging, the constructed libraries are transduced into cosmid in vivo packaging strains to permit amplification and efficient repackaging of recombinant cosmids into bacteriophage λ heads thus allowing for storage of the libraries as phage lysates.

### Example 7

### Isolating and Characterizing M. tuberculosis Genes which Correspond to M. marinum Virulence Genes

In order to identify an M. tuberculosis gene which corresponds to a particular M. marinum gene, an “amplified PCR fragment” from the M. marinum gene, such as that described in Example 5 or a fragment thereof, can be used to probe a cosmid library of M. tuberculosis. Most preferably, a probe based on the corresponding M. tuberculosis sequence, itself, is used. An M. tuberculosis cosmid library is constructed by routine methods. Hybridization is performed as described, e.g., in Example 6. Positive cosmid clones are identified and the hybridizing sequences subcloned and sequenced, using routine, conventional, methods in the art.

Alternatively, the sequence interrupted by the transposon in the M. marinum mutant can be directly compared to the M. tuberculosis genome using the advanced BLAST search by limiting the search to Mycobacterium.

Non-polar mutations in the M. marinum and M. tuberculosis strains can result in deletions of about >50% of the structural gene, e.g., about >75%, >90%, >95%, or 100%. A goal of mutation of virulence genes in vaccine strains is to disrupt the function of the virulence gene. Such a large deletion will achieve this goal, often resulting in loss of an important antigenic determinant.

Well-defined mutations can be introduced into a cloned M. tuberculosis gene, using the methods described herein, e.g., for generating site-specific mutations (such as deletions, e.g., in phase deletions) in M. marinum genes. The mutations can then be introduced into the M. tuberculosis genome by homologous recombination. In a most preferred embodiment (as disclosed, e.g., in Balasubramanian, V. et al (1996). J. Bacteriol. 178, 273-279, and Reyrat, J. et al (1995). PNAS 92, 8768-8772), the recombination is performed with long linear recombination substrates containing the mutated gene (virulence gene::aph) on a DNA fragment (>40 kb). This fragment is electroporated into the H37Rv strain of M. tuberculosis selecting for kanamycin resistance. Chromosomal DNA from the parent H37Rv strain and the kanamycin-resistant transformants are digested with KpnI and probed with a KpnI fragment containing the virulence gene::aph fragment. The strains containing the disrupted allele show a signal from a fragment which is 1.3-kb greater (aph gene) than the hybridizing fragment from the wild type gene clone (control). These mutant strains can be tested, e.g., in the guinea pig infection model (See, e.g., Collins, D. M. et al (1995). PNAS 92, 8036-8040).

Alternatively, allelic exchange can be performed using ts-sacB vectors (see, e.g., Pelicic et al. (1997). PNAS 94, 10955-10960). The virulence gene::aph construct is inserted into pJM10, a ts-sacB E. coli—Mycobacteria vector containing the kanamycin resistance gene for selection. The plasmid is introduced into the H37Rv strain of M. tuberculosis by electroporation with selection initially at 32° C. on 7H10-kanamycin. Transformants are selected, grown in liquid culture, and then plated at 39° C. on 7H10-kanamycin+2% sucrose plates. Transformants obtained on the counterselective plates represent allelic exchange mutants.

M. tuberculosis vaccine strains can be produced by using a protocol to construct unmarked deletion mutations which requires a two-step allelic exchange. A suicidal recombination plasmid containing the nonpolar deletion mutant of the gene of interest is electroporated into cells, and primary recombinants are selected upon kanamycin (encoded in the backbone of the suicide plasmid) medium. Since the plasmid cannot replicate, any kanamycin-resistant clones must integrate the plasmid into the chromosome by a single-crossover event. Because of the presence of the sacB gene on the suicide vector backbone, the kan^{r} clones are also sensitive to sucrose (Suc^{s}). [The Suc^{s} phenotype distinguishes from spontaneous kan^{r} clones as these will be Suc^{r} because they did not result from integration of the plasmid and sacB gene into the chromosome.] Plasmid integration at the desired locus results in a tandem duplication of the cloned region with the vector DNA in the middle. One such clone is then grown to saturation in medium without antibiotics, during which time individuals within the population undergo a second homologous recombination event between the duplicated regions. In this event, the plasmid vector is lost along with the aph (kan^{r}) and sacB genes, leaving behind either the wild-type or the mutant allele, depending upon which side of the mutation the second recombination event occurred. This second recombination event occurs at low frequency; thus, there must be a selection for the desired secondary recombinants. To select these clones, one takes advantage of the loss of the sacB gene; any clone losing the plasmid is now sucrose resistant (Suc^{r}). The culture is plated on medium containing sucrose to kill any clones that did not undergo a second recombination event. The sucrose-resistant clones are then screened for kanamycin sensitivity and the targeted gene amplified by PCR to confirm the deletion mutation (smaller amplified product compared to wild-type gene). Spontaneous sacB mutations will also have a sucrose resistant phenotype, but these can be distinguished from the actual second recombination event desired because these clones are kan^{r}.

### Example 8

### Complementation Assays

A candidate virulence gene is reintroduced into a transposon mutant on a low copy number E. coli—mycobacteria shuttle vector (pYUB213Δkm) (Ramakrishnan, L. et al (1997). J. Bacteriol. 179, 5862-5868) to determine whether the cloned gene complements the virulence defect in the goldfish model. This plasmid is a derivative of pMV262 (Stover, C. K. et al (1991). Nature 351, 456-460) with a bleomycin resistance gene for selection. Bacteria are recovered from those fish in which the virulence defect has been complemented, and analyzed for bleomycin and kanamycin resistance to confirm that the complementing plasmid is present.

Some cloned virulence gene candidates may fail to complement the virulence defect in the fish model because of, e.g., instability of the cosmid clone, polar effects in the original mutation, requirement for a cluster of genes surrounding the interrupted gene, or toxic effects associated with overexpression of genes from multicopy plasmids. In order to overcome these problems, several alternative approaches can be used.

One approach is to utilize an integrating E. coli—mycobacterial shuttle vector, pMV361 (Stover, C. K. et al (1991). Nature 351, 456-460). The vector integrates in a site-specific manner into the chromosomal attB site. This site is in a well-conserved part of the mycobacterial genome and has been identified in BCG, M. smegmatis, M. bovis, M. chelonei, M. leprae, M. phlei, and M. tuberculosis. Prior to the use of this vector in M. marinum, the presence of the attB site in M. marinum is confirmed by Southern blot analysis of M. marinum chromosomal DNA digested with BamHI using a radiolabeled 1.7-kb Sal I attB fragment from M. smegmatis. In order to use this vector in mutants which contain the kanamycin resistance gene, the vector is modified to delete the kanamycin gene and to insert the bleomycin gene as was done, e.g., with the construction of pYUB213Δkm (Ramakrishnan, L. H. et al (1997). J. Bacter. 179, 5862-5868). Using an integrating vector eliminates the possible instability seen with extrachromosomal plasmid maintenance in vivo (the integrated vector is stably maintained even without antibiotic selection), and the toxic effects associated with multicopy plasmids are reduced or eliminated since integration results in a single copy of the gene in the chromosome. To address the issue that the original transposon insertion phenotype was due to a polar effect on a downstream gene or that a cluster of genes is required for complementation, larger fragments of the original cosmid clone can be inserted into the integrating plasmid.

Another approach is to construct by allelic exchange specific chromosomal mutations in the identified virulence genes. Methods for using long linear recombination substrates for allelic exchange are provided, e.g., in Balasubramanian, V. et al (1996). J. Bacteriol. 178, 273-279. Other methods for homologous recombination are found, e.g., in Aldovini, A. R. et al (1993). J. Bacteriol. 175, 7282-7289; Norman, E. et al (1995). Mol. Microbiol. 16, 755-760; Baulard, A. et al (1996). J. Bacteriol. 178, 3091-3098; Marklund, B. I. et al (1995). J. Bacteriol. 177, 6100-6105; Ramakrishnan, L. et al (1997). J. Bacteriol. 179, 5862-5868; and Parelka M. S. et al. (1999) J. Bacteriol. 181, 4780-4789. These specific mutations allow the creation of non-polar mutations in the virulence genes.

### Example 9

### Identification and Characterization of Thirteen M. tuberculosis Virulence Genes

DNA regions flanking transposon insertion points for 13 mutants were amplified by inverse PCR and sequenced. Predicted amino acid sequences from all six reading frames of the DNA sequences obtained were subjected to similarity search of the nr database, using the NCBI BLAST program. The nr database includes, e.g., all non-redundant GenBank CDS translations, PDB, SwissProt, PIR and PRF sequences. An advanced BLAST search determined whether a homologous protein sequence was present in the Mycobacterium tuberculosis genome.

Gene 41.2

A sequence of the flanking region of M. marinum mutant 41.2 is as follows:

(SEQ ID NO:4)

5′-ACGACGGGACAGATGGGTCCCCGGATGGTCTACACCGAGACCAAACT
GAACTCGTCGTTCTCCTTCGGCGGGCCCAAGTGTCTGGTGAAGGTGATCC
AAAAACTGTCCGGGTTGAGCATCAACCGGTTCATCGCCATCGACTTCGTC
GG-3′

This can be translated in the third reading frame to the following protein sequence:

(SEQ ID NO:5)

| | |
|---|---|
| 1 | TTGQMGPRMVYTETKLNSSFSFGGPKCLVKVIQKLSGLSINRFI |
| 51 | AIDFV |

A longer sequence of the open reading frame disrupted in mutant 41.2 is shown elsewhere herein.

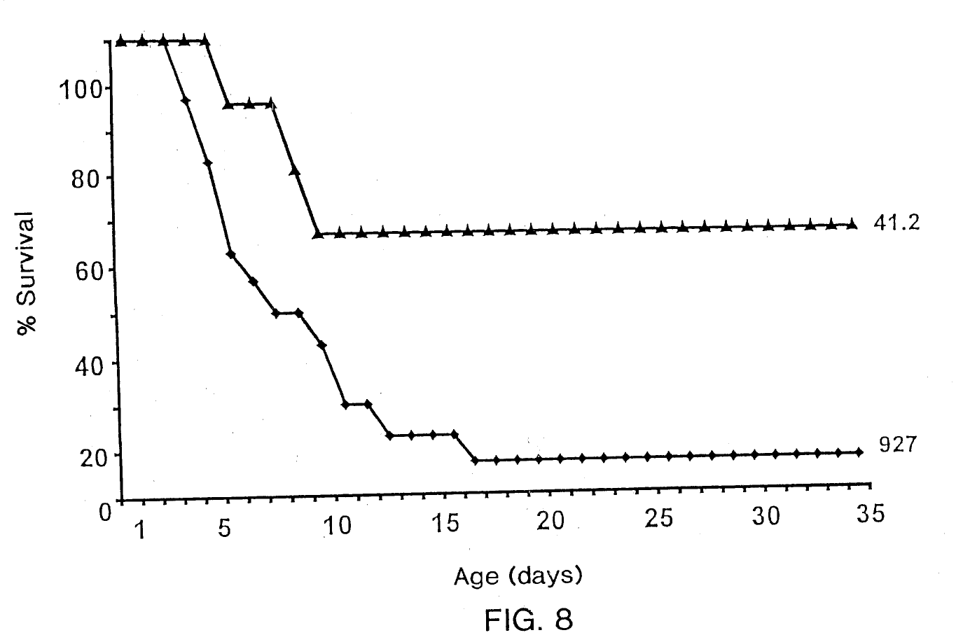

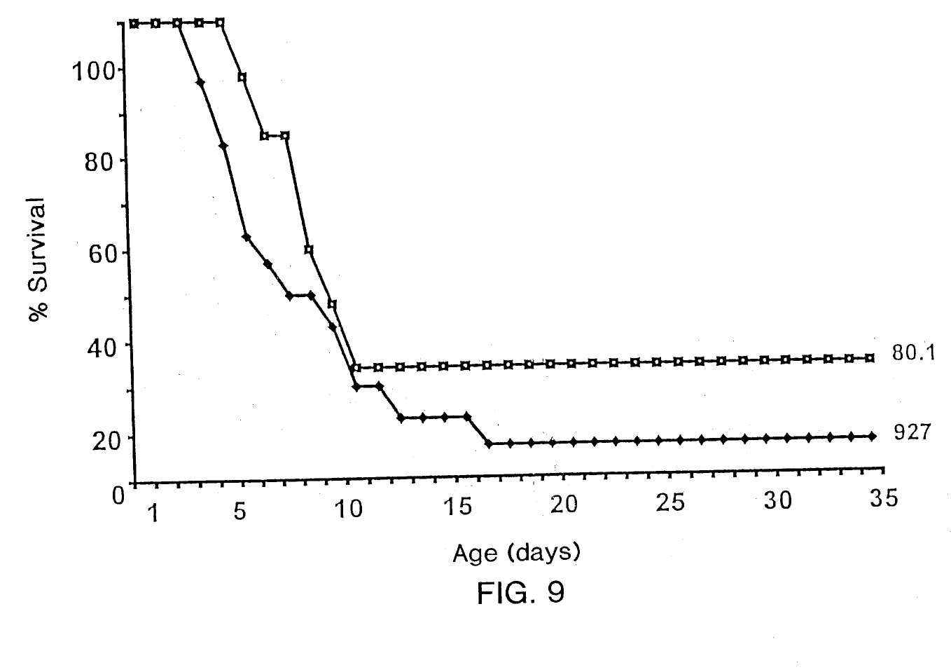

The mutant (41.2), when tested individually in the goldfish model, exhibits attenuated virulence as compared to the wild type organism (See FIG. 8).

The gene interrupted in the attenuated mutant has been characterized by sequence analysis. Using the mycobacterium database, a functional homologue of this gene has been identified in M. tuberculosis (emb|CAA17628| (AL022004); (Rv0822c). Using the general genomic database, the gene has been shown to be most closely related to gene emb|CAA20411|; (AL031317), a transcriptional regulator of Streptomyces coelicolor which belongs to the AraC family of transcriptional regulators. This suggests that the gene identified as interrupted in mutant 41.2 is a transcriptional regulator belonging to the AraC family.

The proteins belonging to this family have at least three main regulatory functions in common: carbon metabolism, stress response, and pathogenesis. (See, e.g., Gallegos, M-T et al (1997). Microbiology and Molecular Biology Reviews 61 393-410). Certain of these regulatory proteins are involved in the production of virulence factors in infections of plants or mammals. These regulatory factors have been found in microbes that colonize either the gastrointestinal, respiratory, or genitourinary tracts. These proteins are involved in stimulation of the synthesis of proteins that play a role in adhesion to epithelial tissues, components of the cell capsule, and invasins. Some members of the family control the production of other virulence factors. Some regulators are involved in the response to stressors, including oxidative stress and transition from exponential growth to the stationary phase. Without wishing to be bound by any particular mechanism, these observations suggest that the role of this gene in M. tuberculosis pathogenesis may be in invasion of the macrophage, survival in the macrophage (oxidative stress) or in transition to the latent state of tuberculosis (transition from exponential to stationary phase).

Gene 86.1

The sequence of the flanking region of M. marinum mutant 86.1 is as follows:

(SEQ ID NO:8)

5′-TCATCGCTAACCGGTTGAGCTACCGCCCGCACAGCGTGCCCATCATC
TCCAACCTGACCGGCTCACTTGCCACAGTCGAGCAACTCACATCGCCCCG
CTATTGGGCACAGCATGTACGGGAGCCAGTGCGGTTTCATGACGGCGTTA
CCGGCTTGTTGGCAGGCGGAGAACA-3′

This can be translated in the third reading frame to the following protein sequence:

| | |
|---|---|
| 1 I A N R L S Y R P H S V P I I S N L T G S L A T V E Q L T S P R | (SEQ ID NO:9) |
| Y W A Q H V R E P V R F H D G V T G L L A G G E. | |

A longer sequence of the open reading frame disrupted in mutant 86.1 is shown elsewhere herein.

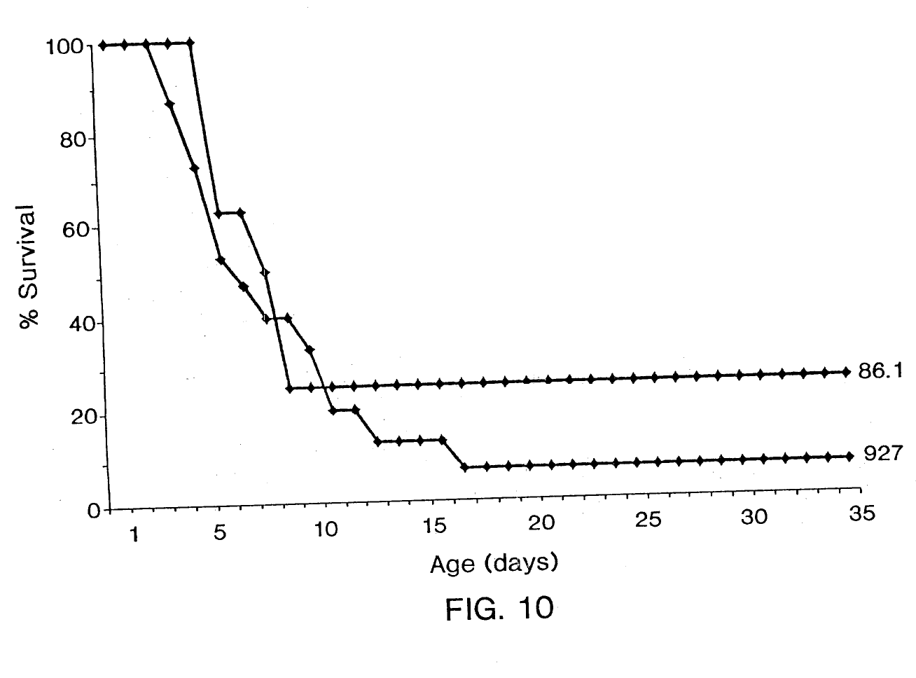

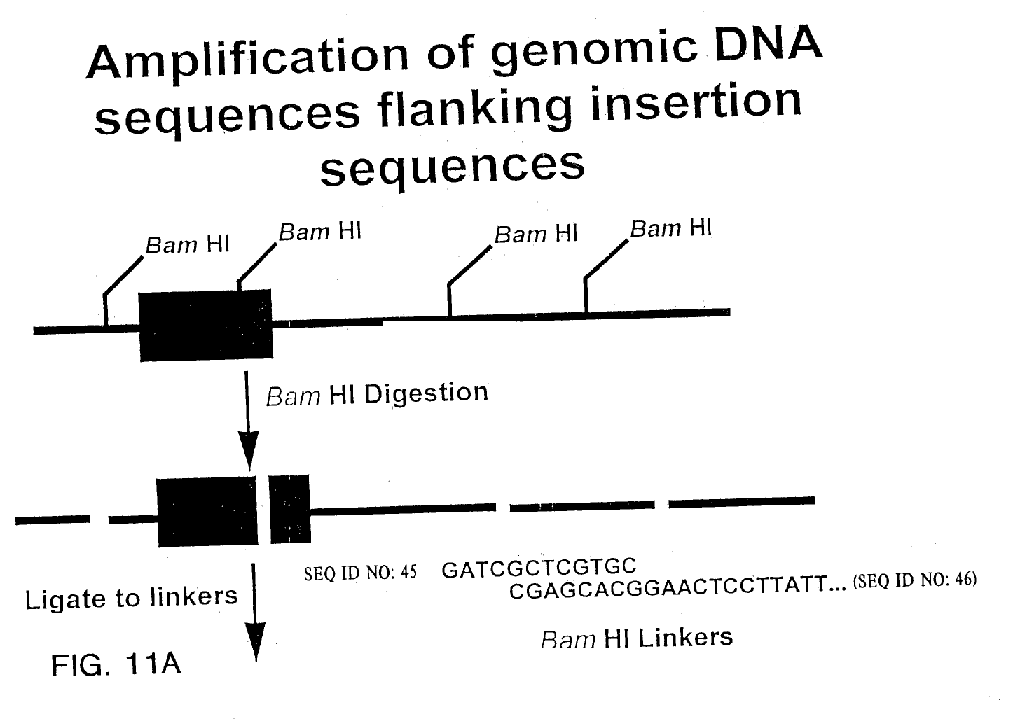

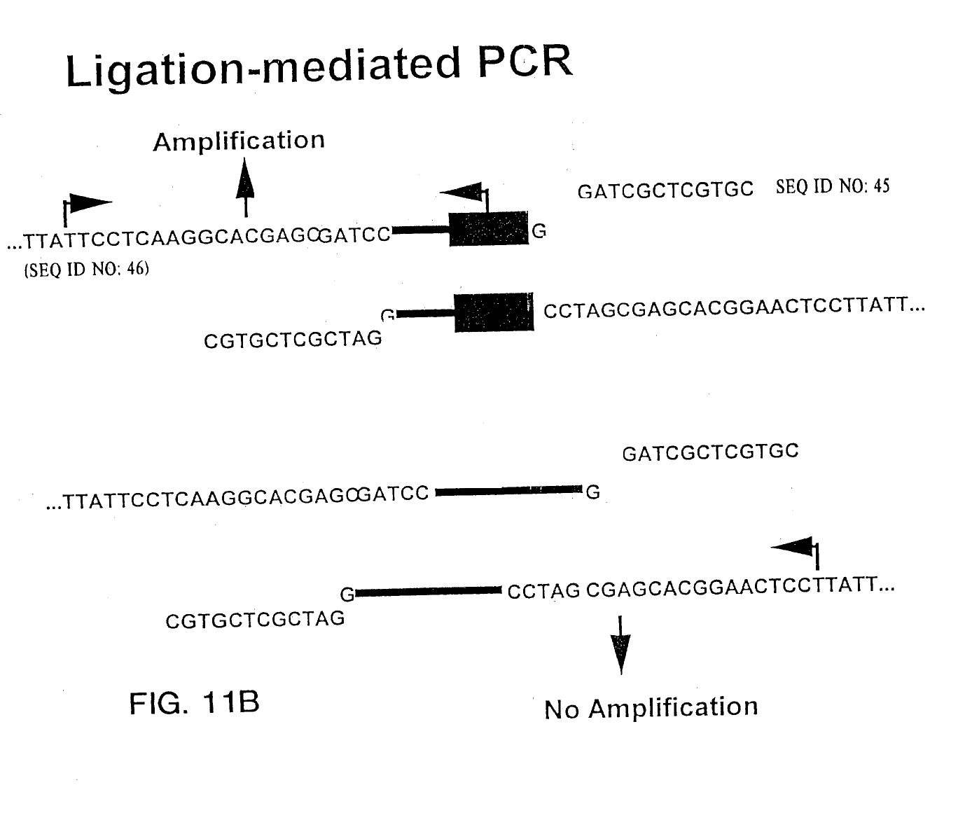

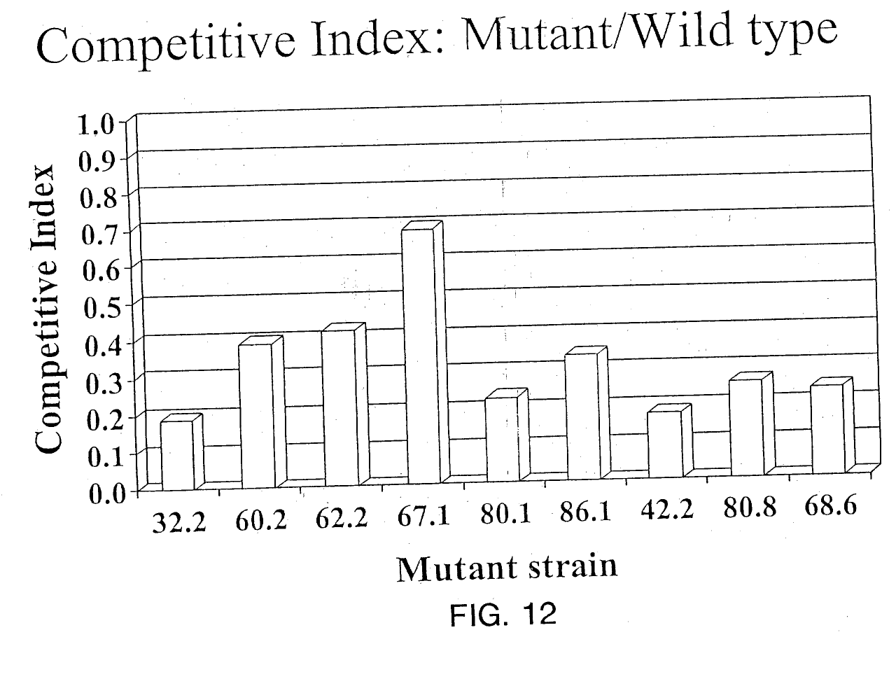

The mutant (86.1), when tested individually in the goldfish model, exhibits attenuation in virulence as compared to the wild type organism (See FIGS. 10 and 12).

Gene 67.1

The sequence of the flanking region of M. marinum mutant 67.1 is as follows:

(SEQ ID NO:13)

GGTCGAAGACTATCGGTATGCTCCATAGCGTTCCGTCGGGAAGCTGCATG
TTGTCAAGGGTTTCGTCGACCTCTCGGCGACCCATGAATCCCGATAGTGG
CGTGAAGAAACCGTACGAGATGCTGATCACCTCGTGGGCGGTCGCCTTCG
ATATCGGGATGCGCACCAATCCCTCAATCCGGCCGGCCACGTTTTCCCTT
TCCACCCTGTCGACGAGTGGGTGTCCGTTATGGCCTAAATAATCCATCTT
GCTGCCTCTTTCTGAAATCGAATTTATTACTATCG

This can be translated in the six reading frames to the following protein sequences:

| DNA: | GGTCGAAGACTATCGGTATGCTCCATAGCGTTCCGTCGGGAAGCTGCATGT | (SEQ ID NO:13) |
|---|---|---|
| +3: | S K T I G M L H S V P S G S C M L | (SEQ ID NO:14) |
| +2: | V E D Y R Y A P * R S V G K L H V | (SEQ ID NO:15) |
| +1: | G R R L S V C S I A F R R E A A C | (SEQ ID NO:16) |
| DNA: | TGTCAAGGGTTTCGTCGACCTCTCGGCGACCCATGAATCCCGATAGTGGCG | |
| +3: | S R V S S T S R R P M N P D S G V | |
| +2: | V K G F V D L S A T H E S R * W R | |
| +1: | C Q G F R R P L G D P * I P I V A | |
| DNA: | TGAAGAAACCGTACGAGATGCTGATCACCTCGTGGGCGGTCGCCTTCGATA | |
| +3: | K K P Y E M L I T S W A V A F D I | |
| +2: | E E T V R D A D H L V G G R L R Y | |
| +1: | * R N R T R C * S P R G R S P S I | |
| DNA: | TCGGGATGCGCACCAATCCCTCAATCCGGCCGGCCACGTTTTCCCTTTCCA | |
| +3: | G M R T N P S I R P A T F S L S T | |
| +2: | R D A H Q S L N P A G H V F P F H | |
| +1: | S G C A P I P Q S G R P R F P F P | |
| DNA: | CCCTGTCGACGAGTGGGTGTCCGTTATGGCCTAAATAATCCATCTTGCTGC | |
| +3: | L S T S G C P L W P K * 5 I L L P | |
| +2: | P V D E W V S V M A * I I H L A A | |
| +1: | P C R R V G V R Y G L N N P S C C | |
| DNA: | CTCTTTCTGAAATCGAATTTATTACTATCG | |
| +3: | L S E I E F I T I | |
| +2: | S F * N R I Y Y Y | |
| +1: | L F L K S N L L L S | |
| DNA: | CGATAGTAATAAATTCGATTTCAGAAAGAGGCAGCAAGATGGATTATTTAG | (SEQ ID NO:17) |
| −1: | R * * * I R F Q K E A A R W I I * | (SEQ ID NO:18) |
| −2: | D S N K F D F R K R Q Q D G L F R | (SEQ ID NO:19) |
| −3: | I V I N S I S E R G S K M D Y L G | (SEQ ID NO:20) |
| DNA: | GCCATAACGGACACCCACTCGTCGACAGGGTGGAAAGGGAAAACGTGGCCG | |
| −1: | A I T D T H S S T G W K G K T W P | |
| −2: | P * R T P T R R Q G G K G K R G R | |
| −3: | H N G H P L V D R V E R E N V A G | |
| DNA: | GCCGGATTGAGGGATTGGTGCGCATCCCGATATCGAAGGCGACCGCCCACG | |
| −1: | A G L R D W C A S R Y R R R P P T | |
| −2: | P D * G I G A H P D I E G D R P R | |
| −3: | R I E G L V R I P I S K A T A H E | |
| DNA: | AGGTGATCAGCATCTCGTACGGTTTCTTCACGCCACTATCGGGATTCATGG | |
| −1: | R * S A S R T V S S R H Y R D S W | |
| −2: | G D Q H L V R F L H A T I G I H G | |
| −3: | V I S I S Y G F F T P L S G F M G | |
| DNA: | GTCGCCGAGAGGTCGACGAAACCCTTGACAACATGCAGCTTCCCGACGGAA | |
| −1: | V A E R S T K P L T T C S F P T E | |
| −2: | S P R G R R N P * Q H A A S R R N | |
| −3: | R R E V D E T L D N M Q L P D G T | |
| DNA: | CGCTATGGAGCATACCGATAGTCTTCGACC | |
| −1: | R Y G A Y R * S S T | |
| −2: | A M E H T D S L R | |
| −3: | L W S I P I V F D | |

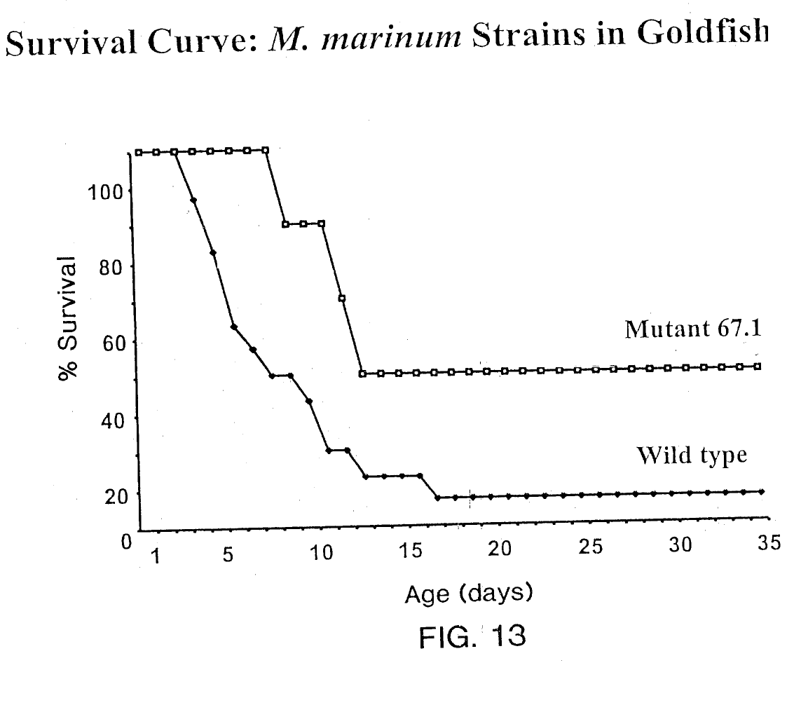

The mutant (67.1), when tested individually in the goldfish model, exhibits attenuated virulence as compared to the wild type organism (See FIGS. 12 and 13).

The gene interrupted in the attenuated mutant has been characterized by sequence analysis, as described above for mutant 41.2.

Based on the sequence analysis to the entire genomic database, the gene identified as interrupted from mutant 67.1 is a sulfate adenylyltransferase with homology to diverse organisms including Pyrococcus abyssi, Synechocystis sp., and Bacillus subtilis. The homology is in the −3 reading frame of the translated gene product and shows 27-40% identity (51-62% similar). The homology noted to the sulfate adenylyltransferase enzymes suggests that mutant 67.1 is attenuated in its ability to respond to sulfate starvation as this enzyme is required for growth in defined synthetic medium with sulfate as a sulfur source. This suggests that in the animal host a sulfur source is limiting and thus interruption of this gene attenuates growth of the organism in the animal host.

Gene 39.2

The sequence of the flanking region of M. marinum mutant 39. is as follows:

(SEQ ID NO:23)

GATCCGCTGGACGGCACCAAAGAATTCATCAAGGGCAGCGATGAGTTCAC
CGTCAACATCGCCCTGGTCGAGAACCAGGAACCCATTCTCGGGGCAATCT
ACGGTCCAGCGAAGCAACTTCTGCACTACGCGGCCAAAGGGGCT

This can be translated in the +1 reading frames to the following protein sequence:

(SEQ ID NO:43)

| | |
|---|---|
| 7 | ctggacggcaccaaagaattcatcaagggcagcgatgagttcacc |

(SEQ ID NO:24)

| | |
|---|---|
| | L D G T K E F I K G S D E F T |
| 52 | gtcaacatcgccctggtcgagaaccaggaacccattctcggggca |
| | V N I A L V E N Q E P I L G A |
| 97 | atctacggtccagcgaagcaacttctgcactacgcggccaaaggg |
| | I Y G P A K Q L L H Y A A K G |
| 142 | gct 144 |
| | A |

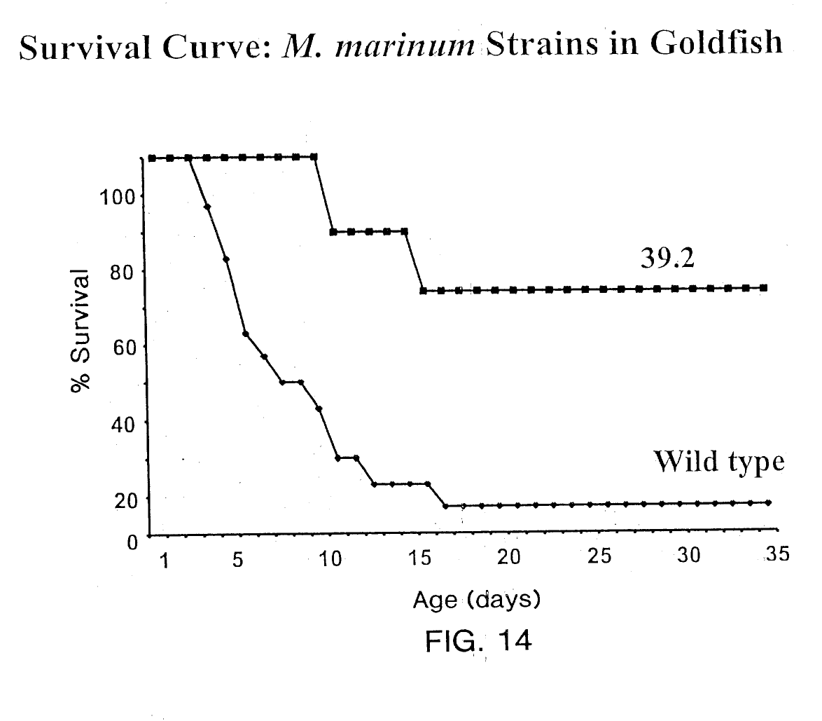

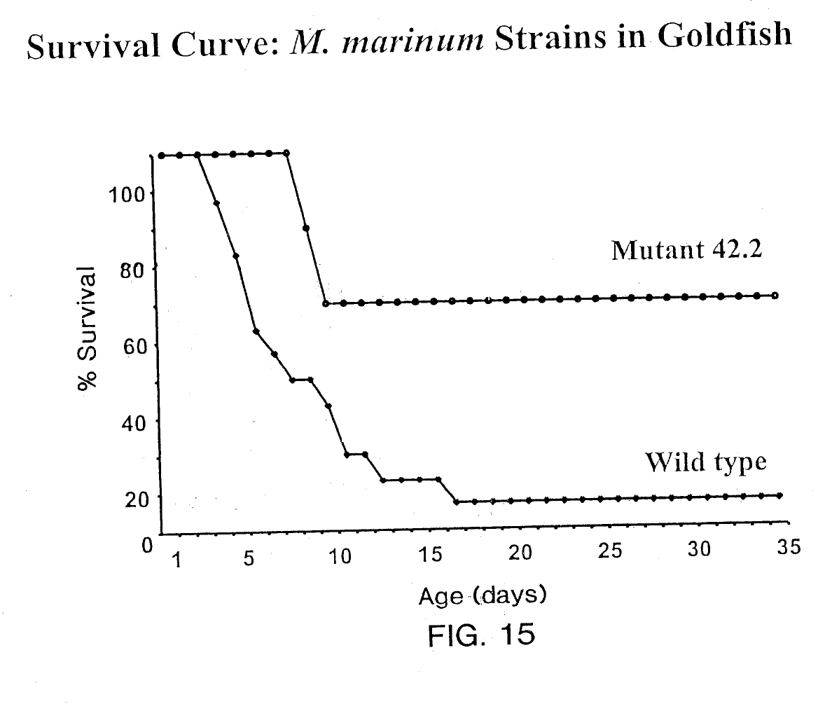

The mutant (39.2), when tested individually in the goldfish model, exhibits attenuation in virulence as compared to the wild type organism (See FIG. 14).

The gene interrupted in the attenuated mutant has been characterized by sequence analysis, as described above for mutant 41.2. Using the mycobacterium database, a functional homologue of this gene has been identified in M. tuberculosis (emb|CAB06277.1|Z8386| hypothetical protein Rv3137). This homologue, in the +1 frame, with an identity 43% (similarity of 63%), is a probable inositol monophosphate phosphatase, because it contains an inositol monophosphatase family signature sequence. It is related to the cysQ proteins identified in the whole database search described below, which also belong to the inositol monophosphatase family.

Based on a sequence analysis to the entire genomic database, the gene identified as interrupted from mutant 39.2 is a structural protein of an ammonium transport system (also known as a cysQ gene). This protein affects the pool of 3′-phosphoadenosine -5′-phosphosulfate in the pathway of sulfite synthesis. The identity is in the +1 reading frame of the translated gene product and is 53-65% identical (63-82% similar). The homology noted suggests that mutant 39.2 is attenuated in its ability to respond to sulfate starvation as this enzyme is required for growth in defined synthetic medium with sulfate as a sulfur source. This suggests that in the animal host a sulfur source is limiting and thus interruption of this gene attenuates growth of the organism in the animal host.

Gene 114.7

The sequence of the flanking region of M. marinum mutant 114.7 is as follows:

(SEQ ID NO:25)

AGCCGTATTTCGCCATTGAGAGTTGGGGTCTTGAGATCGGCACTGGAAGG
GGACAGCGTGCTATTGCCTCTTGGTCCGCCCTTGCCACCTGATGCTGTGG
CGGCTAAACGGGGTGAGTCGGGGCTGCTCTGCGGCTTGTCGGTTCCGCTC
AGCTGGGGTACGGCCGTTCCGCCGGATGACTACNACCATTGGGCACCGGA
GCCTGAAGAAGGCGCCGAGGCCGTGGTCGAAGAAAACGTGGATGCGGCAG
CTGCCGGTACCGACGAGTGGGACGAGTGGGCGGAATGGAGGGAGTGGGAG
GCAGCAAATGCCCGAACCTCATTTTCGAGATGCCCCGTACCAGCAGCCGT
GATACCCGAACTCGCCGGCGGCCGGTTGAGA

This can be translated in the +1 reading frames to the following protein sequence:

(SEQ ID NO:44)

| | |
|---|---|
| 16 | ttgagagttggggtcttgagatcggcactggaaggggacagcgtg |

(SEQ ID NO:26)

| | |
|---|---|
| | L R V G V L R S A L E G D S V |
| 61 | ctattgcctcttggtccgcccttgccacctgatgctgtggcggct |
| | L L P L G P P L P P D A V A A |
| 106 | aaacggggtgagtcggggctgctctgcggcttgtcggttccgctc |
| | K R G E S G L L C G L S V P L |
| 151 | agctggggtacggccgttccgccggatgactacnaccattgggca |
| | S W G T A V P P D D Y X H W A |
| 196 | ccggagcctgaagaaggcgccgaggccgtggtcgaagaaaacgtg |
| | P E P E E G A E A V V E E N V |
| 241 | gatgcggcagctgccggtaccgacgagtgggacgagtgggcggaa |
| | D A A A A G T D E W D E W A E |
| 286 | tggagggagtgggaggcagcaaatgcccgaacctcattttcgaga |
| | W R E W E A A N A R T S F S R |
| 331 | tgccccgtaccagcagccgtgatacccgaactcgccggcggccgg |
| | C P V P A A V I P E L A G G R |
| 376 | ttgaga 381 |
| | L R |

The mutant (114.7), when tested in pools in the goldfish model, appears to exhibit attenuation in virulence as compared to the wild type organism.

The gene interrupted in the attenuated mutant has been characterized by sequence analysis. Using either the mycobacterium or the general genomic database, a functional homologue of this gene has been identified in M. tuberculosis (pir E70662); (Rv2348c). The homology is in the +1 reading frame, with an identity of 82% (similarity 84%), to a hypothetical protein of M. tuberculosis. This protein is of unknown function as it has no known homology to any other sequence in the database. This gene is a virulence gene in M. marinum and M. tuberculosis.

### Example 10

### Identification and Characterization of More M. marinum Virulence Genes, and Corresponding M. tuberculosis Virulence Genes

Identification of transposon insertion sites using Ligation Mediated PCR (LMPCR). LMPCR was performed as described by Prod'hom et al., except BamHI linkers were used; PCR products were analyzed by electrophoresis on a 2% agarose gel and purified (Qiaex II, Qiagen) per manufacturer's instructions. The products were sequenced using primers T89 (5′-TTTGAGCTCTACACCGTCAAGTGCGAAG-3′) (SEQ ID NO: 47) and T100 (5′-TAGCTTATTCCTCAAGGCACGAGC-3′) (SEQ ID NO: 48) complementary to sequence in the insertion element of the transposon.

Identification of transposon and flanking DNA sequences by cosmid cloning of the STM mutants. Chromosomal DNA from each mutant was prepared as described by Belisle et al. (1988). Methods Mol Biol 101, 31-44], partially digested with Sau3AI (Invitrogen) and 30 kilobase fragments were purified by chloroform/phenol/isoamyl alcohol (25:24:1) and ethanol precipitation. The cosmid vector pHC79 (Hohn et al. (1980). Gene 11, 291-298) was digested with BamHI, and dephosporylated with shrimp alkaline phosphatase. The partially digested DNA was ligated to the cosmid vector and packaged using the Gigapack® III XL Packaging Extract according to manufacturer's instructions (Stratagene, La Jolla, Calif.). E. coli strain VCS257 (Stratagene) was transformed with packaged phage, and clones containing the transposon with the flanking DNA were selected on Luria agar supplemented with 50 μg/ml kanamycin. Kanamycin resistant clones were grown in LB and cosmid DNA was isolated using the Concert™ High Purity Plasmid Maxiprep System (Invitrogen) according to manufacturer's instructions. Cosmids were sequenced using primers T340 (5′-GCTCTTCCTCTTGCTCTTCC-3′) (SEQ ID NO: 49) and T343 (5′-TCCATCATCGGAAGACCTCG-3′) (SEQ. ID NO. 50) which were designed to sequence at either ends of the transposon moving outward into the flanking M. marinum DNA sequence.

Sequencher™ (Gene Codes Corporation, Ann Arbor, Mich.) was used to assemble the sequences from either ends of the transposon. Short stretches of DNA sequence representing the transposon insertion repeats were identified and removed. The average size of the sequence obtained for each mutant was 1200 base pairs. This sequence was analyzed for open reading frames (ORFs) using the network program ORF finder (NCBI). The ORF interrupted by the transposon in the M. marinum mutant was identified and compared to the M. tuberculosis genome using the advanced BLAST search by limiting the search to Mycobacterium.

The DNA sequence of the open reading frame interrupted in M. marinum mutant 32.2 and its translated protein sequence is as follows:

(SEQ ID NO:51)

| | |
|---|---|
| 1124 | atgagccagctgaatccgcccggtcccagccagttgtcgtttgtg |

(SEQ ID NO:52)

| | |
|---|---|
| | M S Q L N P P G P S Q L S F V |
| 1079 | ctgaceggcaatccgaacaaccccgacggcggcgtcctcgaacgc |
| | L T G N P N N P D G G V L E R |
| 1034 | ttcaacggtctttacctcccgattgtggatgtgttgttcaatggc |
| | F N G L Y L P I V D V L F N G |
| 989 | gcgaccccgccggattcgccctatcccaccgccatttacaccgcc |
| | A T P P D S P Y P T A I Y T A |
| 944 | caatacgacggcatagccaacttcccgcgctacccgctcaatgtg |
| | Q Y D G I A N F P R Y P L N V |
| 899 | gtgtcggacgtgaacgcgataatgggcttcctttatgacgagcac |
| | V S D V N A I M G F L Y D E H |
| 854 | tactacgcgggcctgacatcggacccgaacgccatagactccggg |
| | Y Y A G L T S D P N A I D S G |
| 809 | cccaccgttgccaacgcggtgcagctgccgacctcgcccggctac |
| | P T V A N A V Q L P T S P G Y |
| 764 | accggcaataccgagtactacatggtcctggcccagcatctaccg |
| | T G N T E Y Y M V L A Q H L P |
| 719 | cttaccgacccgcttcgtcagatcccgtacgtgggaacacccatc |
| | L T D P L R Q I P Y V G T P I |
| 674 | gccgatctgatccagccctcgctgcgggtgatcgtggacttgggc |
| | A D L I Q P S L R V I V D L G |
| 629 | tacagcgattacgcgattacggaccgggacagaactatgcggaca |
| | Y S D Y A I T D R D R T M R T |
| 584 | tccccacccccgcctcactgttctcgctgcccaacccgctcgcgg |
| | S P P P P H C S R C P T R S R |
| 539 | tga 537 |

The gene interrupted in the attenuated mutant has been characterized by sequence analysis. Using the Mycobacterium database, functional homologues of this gene have been identified in M. tuberculosis [pir E70839 emb CAA17314.1 (AL021927) (Rv0159c), ref NP 214673.1; pir F70839 emb CAA17315.1 (AL021927) (Rv0160c) ref NP 214674.1]. The homology with the M. tuberculosis homologue Rv0159c is 62% identity, 74% similarity and with Rv0160c is 59% identity, 71% similarity.

This is a gene encoding a member of the PE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 42.2 and its translated protein sequence is as follows:

(SEQ ID NO:53)

| | |
|---|---|
| 1159 | cacaacgtggqtgtggccaatgccggcttcaacaatttcggtttc |

(SEQ ID NO:54)

| | |
|---|---|
| | H N V G V A N A G F N N F G F |
| 1114 | gccaatacggqcagcaacaacatcgggattgggctcagtggggat |
| | A N T G S N N I G I G L S G D |
| 1069 | gggcaggtcgggttcggggcgctgaactcgggcaccggcaatatc |
| | G Q V G F G A L N S G T G N I |
| 1024 | gggttgttcaactccggcaccgacaacatcgggttgttcaattcg |
| | G L F N S G T D N I G L F N |
| 979 | gggacgggcaacttcgggatcggcaactccggtgactacaacact |
| | G T G N F G I G N S G D Y N T |
| 934 | ggcatcggcaacgcgggcgccaccaacaccggcctgttgaatgcg |
| | G I G N A G A T N T G L L N A |
| 889 | ggtctggtcaacaccggtgtgggcaacgcgggcaactacaactcc |
| | G L V N T G V G N A G N Y N S |
| 844 | ggtggcttcaacgccgggcacaccaacaccggcagcttcaactcc |
| | G G F N A G H T N T G S F N S |
| 799 | ggtgactacaacaccggctacctcaacccgggtaactacaacacc |
| | G D Y N T G Y L N P G N Y N T |
| 754 | ggtctggccaacagcggcgacgtcaacaccggtgcgttcatctcc |
| | G L A N S G D V N T G A F I S |
| 709 | ggcaattacagcaacggcgccttctggcgcggcgaccaccagggc |
| | G N Y S N G A F W R G D H Q G |
| 664 accggaatttcttactcggtcacgatcccagcgattccgatcaat | |
| | T G I S Y S V T I P A I P I N |
| 619 atcaacgagacttatagtcttatagtctggacataccgtttaccg | |
| | I N E T Y S L I V W T Y R L P |
| 574 | aagacatcggacccaggtccattgcaagctttgtcattccccggc |
| | K T S D P G P L Q A L S F P G |
| 529 | aatcggtcaccgtga 515 |
| | N R S P * |

The gene interrupted in the attenuated mutant has been characterized by sequence analysis. Using the Mycobacterium database, functional homologues of this gene have been identified in M. tuberculosis [refNP214819.1; embCAB09593.1; pirB70524 (Rv0305c); ref NP 214869.1, emb CAB08587.1, pir D70575 (Rv0355c); refNP214818.1; pirA70524; embCAB09611.1 (Rv0304c); refNP217864.1; pirB70969; embCAA15732.1 (Rv3347c)]. The homology with the M. tuberculosis homologue Rv 0305c is 60% identity, 74% similarity, with Rv0355c is 68% identity, 76% similarity, with Rv0304c is 69% identity, 82% similarity, and with Rv3347c is 70% identity and 79% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 62.2 and its translated protein sequence is as follows:

(SEQ ID NO:55)

| | |
|---|---|
| 875 | atgaagttggtcggcgtgttcgtcaacacggtgctattgcgcatt |

(SEQ ID NO.56)

| | |
|---|---|
| | M K L V G V F V N T V L L R I |
| 830 | gcggtggcccccgacctggatttcgcacacctgctcgaccaggtg |
| | A V A P D L D F A H L L D Q V |
| 785 | cgtacccgcagtctgcaagcactcgaccatcaagacatgccctat |
| | R T R S L Q A L D H Q D M P Y |
| 740 | ggcgttctggtagaccagatcaacgccgcccgctcgtcacccgct |
| | G V L V D Q I N A A R S S P A |
| 695 | ggccccttggcccaagtcatgctggcctggcaaaacaacaaaccg |
| | G P L A Q V M L A W Q N N K P |
| 650 | gccgagctggcactgggcgagctggacatcaccgaagccccggtg |
| | A E L A L G E L D I T E A P V |
| 605 | cacaccggggcggcacggatgaacttcttgttgtccctgaccgag |
| | H T G A A R M N F L L S L T E |
| 560 | cagttcaccgagagcggtgagcccgccgggatcagcggagtcgtc |
| | Q F T E S G E P A G I S G V V |
| 515 | gaataccgcaccgccatgttcactcctacggtgatcgaagccatt |
| | E Y R T A M F T P T V I E A I |
| 470 | accccattaccgaccggttggagaggctcctaa 438 |
| | T P L P T G W R G S * |

The gene interrupted in the attenuated mutant has been characterized by sequence analysis. Using the Mycobacterium database, a functional homologue of this gene has been identified in M. tuberculosis [pir E70751 emb CAA98937.1 (Z74410) (Rv0101), refNP214615.1, nrp protein].The homology with M. tuberculosis homologue Rv 0101 is 51% identity, 67% similarity.

This is a gene encoding the nrp protein. This protein has predicted function in fatty acid synthesis. That we have identified that a mutation in this gene attenuates the M. marinum strain in virulence suggests that it is required for Mycobacterium growth in the animal host.

The DNA sequence of the open reading frame interrupted in M. marinum mutant 80.1 and its translated protein sequence is as follows:

(SEQ ID NO:57)

| | |
|---|---|
| 117 | atggggggtttcaatccaggctcgtccaacacgggttcatttaac |

(SEQ ID NO:58)

| | |
|---|---|
| | M G G F N P G S S N T G S F N |
| 162 | atcgggggcgcgaacactggttggttgaattccggcagcatcaac |
| | I G G A N T G W L N S G S I N |
| 207 | accggaattctcaactcgggagacatgaacaacggcctgttcaac |
| | T G I L N S G D M N N G L F N |
| 252 | acgggagacatgaataacggcatctttttccgcggcgtcggtcag |
| | T G D M N N G I F F R G V G Q |
| 297 | ggccgcctgtacttcggaatcggactgcccgagctaacgttgccg |
| | G R L Y F G I G L P E L T L P |
| 342 | cctcttgacgttccggggatcacggttccgggcttcaatctgcct |
| | P L D V P G I T V P G F N L P |
| 387 | gccctaacactgccctcgatgtcgctacctgccattacgacgccg |
| | A L T L P S M S L P A I T T P |
| 432 | gcgaatattacggtgggtgcgtttgatctgccggggttgacgctg |
| | A N I T V G A F D L P G L T L |
| 477 ccgccgttgacgattccggcggcgacgacgccggcgaatattacg | |
| | P P L T I P A A T T P A N I T |
| 522 | gtgggtgcgtttgatctgccggggttgacgctgccgccgttgacg |
| | V G A F D L P G L T L P P L T |
| 567 | attccggcggcgacgacgccggcgaatattacggtgggagcgttt |
| | I P A A T T P A N I T V G A F |
| 612 | aaccgtttaacttgccgggcattgacgctgccgccgttgtacgat |
| | N R L T C R A L T L P P L Y D |
| 657 | tccggcggcgacgacgccggcgaatattacggtggg |
| | S G G D D A G E Y Y G G |
| | tgcgtttga 701 |
| | C V * |

The gene interrupted in the attenuated mutant has been characterized by sequence analysis. Using the Mycobacterium database, functional homologues of this gene have been identified in M. tuberculosis [pir ||E70808 emb |CAA17697.1| (AL022020) (Rv1918c) ref |NP_{—}216434.1|; pir||B70987 emb|CAB09319.1| (Z95890) (Rv1753c) ref|NP_{—}216269.1|]. The homology with the M. tuberculosis homologue Rv 1918c is 71% identity, 83% similarity and with Rv1753c is 75% identity and 83% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 27.1 and its translated protein sequence is as follows:

(SEQ ID NO:59)

| | |
|---|---|
| 581 | ctgcaaacgaacctgtattccgttcgcgacgagcacctggatgtc |

(SEQ ID NO:60)

| | |
|---|---|
| | L Q T N L Y S V R D E H L D V |
| 536 | ttcgaagaacatggcgttgaactgggtatctcggtagatttcgcc |
| | F E E H G V E L G I S V D F A |
| 491 | gaaggcgtgcggttgacggcgggaggcaagcggacagaggcggcg |
| | E G V R L T A G G K R T E A A |
| 446 | gtacgttcgaacatccgccgtctgcaggaccggggcttacccttc |
| | V R S N I R R L Q D R G L P F |
| 401 | agcatcatcacggtgttggctgggcacaccgtcagccagattcag |
| | S I I T V L A G H T V S Q I Q |
| 356 | cgcgtattcgaggagatcagccagctccagaagccggcacgactg |
| | R V F E E I S Q L Q K P A R L |
| 311 | ctcccactgttcagcggtcccgcggcgcgtccgatgaacggcgtc |
| | L P L F S G P A A R P M N G V |
| 266 | accgtcgacaagtccgacatcctcgacgcgttgatggtgttcttc |
| | T V D K S D I L D A L M V F F |
| 221 | gacctgtgggtctcggccggtatgaccccgcgggtcgatcctctt |
| | D L W V S A G M T P R V D P L |
| 176 | gatcagtatctgcgcaccgtgatcctcgagcgcatgggcttggag |
| | D Q Y L R T V I L E R M G L E |
| 131 | cgcccgggccaagaccgtgcgctgttgggtaacgacgtcctcgtc |
| | R P G Q D R A L L G N D V L V |
| 86 | atcgaccgtgacggcagactcagctgcgatgcctaccgtgagcat |
| | I D R D G R L S C D A Y R E H |
| 41 | gggggacctcggcaatattcaccgagacgaccatcgaaggc 1 |
| | G G P R Q Y S P R R P S K |

The gene interrupted in the attenuated mutant has been characterized by sequence analysis. Using the Mycobacterium database, a functional homologue of this gene has been identified in M. tuberculosis [pir ||A70772 emb |CAA97751.1| (Z73419) (Rv1285) cysD]. The homology with the M. tuberculosis homologue Rv 1285 is 22% identity, 47% similarity.

This is a gene encoding a sulfate adenylyltransferase. That we have identified that a mutation in this gene attenuates the M. marinum strain in virulence suggests that it is required for Mycobacterium growth in the animal host.

The DNA sequence of the open reading frame interrupted in M. marinum mutant 62.6 and its translated protein sequence is as follows:

(SEQ ID NO:61)

| | |
|---|---|
| 1084 | atggtgatagacctgatcaccatcgcccgagcccccgttgccggc |

(SEQ ID NO:62)

| | |
|---|---|
| | M V I D L I T I A R A P V A G |
| 1039 ttcatcccccaaacgctatcggcggaggtggctgatcacgtcgcc | |
| | F I P Q T L S A E V A D H V A |
| 994 | gcggtcgccgtcttcgggaatccgaccgacagatatcttggcggg |
| | A V A V F G N P T D R Y L G G |
| 949 | ccaataagcgagatcagcccctggtatggccataaagcgattgac |
| | P I S E I S P W Y G H K A I D |
| 904 | ttgtgtgcgcccaacgatccgatttgcacccccggcgcccttgcg |
| | L C A P N D P I C T P G A L A |
| 859 | ctgccttctcacgatgagatgttctccgcggcacacctgtcgtat |
| | L P S H D E M F S A A H L S Y |
| 814 | gcgcagtccgggatgcccagtcgggcagcgactttcgtggtgagc |
| | A Q S G M P S R A A T F V V S |
| 769 | cagctctag 761 |
| | Q L * |

The gene interrupted in the attenuated mutant has been characterized by sequence analysis. Using the Mycobacterium database, functional homologues of this gene have been identified in M. tuberculosis 1) [pir||F70756 emb|CAA98399.1| (Z74025) (Rv1984c) ref|NP_{—}216500.1|]; 2) [pir||A70565 emb|CAB08718.1| (Z95390) (Rv3452) ref|NP_{—}217969.1|]; 3) [pir|H70564 emb|CAB08717.1| (Z95390) (Rv3451) ref|NP_{—}217968.1|] . . . . The homology with the M. tuberculosis homologue for Rv1984c is 45% identity, 54% similarity; for Rv3452 is 45% identity, 57% similarity and for Rv3451 is 59% identity, 69% similarity.

This is a gene encoding a cutinase family gene (serine esterase). That we have identified that a mutation in this gene attenuates the M. marinum strain in virulence suggests that it is required for Mycobacterium growth in the animal host.

The DNA sequence of the open reading frame interrupted in M. marinum mutant 68.6 and its translated protein sequence is as follows:

| | | |
|---|---|---|
| 1176 | atgcgaagacaagcgcgaacaaaagatgttcatgcattatctatt | (SEQ ID NO:63) |
| | M R Q A R T K D V H A L S I | (SEQ ID NO:64) |
| 1131 | tcgggacgggacttactacctcgggggtggggcctgtacattcgg | |
| | S G R D L L P R G W G L Y I R | |
| 1086 | agcctcacgaaaagcttcgtacgtcagtacacgacggccatggaa | |
| | S L T K S F V R Q Y T T A M E | |
| 1041 | accaaaatcgaggtccgagacgacaccttcctcaccggagacatg | |
| | T K I E V R D D T F L T G D M | |
| 996 | acgctcggagcatttacattcacgttcgctgccggaaaacttgaa | |
| | T L G A F T F T F A A G K L E | |
| 951 | gcgggcaacggcgcggtggcccaccaccgacccgatggaatacgg | |
| | A G N G A V A H G R P D G I R | |
| 906 | atgttctcccagttcgagaatacctgcaagactctcgcattcgcg | |
| | M F S Q F E N T C K T L A F A | |
| 861 | tgggaccaccaacgccacgcgccggcaaacttcgtcgatatcaat | |
| | W D H Q R H A P A N F V D I N | |
| 816 | tttcgcggcaaccgatag 799 | |
| | F R G N R * | |

The gene interrupted in the attenuated mutant has been characterized by sequence analysis. Using the Mycobacterium database, a functional homologue of this gene has been identified in M. tuberculosis [pirE70597; embCAB08090.1 (Z94121) (Rv3884c); refNP218401.1]. The homology with the M. tuberculosis homologue Rv3884c is 29% identity, 49% similarity.

This is a gene encoding a sporulation protein. That we have identified that a mutation in this gene attenuates the M. marinum strain in virulence suggests that it is required for Mycobacterium growth in the animal host.

The DNA sequence of the open reading frame interrupted in M. marinum mutant 80.8 and its translated protein sequence is as follows:

(SEQ ID NO:65)

| | |
|---|---|
| 786 | atgaccagcaattcgggcgttctgaacattggaagcaacaatgcg |

(SEQ ID NO:66)

| | |
|---|---|
| | M T S N S G V L N I G S N N A |
| 741 | ggattcctgaactatggcaactataattccggattcagaaacacc |
| | G F L N Y G N Y N S G F R N T |
| 696 | gtctacccatcgggaacgcccgttggtaatacctctggatttgtc |
| | V Y P S G T P V G N T S G F V |
| 651 | aacgtgggagcggtcaattcgggattcttcagtaccggtaccggc |
| | N V G A V N S G F F S T G T G |
| 606 | gattag 601 |
| | D * |

The gene interrupted in the attenuated mutant has been characterized by sequence analysis. Using the Mycobacterium database, a functional homologue of this gene has been identified in M. tuberculosis [pirA70762; embCAA98335.1 (Z74020) (Rv1548c) refNP216064.1]. The homology with the M. tuberculosis homologue Rv 1548c is 52% identity and 70% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 97.4 and its translated protein sequence is as follows:

(SEQ ID NO:67)

| | |
|---|---|
| 374 | ctgcaccggcgaagtccagacattggcatcgcgaccggaagagtt |

(SEQ ID NO:68)

| | |
|---|---|
| | L G R R S P D I G I A T G R V |
| 329 | ggcggcgtatcaattcgggcgacggcgaaaggtatctggctcatg |
| | G G V S I R A T A K G I W L M |
| 284 | accgccgcagacggcccggatgaccgtgtgcagttcgaggcggaa |
| | T A A D G P D D R V Q F E A E |
| 239 | ccggttacgagaatcgcgaccatcacgctgaacaaccccacgagg |
| | P V T R I A T I T L N N P T R |
| 194 | cgcaatgcatatgacgcggcgatgcgtgatgccattgccggctac |
| | R N A Y D A A M R D A I A G Y |
| 149 | ctggaccgcgttgccgcggatgacgatctcaccgtcgtcatcttc |
| | L D R V A A D D D L T V V I L |
| 104 | cgtggcaccggcggggtctttagcgccggcgctgacatgaataac |
| | R G T G G V F S A G A D M N N |
| 59 | gcctacgggtggtacggcgaggctgacgccccggcccgcggatcc |
| | A Y G W Y G E A D A P A R G S |
| 14 | gccaacccagcggc 1 |
| | A N P A |

The gene interrupted in the attenuated mutant has been characterized by sequence analysis. Using the Mycobacterium database, a functional homologue of this gene has been identified in M. tuberculosis [pirB70693; emb CAB03655.1 (Z81331) (Rv2831) echA16 refNP 217347.1]. The homology with the M. tuberculosis homologue Rv 2831 is 43% identity and 49% similarity.

This is a gene encoding a member of the enoyl-CoA hydratase/isomerase superfamily. These proteins function in fatty acid synthesis. That we have identified that a mutation in this gene attenuates the M. marinum strain in virulence suggests that it is required for Mycobacterium growth in the animal host.

The DNA sequence of the open reading frame interrupted in M. marinum mutant 102.4 and its translated protein sequence is as follows:

(SEQ ID NO:69)

| | |
|---|---|
| 622 | atgcgggtgcttggtggtttgtatttcggtatttcgccattgaga |

(SEQ ID NO:70)

| | |
|---|---|
| | M R V L G G L Y F G I S P L R |
| 577 | gttgqggtcttgagatcggcactggaaggggacagcgtgctattc |
| | V G V L R S A L E G D S V L L |
| 532 | cctcttggtccgcccttgccacctgatgctgtggcggctaaacgg |
| | P L G P P L P P D A V A A K R |
| 487 | ggtgagtcggggctgctctgcggcttgtcggttccgctcacctgc |
| | G E S G L L C G L S V P L S W |
| 442 | ggtacggccgttccgccggatgactacgaccattgggcaccggag |
| | G T A V P P D D Y D H W A P E |
| 397 | cctgaagaaggcgccgaggccgtggtcgaagaaaacgtggatgcg |
| | P E E G A E A V V E E N V D A |
| 352 | gcagctgccggtaccgacgagtgggacgagtgggcggaatggagg |
| | A A A G T D E W D E W A E W R |
| 307 | gagtgggaggcagcaaatgccgaacctcatttcgagatgccccgt |
| | E W E A A N A E P H F E M P R |
| 262 | accagcagcgtgataccgaattcgccggcggccggttga 224 |
| | T S S V I P N S P A A G * |

The gene interrupted in the attenuated mutant has been characterized by sequence analysis. Using the Mycobacterium database, a functional homologue of this gene has been identified in M. tuberculosis [pirE70662; embCAB06164.1 (Z83860) (Rv2348c); refNP216864.1]. The homology with the M. tuberculosis homologue Rv 2348c is 73% identity and 80% similarity.

This is a gene encoding a hypothetical protein of unknown function. That we have identified that a mutation in this gene attenuates the M. marinum strain in virulence suggests that it is required for Mycobacterium growth in the animal host.

The DNA sequence of the open reading frame interrupted in M. marinum mutant 1.4 and its translated protein sequence is as follows:

(SEQ ID NO:71)

| | |
|---|---|
| 374 | atgcttgagaatgcagcggtcaatccggcgctcaacaccagacat |

(SEQ ID NO:72)

| | |
|---|---|
| | M L E N A A V N P A L N T R H |
| 419 | cgcgatgctgctcgcgagctagcgagtgcgtacctaacggacact |
| | R D A A R E L A S A Y L T D T |
| 464 | gccaagagcagtgatgacgtcgtcagtcaggccgagtttcaggcg |
| | A K S S D D V V S Q A E F Q A |
| 509 | gcgctcgacgatgtcatcgccaacgatgctgttatg |
| | A L D D V I A N D A V M |
| | aaaaagtga 553 |
| | K K * |

The gene interrupted in the attenuated mutant has been characterized by sequence analysis. Using the Mycobacterium database, a functional homologue of this gene has been identified in M. tuberculosis [pir||F70599 emb|CAB08100.1| (Z94121) (Rv3901c) ref|NP_{—}218418.1|. The homology with the M. tuberculosis homologue Rv 3901c is 65% identity and 90% similarity.

This is a gene encoding a hypothetical protein of unknown function. These proteins function in fatty acid synthesis. That we have identified that a mutation in this gene attenuates the M. marinum strain in virulence suggests that it is required for Mycobacterium growth in the animal host.

The DNA sequence of the open reading frame interrupted in M. marinum mutant 18.5 and its translated protein sequence is as follows:

(SEQ ID NO:73)

| | |
|---|---|
| 271 | atggtgacccggctgtccaacaccgatgcgtctttctatcggttg |

(SEQ ID NO:74)

| | |
|---|---|
| | M V T R L S N T D A S F Y R L |
| 226 | gagaacaccgctaccccgatgtacgtcgggtcgctgatgatcctg |
| | E N T A T P M Y V G S L M I L |
| 181 | cgccgtccgcgtgccgggttgagctatgaggcgctgctggccacg |
| | R R P R A G L S Y E A L L A T |
| 136 | gtcgancagcggttggctcagatcccgcgctaccggcagaaggtc |
| | V X Q R L A Q I P R Y R Q K V |
| 91 | cgtgaggtgcggatcggcatggcccggccggtgtggatcgacgat |
| | R E V R I G M A R P V W I D D |
| 46 | ccggacttcgacatcacctatcacgtcaggcggtcggca |
| | P D F D I T Y H V R R S A |
| | ctgccg 2 |
| | L P |

The gene interrupted in the attenuated mutant has been characterized by sequence analysis. Using the Mycobacterium database, a functional homologue of this gene has been identified in M. tuberculosis [pir||D70591 emb|CAB08335.1| (Z95121) (Rv3234c) ref|NP_{—}217751.1|]. The homology with the M. tuberculosis homologue Rv 3234c is 85% identity and 92% similarity.

This is a gene encoding a hypothetical protein of unknown function. That we have identified that a mutation in this gene attenuates the M. marinum strain in virulence suggests that it is required for Mycobacterium growth in the animal host.

The DNA sequence of the open reading frame interrupted in M. marinum mutant 76.1 and its translated protein sequence is as follows:

(SEQ ID NO:75)

| | |
|---|---|
| 680 | atgtacaccgggccgggctcgtcgccgatggtctccgccgcctcc |

(SEQ ID NO:76)

| | |
|---|---|
| | M Y T G P G S S P M V S A A S |
| 725 | gcctggaaccggttggcttccgaactctcgttcaccgccgacggc |
| | A W N R L A S E L S F T A D G |
| 770 | tacgagcgagtgatcaaggcgctatccggcgaagagtggttcgga |
| | Y E R V I K A L S G E E W F G |
| 815 | ccggcctccgcgatgatgctggaagcgatcacgccctatgtgacg |
| | P A S A M M L E A I T P Y V T |
| 860 | tggatgcgcaccaccgccgtgcaagctgaacaggcggccaagcag |
| | W M R T T A V Q A E Q A A K Q |
| 905 | gcggaagccgcggtcgccgcgtttgaggccgcgttcaccggcgtg |
| | A E A A V A A F E A A F T G V |
| 950 | gtgcccccgcccctgatcgcgtccaatcgcatgcagctgatgacc |
| | V P P P L I A S N R M Q L M T |
| 995 | ctgatggcgcggaacatctacggccagtacaccgccgagatcgca |
| | L M A R N I Y G Q Y T A E I A |
| 1040 | tncntggaagcgcagtacgccgagatgtgggcgcaggacgccagg |
| | X X E A Q Y A E M W A Q D A R |
| 1085 | gcgatgtacacctacgtcgggctcctccgcgagcgcgacaaagat |
| | A M Y T Y V G L L R E R D K D |
| 1130 | caccgcgttcacccc 1144 |
| | H R V H P |

The gene interrupted in the attenuated mutant has been characterized by sequence analysis. Using the Mycobacterium database, a functional homologue of this gene has been identified in M. tuberculosis [pir||H70503 emb|CAB10962.1| (Z98268) (Rv1705c) ref|NP_{—}216221.1 |]. The homology with the M. tuberculosis homologue Rv 1705c is 67% identity and 77% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 95.3 and its translated protein sequence is as follows:

(SEQ ID NO:77)

| | |
|---|---|
| 584 | atgttctcttcagcggcggcaacgtggggcggtacccgccaaggt |

(SEQ ID NO:78)

| | |
|---|---|
| | M F S S A A A T W G G T R Q G |
| 629 | gcatacgcggccgctaacgcttatatcgaagcactcgtaacgcgg |
| | A Y A A A N A Y I E A L V T R |
| 674 | tacgcggtcgcggttgccacgctatagccccagcgtggggggcc |
| | L R G R G C H A I A P A W G A |
| 719 | tggacagacgacagaacaacatcgcaagaagttgtgggatatttc |
| | W T D D R T T S Q E V V G Y F |
| 764 | agccgcatcgggcttcatcaaatatcccccgatatcgccttcgcc |
| | S R I G L H Q I S P D I A F A |
| 809 | gcacttcaacaatccctcgacgtagacgacaccctgattacgatc |
| | A L Q Q S L D V D D T L I T I |
| 854 | gccgatgtcgactggagtcaattccgagacgtattcaccactact |
| | A D V D W S Q F R D V F T T T |
| 899 | ggccgcgcccacaccctactggccgagctgggcaccacccaaccc |
| | G R A H T L L A E L G T T Q P |
| 944 | cagacagccgaaattcccgccatcaccgaaaactcccactacgcc |
| | Q T A E I P A I T E N S H Y A |
| 989 | gcacagctagccaagcaaaccccgcagcagcaattgacgacgctg |
| | A Q L A K Q T P Q Q Q L T T L |
| 1034 | atcgagttggtgaccactgtgactgccgcgggtattagcgcaccc |
| | E E L V T T V T A A G I S A P |
| 1079 | cgacccggcaatgttggatcccgacctgtccttcaaggacctcgg |
| | R P G N V G S R P V L Q G P R |
| 1124 | catcgactcgctgagcgcgctcgagctacgttaacacctttgact |
| | H R L A E R A R A T L T P L T |
| 1169 | cgggnacaccgggcttga 1186 |
| | R X H R A * |

The gene interrupted in the attenuated mutant has been characterized by sequence analysis. Using the Mycobacterium database, a functional homologue of this gene has been identified in M. tuberculosis [pir|A70984 emb|CAB06099.1| (Z83857) ppsC (Rv2933) ref|NP_{—}217449.1|].The homology with the M. tuberculosis homologue Rv 2933 is 34% identity and 47% similarity.

This is a gene encoding a polyketide synthetase. These proteins are involved in fatty acid synthesis. That we have identified that a mutation in this gene attenuates the M. marinum strain in virulence suggests that it is required for Mycobacterium growth in the animal host.

The DNA sequence of the open reading frame interrupted in M. marinum mutant 88.2 and its translated protein sequence is as follows:

(SEQ ID NO:79)

| | |
|---|---|
| 493 | ctgactacgggaccgggatctaggcttacacccgccaattccatt |

(SEQ ID NO:80)

| | |
|---|---|
| | L T T G P G S R L T P A N S I |
| 448 | tcgaggtgtgttttcatgaatcagccacgacaaccagcaaccacg |
| | S R C V F M N Q P R Q P A T T |
| 403 | acgggcgatgcgagcacctcaacgacgccggcgcgcaccatctgg |
| | T G D A S T S T T P A R T I W |
| 358 | cctggcatcgggcggggcttcgcccacgaggaactacccaaacac |
| | P G I G R G F A H E E L P K H |
| 313 | ctcttcaccgtcgccgctctgcacgcccgccgggcgctcgccgct |
| | L F T V A A L H A R R A L A A |
| 268 | gccgaacaccaactcgaccaactcgaccgcgccacctcgataggg |
| | A E H Q L D Q L D R A T S I G |
| 223 | acggctgtcgagctactaggcaaagccgccctcaccctcgtatcc |
| | T A V E L L G K A A L T L V S |
| 178 | cccacgctgatcgcggaaagagacgccaagagcctactgctgtac |
| | P T L I A E R D A K S L L L Y |
| 133 | tcaggtatccccgcgacatcaccgcacgaagcaaaaacaaagaca |
| | S G I P A T S P H E A K T K T |
| 88 | gcggccgaatgcctgtcaatcctgaatcactcgcattccattgac |
| | A A E C L S I L N H S H S I D |
| 43 | ttcaacctgcagagagattccaaaatatttgtcgta |
| | F N L Q R D S K I F V V |
| | cgaaac 2 |
| | R N |

The gene interrupted in the attenuated mutant has been characterized by sequence analysis. Using the protein database, this is a gene encoding a protein of unknown function. That we have identified that a mutation in this gene attenuates the M. marinum strain in virulence suggests that it is required for Mycobacterium growth in the animal host.

The DNA sequence of the open reading frame interrupted in M. marinum mutant 38.3 and its translated protein sequence is as follows:

(SEQ ID NO:81)

| | |
|---|---|
| 314 | atgagtcaggcaaccggcaatgaggtccccggcgttctggtcgcg |

(SEQ ID NO:82)

| | |
|---|---|
| | M S Q A T G N E V P G V L V A |
| 269 | ctcgactggcagacgctgacctgccagtccgacgcgggttgcaca |
| | L D W Q T L T C Q S D A G C T |
| 224 | aatcgcgcgacgcatgtcgtccacacccacgcattggaccactgc |
| | N R A T H V V H T H A L D H C |
| 179 | aaccggcccaatcttgatccgttcgggaacgtcatagacatcctg |
| | N R P N L D P F G N V I D I L |
| 134 | tgcggcgactgcctcgggcgcgcccgggccgcagcgctggtgcga |
| | C G D C L G R A R A A A L V R |
| 89 | gtgaaccgtctgggccgctcgtcgggcgcatactgcctgacctgc |
| | V N R L G R S S G A Y C L T C |
| 44 | ggggccccgctgtccgaccccggcgacatcatccgc |
| | G A P L S D P G D I I R |
| | gaacgttg 1 |
| | E R |

The gene interrupted in the attenuated mutant has been characterized by sequence analysis. Using the Mycobacterium database, a functional homologue of this gene has been identified in M. tuberculosis [ref|NP_{—}334879.1| (NC_{—}002755); gb|AAK44693.1| (AE006949)]. The homology with the M. tuberculosis homologue MT0470 is 62% identity and 76% similarity.

This is a gene encoding a hypothetical protein of unknown function. That we have identified that a mutation in this gene attenuates the M. marinum strain in virulence suggests that it is required for Mycobacterium growth in the animal host.

The DNA sequence of the open reading frame interrupted in M. marinum mutant 72.10 and its translated protein sequence is as follows:

(SEQ ID NO:83)

| | |
|---|---|
| 548 | ctggtgacacgcgcagcagggaaggatttgccgatgcccgagggc |

(SEQ ID NO:84)

| | |
|---|---|
| | L V T R A A G K D L P M P E G |
| 593 | aaccccgccaaaccactcgatgggtttcgggtgctcgatttcacc |
| | N P A K P L D G F R V L D F T |
| 638 | cagaacgttgccgggccgctggccggacaggtcctggccgacctg |
| | Q N V A G P L A G Q V L A D L |
| 683 | ggcgccgaggtgatcaaggttgaggcccccggcggtgaggcggcg |
| | G A E V I K V E A P G G E A A |
| 728 | cggcacatcaccgccgtgctgccgcaccgcccgccgctagcgacc |
| | R H I T A V L P H R P P L A T |
| 773 | tatttcctgccgaacaacaggggcaagaagtcggtgtcggtggat |
| | Y F L P N N R G K K S V S V D |
| 818 | ctgtccaccgacacggctcgccggcagatcctgcggctcgccgac |
| | L S T D T A R R Q I L R L A D |
| 863 | accgccgacgtggttgcttggaggggttttcggcccggc |
| | T A D V V A W R G F R P G |
| | gtcat 906 |
| | V |

The gene interrupted in the attenuated mutant has been characterized by sequence analysis. Using the Mycobacterium database, a functional homologue of this gene has been identified in M. tuberculosis [ref|NP_{—}217789.11 (NC_{—}000962); pir||A70979; emb|CAB07085.1| (Z92771) (Rv3272)]. The homology with the M. tuberculosis homologue Rv3272 is 84% identity and 90% similarity.

This is a gene encoding a hypothetical protein with similarity to proteins with L-carnitine dehydratase activity. That we have identified that a mutation in this gene attenuates the M. marinum strain in virulence suggests that it is required for Mycobacterium growth in the animal host.

The DNA sequence of the open reading frame interrupted in M. marinum mutant 58.14 and its translated protein sequence is as follows:

(SEQ ID NO:85)

| | |
|---|---|
| 455 | ctgaccggagatggtcagataggcatcggcggcctgaactcgggc |

(SEQ ID NO:86)

| | |
|---|---|
| | L T G D G Q I G I G G L N S G |
| 500 | tccggaaatattggtttcgggaactcgggcaacaacaatatcggc |
| | S G N I G F G N S G N N N I G |
| 545 | ttcttcaactcgggtgacaataatgtcggcttcctgaattcgggc |
| | F F N S G D N N V G F L N S G |
| 590 | agtgagaacaagggcttcatcaactcgggccttggcacgggtcga |
| | S E N K G F I N S G L G T G R |
| 635 | ggtccgaacttgagtgcgggcatcggaaattccggcgacctcaac |
| | G P N L S A G I G N S G D L N |
| 680 | acgggcctgttcaactcgggtgggtcgagcgcgactaccaacacc |
| | T G L F N S G G S S A T T N T |
| 725 | ggttggttcaactcgggctcccacaacacgggcatcggaaactcc |
| | G W F N S G S H N T G I G N S |
| 770 | ggcgacaccaatacgggtttcttcaactccggnaacctcaatacg |
| | G D T N T G F F N S G N L N T |
| 815 | ggcttgttcaactcgggtgacgtcaacacggggctc |
| | G L F N S G D V N T G L |
| | tttaattc 858 |
| | F N |

The gene interrupted in the attenuated mutant has been characterized by sequence analysis. Using the Mycobacterium database, a functional homologue of this gene has been identified in M. tuberculosis [pir|E70663 emb|CAB06165.1| (Z83860) (Rv2356c) ref|NP_{—}216872.1| (NC_{—}000962)]. The homology with the M. tuberculosis homologue Rv 2356c is 62% identity and 75% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 49.7 and its translated protein sequence is as follows:

(SEQ ID NO:87)

| | |
|---|---|
| 662 | atgctgttgtcgccatttccgcactggcgactaactggtccgtct |

(SEQ ID NO:88)

| | |
|---|---|
| | M L L S P F P H W R L T G P S |
| 617 | gtgctgcaaagaccagcggttgatgggcgggcacagacagagggc |
| | V L Q R P A V D G R A Q T E G |
| 572 | tcgtggctcggcaccgacatagtcggcagtcatcggcaatgtggt |
| | S W L G T D I V G S H R Q C G |
| 527 | ggccgaggtcctcctggccgatttctcgtggccagcgcgcgttta |
| | G R G P P G R F L V A S A R L |
| 482 | gcggattacgacgcgttccggccgacactggcgcagaacgtcatc |
| | A D Y D A F R P T L A Q N V I |
| 437 | gatttcggtggccgtacgggctacgtccgggccgcagtgcgggcc |
| | D F G G R T G Y V R A A V R A |
| 392 | ggcgtgccgattgtgccggcggtgtcgatcggcggccaggaaact |
| | G V P I V P A V S I G G Q E T |
| 347 | caactatttgtcagtcgcggcaactggctggcaaagcggttgggg |
| | Q L F V S R G N W L A K R L G |
| 302 | ctcaaacgaatccggatagagattcttcccattaccatcggctta |
| | L K R I R I E I L P I T I G L |
| 257 | ccgttcggcctgacgatgttctttcccgccaattttccgctgccg |
| | P F G L T M F F P A N F P L P |
| 212 | gcaaaatcgtctatcaggtactggagccgatcgacattgccgcca |
| | A K S S I R Y W S R S T L P P |
| 167 | gttcggcaccgaccccgacgtcgcgcaggtcgacgcccacgtgcg |
| | V R H R P R R R A G R R P R A |
| 122 | ctcggtgatgcagtcggccctcgatcggctggcgaacagcgcccg |
| | L G D A V G P R S A G E Q R P |
| 77 | atttcccgtgctgggcttgatcacccgcgggcccgaatcgccgga |
| | I S R A G L D H P R A R I A G |
| 32 | attgatggcaagcatggaaccgtgacggga 3 |
| | I D G K H G T V T G |

The gene interrupted in the attenuated mutant has been characterized by sequence analysis. Using the Mycobacterium database, a functional homologue of this gene has been identified in M. tuberculosis [pir|G70914; emb|CAB09256.1| (Z95844) (Rv1428c); ref|NP_{—}215944.1| (NC_{—}000962)|]. The homology with the M. tuberculosis homologue Rv 1428c is 70% identity and 80% similarity.

This is a gene encoding an acyltransferase family protein. These proteins function in fatty acid synthesis. That we have identified that a mutation in this gene attenuates the M. marinum strain in virulence suggests that it is required for Mycobacterium growth in the animal host.

The DNA sequence of the open reading frame interrupted in M. marinum mutant 61.5 and its translated protein sequence is as follows:

(SEQ ID NO:89)

| | |
|---|---|
| 604 | ctgttggtacacaccctccgccgccgcaatcgccggcgcattctg |

(SEQ ID NO:90)

| | |
|---|---|
| | L L V H T L R R R N R R R I L |
| 559 | accaaacaaattcgacatcaccaactgcacaaacccattgcgatt |
| | T K Q I R H H Q L H K P I A I |
| 514 | agccgccaccgccaacggatgcaccatcgccgcacgcgccgcctc |
| | S R H R Q R M H H R R T R R L |
| 469 | aaacgccgaggccaccaccttcgccgacgccgacgcccccgcggc |
| | K R R G H H L R R R R R P R G |
| 424 | ccgcgccgccgccgcccccagccaactcgcatacggccccgccgc |
| | P R R R R P Q P T R I R P R R |
| 379 | agccaccatcgccgaagccgccgcaccctgccacgcctgacccgc |
| | S H H R R S R R T L P R L T R |
| 334 | acccgcaagacccgaggtcaccgacgcaaacgactccgccgccgc |
| | T R K T R G H R R K R L R R R |
| 289 | cgccaactccgccgacaacccatcccaggccgccgccgccgccaa |
| | R Q L R R Q P I P G R R R R Q |
| 244 | catcggctcagaccccgcaccagagaacatccgcaccgaattaat |
| | H R L R P R T R E H P H R I N |
| 199 | ctccggcggcaacaccgcgaaattcatcagaatcgcccctccttc |
| | L R R Q H R E I H Q N R P S F |
| 154 | aacgggatattctcaaccgcacacccgagcgttaccgcgaccgac |
| | N G I F S T A H P S V T A T D |
| 109 | acgcaccggcacccaccggccacccaccggccagagcccggccac |
| | T H R H P P A T H R P E P G H |
| 64 | ccccgccgaacccacaacacccgagcgacattaggtcagaaccca |
| | P R R T H N T R A T L G Q N P |
| 19 | gccacaaaccccgtaatc 2 |
| | A T N P V I |

The gene interrupted in the attenuated mutant has been characterized by sequence analysis. Using the Mycobacterium database, a functional homologue of this gene has been identified in M. tuberculosis [gb|AAK44854.1| (AE006959); ref|NP_{—}335040.1| (NC_{—}002755) sensor histidine kinase]. The homology with the M. tuberculosis homologue is 31% identity and 35% similarity.

This is a gene encoding a sensor histidine kinase protein. These proteins have a regulatory function. That we have identified that a mutation in this gene attenuates the M. marinum strain in virulence suggests that it is required for Mycobacterium growth in the animal host.

The DNA sequence of the open reading frame interrupted in M. marinum mutant 114.4 and its translated protein sequence is as follows:

(SEQ ID NO:91)

| | |
|---|---|
| 838 | atggccaaacatctagcgccgcgtttcgatgacgtacaggcgcat |

(SEQ ID NO.92)

| | |
|---|---|
| | M A K H L A P R F D D V Q A H |
| 883 | tacgacctatccgacgatttcttccggctttttctggatcccacc |
| | Y D L S D D F F R L F L D P T |
| 928 | cagacctacagctgcgcctacttcgaqcgtqatgacatgacgctg |
| | Q T Y S C A Y F E R D D M T L |
| 973 | gaagaggcgcaqatcgccaagatcgacctggcgctgggcaagctg |
| | S E A Q I A K I D L A L G K L |
| 1018 | ggtttggagcccggcatgacactgctcgatatcggctgcggctgg |
| | G L E P G M T L L D I G C G W |
| 1063 | ggcgccaccatgcgccgcgcgatcgagaaatacgacgtcaacgtc |
| | G A T M R R A I E K Y D V N V |
| 1108 | gtcggcctgaccctgtccaagaaccaggccgcccacgtgcagaag |
| | V G L T L S K N Q A A H V Q K |
| 1153 | tcgttcgaccagctggacaccgcacgcacccggcgggtgctgctg |
| | S F D Q L D T A R T R R V L L |
| 1198 | gagggctgggagcagttcgatgagcccgtcgaccgcatcgtctcg |
| | S G W E Q F D E P V D R I V S |
| 1243 | atcggcgcgttcgaacacttcggtcacgaccgctacgacgacttc |
| | I G A F E R F G H D R Y D D F |
| 1288 | ttcaccctggcccacaacatcctgcccagcgacggggtgatgctg |
| | F T L A H N I L P S D G V M L |
| 1333 | ctgcacacgatcacggggctgacgatgccg 1362 |
| | L H T I T G L T M P |

The gene interrupted in the attenuated mutant has been characterized by sequence analysis. Using the Mycobacterium database, a functional homologue of this gene has been identified in M. tuberculosis [pir||A70614; emb|CAB07103.1| (Z92772) (Rv0644c); ref|NP_{—}215158.1| (NC_{—}000962) mmaA2]. The homology with the M. tuberculosis homologue Rv 0644c is 86% identity and 91% similarity.

This is a gene encoding a methoxy mycolic acid synthase. These proteins function in fatty acid synthesis. That we have identified that a mutation in this gene attenuates the M. marinum strain in virulence suggests that it is required for Mycobacterium growth in the animal host.

The DNA sequence of the open reading frame interrupted in M. marinum mutant 49.6 and its translated protein sequence is as follows:

(SEQ ID NO:93)

| | |
|---|---|
| 371 | atgcacgtacggtatcggcgatccacaccgcgagatcactcegcg |

(SEQ ID NO:94)

| | |
|---|---|
| | M R V R Y R R S T P R D H S A |
| 326 | gagtcggctgctgcagcattgtcagtgcggcaccacgaagccaca |
| | E S A A A A L S V R H H E A T |
| 281 | gtgcctgagccagcggcgccacatcgcaggcctccgccgccagca |
| | V P E P A A P H R R P P P P A |
| 236 | ccgcgaccgtgtcgccacggccaatccccatggcggccagactgt |
| | P R P C R H C Q S P W R P D C |
| 191 | ttgacatgccatgggcctgttcctgaatcgcacgccaagtcatct |
| | L T C R G P V P E S H A K S S |
| 146 | ctcggggagtatcgaccgaaccgacataaagagcgttcggggaag |
| | L G E Y R P N R H K E R S G K |
| 101 | actcggcagcggcaccaatttcacggaccaacctactactcaaga |
| | T R Q R H Q F H G P T Y Y S R |
| 56 | caaaaactcctttactcggcaaacattatcgaaacaggccgcatc |
| | Q K L L Y S A N T I E T G R I |
| 11 | ggcacataa 3 |
| | G T * |

The gene interrupted in the attenuated mutant has been characterized by sequence analysis. Using the Mycobacterium database, a functional homologue of this gene has been identified in M. tuberculosis [gi 6225691 sp P95235 (mmpL9); Rv2339]. The homology with the M. tuberculosis homologue is 26% identity and 49% similarity.

This is a gene encoding a putative membrane protein. That we have identified that a mutation in this gene attenuates the M. marinum strain in virulence suggests that it is required for Mycobacterium growth in the animal host.

The DNA sequence of the open reading frame interrupted in M. marinum mutant 91.4 and its translated protein sequence is as follows:

(SEQ ID NO:95)

| | |
|---|---|
| 262 | ctggcccgacgcgacgtatgtgacggcggtccaagatctcttcat |

(SEQ ID NO:96)

| | |
|---|---|
| | E A R R D V C D G G P R S L H |
| 307 | cgatccccatttcccggggttcagcacacaggtcctcttcacgcc |
| | R S P F P C V Q H T G P L H A |
| 352 | ggagcaactctggccatttaccagcaatctgggcagcctgacgtt |
| | C A T L A I Y Q Q S G Q P D V |
| 397 | cqgtcaatccgtcgcccagggtgtggccatattgcgagatgcgct |
| | R S I R R P G C C H I A R C A |
| 442 | cagtgcccaactcagcgacccggcaaataccgccgtcgtcttcgg |
| | Q C P T Q R P G K Y R R R L R |
| 487 | ctactcgcaaagcgccacgattgccaccaaccaaatacgcgcttt |
| | L L A K R H D C H Q P N T R F |
| 532 | catgagccagctgaatccgcccggtcccagccagttgtcgtctgt |
| | H E P A E S A R S Q P V V V C |
| 577 | gctgaccggcaatccgaacaaccccgacggcggcgtcctcgaacg |
| | A D R Q S E Q P R R R R P R T |
| 622 | cttcaacggtctttacctcccgattgtgcgattgtgqatgtgttg |
| | L Q R S L P P D C A I V D V L |
| 667 | ttcaatggcgcgaccccgccggattcgccctatcccaccgccatt |
| | F N C A T P P D S P Y P T A I |
| 712 | tacaccgcccaatacgacggcatagccaacttcccgcgctacccg |
| | Y T A Q Y D C I A N F P R Y P |
| 757 | ctcaatgtggtgtcggacgtgaacgcgataatgggcttcctttat |
| | L N V V S D V N A I M G F L Y |
| 802 | gacgagcactactacgcgggcctgacatcggacccgaacgccata |
| | D E H Y Y A C L T S D P N A I |
| 847 | gactccgggcccaccgttgccaacgcggtgcagctgccgacctcg |
| | D S C P T V A N A V Q L P T S |
| 892 | cccggctacaccggcaataccgagtactacatggtcctggcccag |
| | P C Y T C N T E Y Y M V L A Q |
| 937 | catctaccgcttaccgacccgcttcgtcagatcccgtacgtggga |
| | H L P L T D P L R Q I P Y V C |
| 982 | acacccatcgccgatctgatccagccctcgctgcgggtgatcgtg |
| | T P T A D L I Q P S L R V I V |
| 1027 | gacttgggctacagcgattacggaccgggacagaactatgcggac |
| | D L C Y S D Y C P C Q N Y A D |
| 1072 | atccccacccccgcctcactgttctcgctgcccaacccgctcgcg |
| | I P T P A S L F S L P N P L A |
| 1117 | gtgagctattacctgggcaagggtgccgtgcagggggtgcaggca |
| | V S Y Y L C K G A V Q G V Q A |
| 1162 | ttcatggtggatgagggatqgttgccgcagtcctacctgcccgac |
| | F M V D E C W L P Q S Y L P D |
| 1207 | acctacccctacgtggcgtcgttgtcgcccgggctgaacgtttat |
| | T Y P Y V A S L S P C L N V Y |
| 1252 | ctgggccanccaaacgtgaccggactatcgctgctgaccggcgcc |
| | L C Q P S V T C L S L L T G A |
| 1297 | ctgggaaccgggggtttcgcgacctgggatgga 1329 |
| | L C T C C F A T W D C |

The gene interrupted in the attenuated mutant has been characterized by sequence analysis. Using the Mycobacterium database, a functional homologue of this gene has been identified in M. tuberculosis [pir||F70839; emb|CAA17315.1| (AL021927) (Rv0160c); ref|NP_{—}214674.1| (NC_{—}000962)]. The homology with the M. tuberculosis homologue Rv 0160c is 59% identity and 72% similarity.

This is a gene encoding a PE protein of unknown function. That we have identified that a mutation in this gene attenuates the M. marinum strain in virulence suggests that it is required for Mycobacterium growth in the animal host.

The DNA sequence of the open reading frame interrupted in M. marinum mutant 135.11 and its translated protein sequence is as follows:

(SEQ ID NO:97)

| | |
|---|---|
| 12 | atggtgtgtcgggggttcggtaacttcggtgccacggtgtcgggt |

(SEQ ID NO:98)

| | |
|---|---|
| | M V C R G E C N F G A T V S C |
| 57 | tggggcaacgtcgcgtcgcatgcgtcgggttttgagaactttggc |
| | W G N V A S H A S G F E N F G |
| 102 | accgggttgtcggggttcaccaatatgggtgatgtgttgtcgggg |
| | T C L S C F T N M G D V E S G |
| 147 | ttgaagaacaccaacagttcgggtctggggacctcgggtgtgggc |
| | L K N T N S S C L C T S G V C |
| 192 | aacgtgggtgacagtctgtcggggttgttctacgcgggtccggac |
| | N V G D S L S C L F Y A C P D |
| 237 | cggatgagcatttttaatgctgggttggggaatttgggtgtgggg |
| | R M S I F N A C L C N L C V C |
| 282 | aatgttgggtttgcgagtgtgggtgatgggaatgttggtgggggt |
| | N V C F A S V C D C N V C C C |
| 327 | aatctcggtgatgggaatgttgggtttgggaatgttggtggcctg |
| | N L C D C N V C F C N V C C L |
| 372 | aactttggttctgggaactggggtggtttcaacctgggttcgggg |
| | N F C S C N W C C F N E C S C |
| 417 | aatattggttcgtataatttcgggccggggaacntgggttcqtac |
| | N T C S Y N F C P C N X C S Y |
| 462 | aatattgggtttggtaatgcgggtgactataacgttggtttcggt |
| | N I C F C N A C D Y N V C F C |
| 507 | aatagtgggttggggaatatcgggtttgggaatagtgggagcaat |
| | N S C E C N I C F C N S C S N |
| 552 | aatctggggatcgggctgaccggtagtggtcaggtggggtttggg |
| | N E C I C E T C S C Q V C F C |
| 597 | ggtttgggggctggaactccgggagtgggaatgtggggttgttca |
| | C L C A C T P C V C M W C C S |
| 642 | actccggggatgggaatgtggggttgttcaactccgggaccggta |
| | T P C M C M W C C S T P C P V |
| 687 | actggggtgtgggtaactcgggtgagtttgatacggggttgttca |
| | T C V W V T R V S E I R C C S |
| 732 | acgcggggcgctacaacaccgggqtgttcaactcgg |
| | T R C A T T P C C S T R |
| | gtgtgttga 776 |
| | V C * |

The gene interrupted in the attenuated mutant has been characterized by sequence analysis. Using the Mycobacterium database, a functional homologue of this gene has been identified in M. tuberculosis [pir||D70575 emb|CAB08587.1| (Z95324) (Rv0355c) ref|NP_{—}214869.1| (NC_{—}000962)]. The homology with the M. tuberculosis homologue Rv 0355c is 48% identity and 65% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

The DNA sequence of the open reading frame interrupted in M. marinum mutant 39.14 and its translated protein sequence is as follows:

| | | |
|---|---|---|
| 686 | atgtcggcgatgctcgggcgcaacagctaccgggccaagtcggtt | (SEQ ID NO:99) |
| | M S A M L G R N S Y R A K S V | (SEQ ID NO:100) |
| 731 | gacgcggtqgtcgacgagatcgcgtacctgaaatccgatttcgac | |
| | D A V V D E I A Y L K S D F D | |
| 776 | attggctttctctgcatcaccgacgacctgttcatctccaagcat | |
| | I G F L C I T D D L F I S K H | |
| 821 | cccagctcgcaacaacgcgcggccgagtttgctgacgccatgatc | |
| | P S S Q Q R A A E F A D A M I | |
| 866 | aacagcggcgttgacgtcaagttcatgatggatatccgcctggac | |
| | N S G V D V K F M M D I R L D | |
| 911 | tccgtggtggatctagaactgttcaaacacctgcacaaagcgggt | |
| | S V V D L E L F K H L H K A G | |
| 956 | ttgcgccgggttttcgtcggcttggaaaccggttcttatgatcaa | |
| | L R R V F V G L E T G S Y D Q | |
| 1001 | ctccgcgcgtaccggcaaacagatcatcaatcgcggacaagatqc | |
| | L R A Y R Q T D R Q S R T R C | |
| 1046 | cgccgacacgatcaacgcactgcagcaggtggg | |
| | R R H D Q R T A A G G | |
| | cgttga 1084 | |
| | R * | |

The gene interrupted in the attenuated mutant has been characterized by sequence analysis. Using the Mycobacterium database, a functional homologue of this gene has been identified in M. tuberculosis [pir||C70960 emb|CAB07008.1| (Z92669) (Rv0213c) ref|NP_{—}214727.1| (NC_{—}000962)]. The homology with the M. tuberculosis homologue Rv 0213c is 77% identity and 91% similarity.

This is a gene encoding a putative methyl transferase. That we have identified that a mutation in this gene attenuates the M. marinum strain in virulence suggests that it is required for Mycobacterium growth in the animal host.

The DNA sequence of the open reading frame interrupted in M. marinum mutant 95.18 and its translated protein sequence is as follows:

| | | |
|---|---|---|
| 12 | atggcgtccattcgctgcggcaagtcgatccggttccgctctagg | (SEQ ID NO:101) |
| | M A S T R C G K S I R F R S R | (SEQ ID NO:102) |
| 57 | gacaccacgtttcccgaacggttcgagaagaatcagccggtcgtc | |
| | D T T F P E R F E K N Q P V V | |
| 102 | atcgcggcactgatcacgcttcccgtagcaattctctttgtctac | |
| | I A A L I T L P V A I L F V Y | |
| 147 | gacgcacagcacgccttctatagaaatttctacttgaaccacttg | |
| | D A Q H A F Y R N F Y L N H L | |
| 192 | acgtcggtcgccgcttgcctgattctcgcgtcggtctccggtccc | |
| | T S V A A C L I L A S V S G P | |
| 237 | ctcgctctccgcttcaccaagggcgctagcgtcctcgttggaatc | |
| | L A L R F T K G A S V L V G I | |
| 282 | gtggtcggggcctcgttgatattcaacgcattcctgttcgtacgg | |
| | V V G A S L I F N A F L F V R | |
| 327 | ccgttggcgaacgggtatgagggcccgtcgctatccgtcctgcgc | |
| | P L A N G Y E C P S L S V L R | |
| 372 | aggtggccgcaagtatcgcgcgacaccgccgaactggcgcgggtg | |
| | R W P Q V S R D T A E L A R V | |
| 417 | tgcaaaatggacctagggcggggccgtatcatcgtcgacgatctc | |
| | C K M D L G R G R I T V D D L | |
| 462 | acccaggcgggcgtttactcgtttactccaggc | |
| | T Q A G V Y S F T P G | |
| | caatga 500 | |
| | Q * | |

The gene interrupted in the attenuated mutant has been characterized by sequence analysis. Using the Mycobacterium database, a functional homologue of this gene has been identified in M. tuberculosis [gb|AAK44468.1| (AE006933); ref|NP_{—}334654.1| (NC_{—}002755)]. The homology with the M. tuberculosis homologue is 38% identity and 50% similarity.

This is a gene encoding a hypothetical protein of unknown function. That we have identified that a mutation in this gene attenuates the M. marinum strain in virulence suggests that it is required for Mycobacterium growth in the animal host.

The DNA sequence, of the open reading frame interrupted in M. marinum mutant 125.20 and its translated protein sequence is as follows:

| | | |
|---|---|---|
| 451 | atgcgccgcctcgcgagtagttttcgagttttcggagaccgttat | (SEQ ID NO:103) |
| | M R R L A S S F R V F G D R Y | (SEQ ID NO:104) |
| 496 | caaaacggcgccgttatgggagatggcgcggcagccgtcgtcctt | |
| | Q N G A V M G D G A A A V V L | |
| 541 | tcgaagaaagaagggtttgcccggctgatcgctagtaatagaact | |
| | S K K E G F A R L I A S N R T | |
| 586 | tcattcgcggacttcgaatttctgatgcgaaatacgggatcagtg | |
| | S F A D F E F L M R N T G S V | |
| 631 | aaaaatttcgaaatgaaatttgccctagaacagatcggatacggt | |
| | K N F E M K F A L E Q I G Y G | |
| 676 | ccatatgtcgggacactgtctcgtattgttaaagaggcgatcagt | |
| | P Y V G T L S R I V K E A I S | |
| 721 | gcaaccttggaagatgcaaaaatctcagttgatgacgtttcacac | |
| | A T L E D A K I S V D D V S H | |
| 766 | ttctgcccaccagcggtctaccgactttcgcttgaggaaacattt | |
| | F C P P A V Y R L S L E E T F | |
| 811 | attggtgccagcggtataccgtttgagaagacatgc | |
| | I G A S G I P F E K T C | |
| | tggtca 852 | |
| | W S | |

The gene interrupted in the attenuated mutant has been characterized by sequence analysis. Using the protein database the most significant homologue is with Streptomyces beta-keto acyl synthase III, dbj|BAB69224.1| (AB070943). The homology is 28%.

This is a gene encoding a putative beta-keto acyl synthase III. That we have identified that a mutation in this gene attenuates the M. marinum strain in virulence suggests that it is required for Mycobacterium growth in the animal host.

The DNA sequence of the open reading frame interrupted in M. marinum mutant 62.20 and its translated protein sequence is as follows:

| | | |
|---|---|---|
| 746 | ctggggacaatccggagacgctggaggcgtttctcaaacggcacc | (SEQ ID NO:105) |
| | L G T I R R R W R R F S N G T | (SEQ ID NO:106) |
| 701 | gcttctcgtcgttcttcgtcgactacttcatcacgccgttggtgg | |
| | A S R R S S S T T S S R R W W | |
| 656 | ccgccgtgtggtcgtgcgccgccggcgatgcgctgcgctacccgg | |
| | P P C G R A P P A M R C A T R | |
| 611 | cccggtatctgttcgtctttctcgagcatcacgrcatgctgtcgg | |
| | P G I C S S F S S I T X C C R | |
| 566 | ttttcgccgcttcttctccggcacaccccagcccaaccagtaccc | |
| | F S P L L E R H T P A Q P V P | |
| 521 | ctcgatcacagcgcqaaagcagctgatcgtctctacaccgtcaag | |
| | L D H S A K A A D R L Y T V K | |
| 476 | tgcgaagagccggttttcggatcaccgacgtggcgcactgtcacc | |
| | C E E P V F G S P T W R T V T | |
| 431 | ggtggcagcgtcaactatgtgcgcgccatcgcctcgagtctggac | |
| | G G S V N Y V R A I A S S E D | |
| 386 | gaggttcgcaccggcgccgcggtgcattcgctgcgccggacggcc | |
| | E V R T G A A V H S E R R T A | |
| 341 | gacggggtcgtgatacgggccggtggcgacgcgccccgctgtttc | |
| | D G V V I R A G G D A P R C F | |
| 296 | gatgccgctgtcgtcgccgtccaccccgatcaagccctgctgttg | |
| | D A A V V A V H P D Q A E E E | |
| 251 | ctcgatgatccgacgacctgggagcgcaacgtcttgggggcaatc | |
| | E D D P T T W E R N V E G A I | |
| 206 | ccctactcgaccaatcgcgccctgctgcacaccgacgaatcggtg | |
| | P Y S T N R A E E H T D E S V | |
| 161 | ctgycacggcaccaccgagcccgggcatcgyggaactacctggtg | |
| | E X R H H R A R A S X N Y E V | |
| 116 | gcccnccggacaggaccatgtggtggtcaagctacgacgttcagc | |
| | A X R T G P C G O Q A T T F S | |
| 71 | caggttgatgcgcatcggcggcaacccgccgtttcgtggtcaccc | |
| | Q V D A H R R Q P A V S W S P | |
| 26 | tcggtggccaacgaccgggttggg 3 | |
| | S V A N D R V G | |

The gene interrupted in the attenuated mutant has been characterized by sequence analysis. Using the Mycobacterium database, a functional homologue of this gene has been identified in M. tuberculosis [pir||B70831; emb|CAA17406.1| (AL021932) (Rv0449c); ref|NP_{—}214963.1| (NC_{—}000962)]. The homology with the M. tuberculosis homologue is 76% identity and 80% similarity.

This is a gene encoding a probable dehydrogenase. That we have identified that a mutation in this gene attenuates the M. marinum strain in virulence suggests that it is required for Mycobacterium growth in the animal host.

The DNA sequence of the open reading frame interrupted in M. marinum mutant 68.12 and its translated protein sequence is as follows:

| | | |
|---|---|---|
| 134 | atgctccgcgttacccgcaacggagcggtccaggcccgcaccgcc | (SEQ ID NO:107) |
| | M L R V T R N G A V Q A R T A | (SEQ ID NO:108) |
| 179 | accctcaacaccctgcgctcgctcgtcatcaccgctcccgagccg | |
| | T L N T E R S L V I T A P E P | |
| 224 | ctacgcacccagctgcgttccctaacctccgcgcagcttgttacc | |
| | L R T Q L R S E T S A Q L V T | |
| 269 | gcttgcgcacacctgcgtcccgacctgaccaaactcgccgacccc | |
| | A C A H E R P D E T K L A D P | |
| 314 | gtccaggcagctaaacacgcottacgttcgatggctcLgcgcgct | |
| | V Q A A K H A E R S M A E R A | |
| 359 | caacacctcaataccgaaacacggactctgcgaatgcaactcaat | |
| | Q H E N T E T R T E R M Q E N | |
| 404 | gacctgacccaagctgcagcacccgccactagtgccgtattcggg | |
| | D L T Q A A A P A T S A V F C | |
| 449 | ctcggtccagacaccgtctccgcactgctgatcaccattggcgat | |
| | E G P D T V S A E L I T I C D | |
| 494 | aacccagaccggctacgcagcgaagccgccttcgcccacctctgc | |
| | N P D R E R S E A A F A H E C | |
| 539 | ggggtcgcccccatccctgcatcctcgggcaaaaccgcaaaaccc | |
| | C V A P I P A S S G K T A K P | |
| 584 | accgacaccgactgcaccgcggcggcgaccgggccgccaacagcg | |
| | T D T D C T A A A T G P P T A | |
| 629 | ccctacacatcgccacagtcgtccggctgcgctacgacccccgca | |
| | P Y T S P Q S S G C A T T P A | |
| 674 | gccgcgcctacgccgaccgccgcaccaccgagggcctgtccatgc | |
| | A A P T P T A A P P R A C P C | |
| 719 | ccgaaatcattcgctgccagaagcgctacctggcccgcgaaatct | |
| | P K S F A A R S A T W P A K S | |
| 764 | tcgacgcactacgcgccgactacgcccaactcagcacttgacatc | |
| | S T H Y A P T T P N S A L D I | |
| 809 | tataggagcgtccttcgcgatctcccgcagcgogtccttctccga | |
| | Y R S V E R D E P Q R V E E R | |
| 854 | ctcagcatcagccagcaaccgctttaa 880 | |
| | E S I S Q Q P L * | |

The gene interrupted in the attenuated mutant has been characterized by sequence analysis. Using the Mycobacterium database, a functional homologue of this gene has been identified in M. tuberculosis [ref|NP_{—}215312.1| (NC_{—}000962); pir||D70520; emb|CAB09573.1| (Z96797) (Rv0797)]. The homology with the M. tuberculosis homologue is 29% identity and 40% similarity.

This is a gene encoding a transposase. That we have identified that a mutation in this gene attenuates the M. marinum strain in virulence suggests that it is required for Mycobacterium growth in the animal host.

The DNA sequence of the open reading frame interrupted in M. marinum mutant 129.8 and its translated protein sequence is as follows:

| | | |
|---|---|---|
| 630 | atgcgtggacatccggacatcctqcaatgggtgcctctgcttggc | (SEQ ID NO:109) |
| | M R G H P D I L Q W V P L L G | (SEQ ID NO:110) |
| 585 | gctgcaggcatcgggtcggtgatcaccagctatgtcggagcaggt | |
| | A A G I G S V T T S Y V G A G | |
| 540 | aaggctaggcgcgaggtgcgcagcgctgttctagaagctctggct | |
| | K A R R E V R S A V L E A L A | |
| 495 | atgactgagggttctcggtgggcaggtctggacaaggaccacccc | |
| | M T E G S R W A G L D K D H P | |
| 450 | acattcaaaaccgcgagccgcgactttgaaaccgctgctctcatt | |
| | T F K T A S R D F E T A A L I | |
| 405 | gctcggatacccaggcccgctgtgcagcaatacctcgttctagcg | |
| | A R I P R P A V Q Q Y L V L A | |
| 360 | gacgccgcccgccggtacagcgtggaggactatgccataaagggc | |
| | D A A R R Y S V E D Y A I K G | |
| 315 | tgcgacgaggagattggcgccggggcgattaactcggacttgggc | |
| | C D E E I G A G A I N S D L G | |
| 270 | aatgttgtccaagagtcggctgagattgtcacccagctcgcatgg | |
| | N V V Q E S A E I V T Q L A W | |
| 225 | cgcccatggtggtcacgggttacatatcgcgtcaagctgagaaaa | |
| | R P W W S R V T Y R V K L R K | |
| 180 | gtacgcaataaggcaacagacatcgacaataaagacgtcaggcaa | |
| | V R N K A T D I D N K D V R Q | |
| 135 | cagctcgtctacgcgcagtgggcgctgacaaggtcccccggttca | |
| | Q L V Y A Q W A L T R S P G S | |
| 90 | ctcggtgagctgtacgacgaatacttctcggacaggaag | |
| | L G E L Y D E Y F S D R K | |
| | aagaagtag 43 | |
| | K K * | |

The gene interrupted in the attenuated mutant has been characterized by sequence analysis. Using the Mycobacterium database, a functional homologue of this gene has been identified in M. leprae [ref|NP_{—}301540.1| (NC_{—}002677); pir||T45314 probable ferredoxin; emb|CAB11006.1| (Z98271)]. The homology with the Mycobacterium homologue is 28% identity and 43% similarity. A homologue is also found in M. tuberculosis (Rv 3106).

This is a gene encoding a putative NADPH-ferredoxin reductase. That we have identified that a mutation in this gene attenuates the M. marinum strain in virulence suggests that it is required for Mycobacterium growth in the animal host.

The DNA sequence of the open reading frame interrupted in M. marinum mutant 60.2 and its translated protein sequence is as follows:

| | | |
|---|---|---|
| 1133 | atgaaatcggagcgattgtcgcgccnggagatcgcgatcaacccc | (SEQ ID No:111) |
| | M K S E R L S R X E I A I N P | (SEQ ID NO:112) |
| 1088 | attaatttcaacaagttcatgatcggtaacgagggattcgccact | |
| | I N F N K F M I G N E G F A T | |
| 1043 | gaccgtccacatccggtggtggagccggcccggtcaccatcccgg | |
| | D R P H P V V E P A R S P S R | |
| 998 | tttttcatttgtcgccgactgccggggtttgggaattccggtggt | |
| | F F I C R R L P G F G N S G G | |
| 953 | gcgccgtcgtcgggtttctttaattctggggatggtgtgtcgggg | |
| | A P S S G F F N S G D G V S G | |
| 908 | ttcggtaacttcggtgccacggtgtcgggttggggcaacgtcgcg | |
| | F G N F G A T V S G W G N V A | |
| 863 | tcgcatgcgtcgggttttgagaactttggcaccgggttgtcgggg | |
| | S H A S G F E N F G T G L S G | |
| 818 | ttcaccaatgtgggtgatgtgttgtcggggttgaagaacaccaac | |
| | F T N V G D V L S G L K N T N | |
| 773 | agttcgggtctggggacctcgggtgtgggcaacgtgggtgacagt | |
| | S S G L G T S G V G N V G D S | |
| 728 | ctgtcggggttgttctacgcgggtccggaccggatgagcattttt | |
| | L S G L F Y A G P D R M S I F | |
| 683 | aatgctgggttggggaatttgggtgtggggaatgttgggtttgcg | |
| | N A G L G N L G V G N V G F A | |
| 638 | agtgtgggtgatgggaatgttggtgggggtaacctcggtgatggg | |
| | S V G D G N V G G G N L G D G | |
| 593 | aatgttgggtttgggcttgttggtggcctggaccctttggttctg | |
| | N V G F G L V G G L D P L V L | |
| 548 | ggaactggggtggtttcaacctgggttcggggaatattggttcgt | |
| | G T G V V S T W V R G I L V R | |
| 503 | ataatttcgggccggggaacttgggttcgtacaatattggttttg | |
| | I I S G R G T W V R T I L V L | |
| 458 | gtaatgcgggtgactataacgttggtttcggtaatagtgggttgg | |
| | V M R V T I T L V S V I V G W | |
| 413 | ggaatatcgggtttgggaatagtgggagcaataatctggggatcg | |
| | G I S G L G I V G A I I W G S | |
| 368 | ggctga 363 | |
| | G * | |

The mutant (60.2), when tested individually in the goldfish model, exhibits attenuated virulence (reduced Competitive Index. See, e.g., FIG. 12) as compared to the wild type organism.

The gene interrupted in the attenuated mutant has been characterized by sequence analysis. Using the Mycobacterium database, functional homologues of this gene have been identified in M. tuberculosis [pir||B700969 emb|CAA|5732.1| (AL009198) (Rv3347c) ref|NP_{—}217864.1| (NC_{—}000962)]. The homology with the M. tuberculosis homologue Rv 3347c is 57% identity and 65% similarity.

This is a gene encoding a member of the PPE family of proteins. These proteins have no known function. That we have identified that a mutation in this gene attenuates the M. marinum strain in virulence suggests that it is required for Mycobacterium growth in the animal host.

Based on the sequence analysis to the Mycobacterium database, the gene identified as interrupted from mutant 60.2 has a functional homologues in M. tuberculosis [pir||B700969 emb|CAA15732.1| (AL009198) (Rv3347c) ref|NP_{—}217864.1| (NC_{—}000962)]. This is a gene encoding a PPE protein. This gene is a virulence gene in M. marinum and M. tuberculosis.

The DNA sequence of the open reading frame interrupted in M. marinum mutant 67.1 and its translated protein sequence is as follows:

| | | |
|---|---|---|
| 629 | atggagcataccgatagtcttcgacctttccgcctttccgcggca | (SEQ ID NO:113) |
| | M E H T D S L R P P R L S A A | (SEQ ID NO:114) |
| 674 | gacatcgacagctatggcctcaaagaaggtggaagcgcggttctc | |
| | D I D S Y G L K E G G S A V L | |
| 719 | gagtacctcggcgcccccatggcaattttcgacatcaccgagata | |
| | E Y L G A P M A I F D I T E I | |
| 764 | tacgaatacgacctcgatcagatggccgaaaagacctacggcacc | |
| | Y E Y D L D Q M A E K T Y G T | |
| 809 | acggatctgagacatcccggcgtcaagaagacgaaagcgtataag | |
| | T D L R H P G V K K T K A Y K | |
| 854 | gatcggttcatcgggggcggaatcacgctaatcaacgaaccggtt | |
| | D R F I G G G I T L I N E P V | |
| 899 | ttcaacgcgccattcagcaacttctggctgaccccacggcagcat | |
| | P N A P P S N F K L T P R Q H | |
| 944 | cgcgacgcgttgcgaaagaagggctggaagaatgtcgtcgcgcat | |
| | R D A L R K K G W K N V V A H | |
| 989 | cagaccaggaatgtcccacacacgggccacgaagccctgatgaag | |
| | Q T R N V P H T G H E A L M K | |
| 1034 | caagcctggtttgccgccaacgaggaccagtccgtcgacacgcta | |
| | Q A K P A A N E D Q S V D T L | |
| 1079 | aagaccggcatcctggtcaacgccatcatcggacaaaagagggtt | |
| | K T G I L V N A I I G Q K R V | |
| 1124 | ggcgactacatcgacgaagcgatcctgctgacgcaagatgcgttg | |
| | G D Y I D E A I L L T Q D A L | |
| 1169 | cggaccaatggatactttcgcgaaaacgtgcacatgggtgtcctt | |
| | R T N G Y P R E N V H M G V L | |
| 1214 | cacgctctgggacatgcgctatgccggcccccgagaggccatctt | |
| | H A L G H A L G R P P R G H L | |
| 1259 | ccacgcgattctcaggacgaatctcgggtgcacacatcacat 1300 | |
| | P R D S Q D S S R V H T S H | |

The gene interrupted in the attenuated mutant has been characterized by sequence analysis.

This is a gene encoding a sulfate adenylyltransferase. The homology noted to the sulfate adenylyltransferase enzymes suggests that mutant 67.1 is attenuated in its ability to respond to sulfate starvation as this enzyme is required for growth in defined synthetic medium with sulfate as a sulfur source. This suggests that in the animal host a sulfur source is limiting and thus interruption of this gene attenuates growth of the organism in the animal host.

Based on the sequence analysis to the entire database, the gene identified as interrupted from mutant 67.1 is a sulfate adenylyltransferase with homology to diverse organisms including Pyrococcus abyssi, Synechocystis sp., and Bacillus subtilis. The sequence identity is 59%, similarity 73%. No homology was found to an M. tuberculosis gene.

Gene 41.2

The full-length DNA sequence of the open reading frame disrupted in mutant 41.2, and the translation product thereof, are as follows:

| | | |
|---|---|---|
| | atgggtgatggcaaaagggacgccactcctggccatcggcgcgcc | (SEQ ID NO:115) |
| | M G D C K R D A T P G H R R A | (SEQ ID NO:116) |
| 46 | tccggcacccaccggggtgatgaattgattaccccaaaccgagag | |
| | S G T H R G D E L I T P N R E | |
| 91 | gaaatgcgaggcgccgcgccgtgggagcggttctcggccgcacct | |
| | E M R C A A P W E R F S A A P | |
| 136 | gtcgatgacgacctcgttcgatggtcgagtgcacgatccgccgac | |
| | V D D D L V R K S S A R S A D | |
| 181 | ctggcgcaggccgccgcggcggtcgacacccggggtggcgcgcaa | |
| | L A Q A A A A V D T R G C A Q | |
| 226 | ccgcgccaagcccacgacgacgtcgagcgcaccccgaaggttggc | |
| | P R Q A H D D V E R I P K V C | |
| 271 | tctcacatcgacggcggtgtcagcgtcgccgagttgatcgccaaa | |
| | S H I D C G V S V A E L I A K | |
| 316 | ctcggggcccccgttcccgcccatccggcccaccaccacagcgca | |
| | L G A P V P A H P A H H H S A | |
| 361 | ccggaatccgggcccgacccaacccccgcggatgccgcccccgac | |
| | P E S C P U P T P A D A A P D | |
| 406 | atcgcggaccaggtccacgagcctgacgagcagctggacaccgag | |
| | I A U Q V H E P D E Q L D T E | |
| 451 | gtcatcgctatcccggcctactcgctgcaactgctctccgaactc | |
| | V I A I P A Y S L Q L L S E L | |
| 496 | cccgacctcgggtctgccaactatccgcacgacgagtccgacccc | |
| | P D L G S A N Y P H D E S D P | |
| 541 | gaatcgcccggcgagcagccagccgcaccggcgcgggcccggcgg | |
| | E S P G E Q P A A P A R A R R | |
| 586 | ccgcggttgcgtcgcaggtcgaccgcaaaggctccccggcccggt | |
| | P R L R E R S T A K A P R P C | |
| 631 | aaggacgcgccgaaatcgcgccggcgcccgatactgctggccggg | |
| | K D A P K S R R E P I L L A G | |
| 676 | cggtcgctggcggcgctgttcgccgtgctggcgctggtgctcacc | |
| | R S L A A L F A V L A L V L T | |
| 721 | ggcggggcgtgggaatggagttcgtcgaaaaacaaccggctcaac | |
| | C C A W E K S S S K N N R L N | |
| 766 | acggtgagcgcgctcgacccgcactcgggcgacatcgtcaacccc | |
| | T V S A L D P H S C D I V N P | |
| 811 | agcgggcaatacggcgacgagaatttcctgatcgtcggcatggat | |
| | S C Q Y C D E N F L I V C M D | |
| 856 | acccgtgccggcgccaattccaatgtgggcgccggtgacaccgag | |
| | T E A C A N S N V G A G D T E | |
| 901 | gacgccggcggggcgcggtcggacaccgtgatgctggtcaacatc | |
| | D A C C A E S D T V M L V N I | |
| 946 | ccggcgaaccgcaagcgagtggtggcggtctcgttcccccgcgac | |
| | P A N E K R V V A V S F P R D | |
| 991 | ctggcgatcacccccgtcaagtgcgaggcctggaaccccgacacc | |
| | L A I T P V K C E A K N P D T | |
| 1036 | ggcaagtacgggccgatctatgacgagacgacgggacagatgggt | |
| | G K Y C P I Y D E T T C Q M C | |
| 1081 | ccccggatggtctacaccgagaccaaactgaactcgtcgttctcc | |
| | P R M V Y T E I K L N S S F S | |
| 1126 | ttcggcgggcccaagtgtctggtgaaggtgatccaaaaactgtcc | |
| | F C C P K C L V K V I Q K L S | |
| 1171 | gggttgagcatcaaccggttcatcgccatcgacttcgtcggcttc | |
| | C L S I N E F I A I D F V C F | |
| 1216 | gccaagatggtccaagcgctcggtggtgtcgaggtgtgcagcacc | |
| | A K M V Q A L C C V S V C S T | |
| 1261 | acgccgctgcgcgactacgaaatcggcacggtgctcgaacacgct | |
| | T P L R D Y E I C T V L E H A | |
| 1306 | gggcgccaggtgatcgacgggacgaccgccctgaactatgtgcga | |
| | C R Q V I D C T T A L N Y V R | |
| 1351 | gcccgccaggtgaccaccgagagcaacggcgactacggccgcatc | |
| | A R Q V T T E S N C D Y C R I | |
| 1396 | aaacgtcagcagctgttcttgtcgtcgttgctgcgttcgctgatt | |
| | K R Q Q L F L S S L L R S L I | |
| 1441 | tccgaagacaccctgttcaacctcaacaagctcaacaacgtggtc | |
| | S E D T L F N L N K L N N V V | |
| 1486 | gacatgttcateggcgacagctacgtcgacaacgtcaagaccaag | |
| | D M F I G D S Y V D N V K T K | |
| 1531 | gatctggttgagctgggtcagtcgctgcagggcatggcagccgga | |
| | D L V E L G Q S L Q G M A A G | |
| 1576 | cacatcacgttcgtcaccgtgcccaccggtatcaccgatgagaac | |
| | H I T F V T V P T C I T D E N | |
| 1621 | ggcgacgagcccccgcgaacggccgacatgaaggcgctgttcagc | |
| | G D E P P R T A D M K A L F S | |
| 1666 | gccatcatcgacgatgagccgctgccgctggaaaacgatcacaac | |
| | A I I D D E P L P L E N D H N | |
| 1711 | gcccagacgttgggaaaccggccgaccacgacggcaccgaccacg | |
| | A Q T L C N R P T T T A P T T | |
| 1756 | gcccccaaagcgccgccggcaagtcctgccgacgaggttcagcgc | |
| | A P K A P P A S P A D E V Q R | |
| 1801 | caacaggtgacaaccacctcgccgcaagaagtcaccgtgcaggtc | |
| | Q Q V T T T S P Q E V T V Q V | |
| 1846 | tccaacggaaccgggaccacgggtctggccgccgccgccgccagc | |
| | S N C T G T T C L A A A A A S | |
| 1891 | cagctcgagcgcaacggcttcaacgtgatggcacccgacgactac | |
| | Q L E R N C P N V M A P D D Y | |
| 1936 | ccgaattcgttgcagaccacgacggtgctttttgcccccggcaac | |
| | P N S L Q T T T V L P A P C N | |
| 1981 | gagcaagccgccgcgacggtggccgccgcgttcggcaacagcaag | |
| | E Q A A A T V A A A F C N S K | |
| 2026 | gttgagcgggtcaccgggatcggcgaggtggtgcaggtggtgctc | |
| | V E R V T C I G E V V Q V V L | |
| 2071 | ggcgccgacttcaaggcggtgaccgctcccccgccgagcggctcg | |
| | G A D F K A V T A P P P S G S | |
| 2116 | tcggtcagcgtgcagatcagccgcaattccaccagcccaccgatt | |
| | S V S V Q I S R N S T S P P I | |
| 2161 | aagctgccggaagacctaacggtgaccaacgccgccgacaccacc | |
| | K L P E D L T V T N A A D T T | |
| 2206 | tgcgagtag 2214 | |
| | C E * | |

The DNA sequence of the open reading frame interrupted in M. marinum mutant 86.1 and its translated protein sequence is as follows:

| | | |
|---|---|---|
| 2639 | atgcgtacttggaaagtatcgggaactgctcttgtcaccggcgtc | (SEQ. ID NO.117) |
| | M R T W K V S G T A L V T G V | (SEQ. ID NO.118) |
| 2684 | acaggccatctaggtcagcacattgcccgctggctagcgcaggcc | |
| | T G H L G Q H I A R W L A Q A | |
| 2729 | ggaaccagccatcttgtcctgctcagccgtaccgctgcagaacac | |
| | G T S H L V L L S R T A A E H | |
| 2774 | ccgcaggtagccgagttggaaaaagagctcaactccgcgggaata | |
| | P Q V A E L E K E L N S A G I | |
| 2819 | accacgacgtcgatatcggtcgatgtgaccgatcgagacgcttta | |
| | T T T S I S V D V T D R D A L | |
| 2864 | gccgccgttgtcgcccaaacacgcactgaacatggaccaatccac | |
| | A A V V A Q T R T E H G P I H | |
| 2909 | acggtcgtgcacgccgcagctcatatcgggctggtcaccactacc | |
| | T V V H A A A H I G L V T T T | |
| 2954 | gaaacaacgattgacgaattcaccaaatctttcgccgccaaagca | |
| | E T T I D E F T K S F A A K A | |
| 2999 | ctgggcgcggaaaatttgatagccgttctggaagatcagccacca | |
| | L G A E N L I A V L E D Q P P | |
| 3044 | caaacgttcatcatgttctcttcagcggcggcaacgtggggcggt | |
| | Q T F I M F S S A A A T W G G | |
| 3089 | acccgccaaggtgcatacgcggccgctaacgcttatatcgaagca | |
| | T R Q G A Y A A A N A Y I E A | |
| 3134 | ctcgtaacgcggttacgcggtcgcggttgccacgctatagcccca | |
| | L V T R L R G R G C H A I A P | |
| 3179 | gcgtggggggcctggacagacgacagaacaacatcgcaagaagtt | |
| | A W G A W T D D R T T S Q E V | |
| 3224 | gtgggatatttcagccgcatcgggcttcatcaaatatcccccgat | |
| | V G Y F S R I G L H Q I S P D | |
| 3269 | atcgccttcgccgcacttcaacaatccctcgacgtagacgacacc | |
| | I A F A A L Q Q S L D V D D T | |
| 3314 | ctgattacgatcgccgatgtcgactggagtcaattccgagacgta | |
| | L I T I A D V D W S Q F R D V | |
| 3359 | ttcaccactactggccgcgcccacaccctactggccgagctgggc | |
| | F T T T G R A H T L L A E L G | |
| 3404 | accacccaaccccagacagccgaaattcccgccatcaccgaaaac | |
| | T T Q P Q T A E I P A I T E N | |
| 3449 | tcccactacgccgcacagctagccaagcaaaccccgcagcagcaa | |
| | S H Y A A Q L A K Q T P Q Q Q | |
| 3494 | ttgacgacgctgatcgagttggtgaccactgtgactgccgcggta | |
| | L T T L I E L V T T V T A A V | |
| 3539 | ttagcgcaccccgacccggcaatgttggatcccgacctgtccttc | |
| | L A H P D P A M L D P D L S F | |
| 3584 | aaggacctcggcatcgactcgctgagcgcgctcgagctacgtaac | |
| | K D L G I D S L S A L E L R N | |
| 3629 | accttgactcgggacaccggcttgacgttgcccgcgacgctggtc | |
| | T L T R D T G L T L P A T L V | |
| 3674 | ttcgaccatcccacccctaccacagtcgctgaacatctgttggac | |
| | F D H P T P T T V A E H L L D | |
| 3719 | ctgctcagcggtgcgaccagcccgaccctggccgtcgccccgacg | |
| | L L S G A T S P T L A V A P T | |
| 3764 | caagtcgatctggatgccccggtcgcagtggtgggcatggcgtgt | |
| | Q V D L D A P V A V V G M A C | |
| 3809 | cgtttgcctggtggcatcgagtcggccgcgggtttgtgggacgtg | |
| | R L P G G I E S A A G L W D V | |
| 3854 | gtcagcaatggcatcgatgtgatgagtggctttcccaccgatcgg | |
| | V S N G I D V M S G F P T D R | |
| 3899 | ggctgggatgtggcgggactgttcgaccccgatcccgacgcagtg | |
| | G W D V A G L F D P D P D A V | |
| 3944 | ggcaagacctacacccgctacggaggatttgtggcggacgtggcc | |
| | G K T Y T R Y G G F V A D V A | |
| 3989 | ggctttgatgccgaatttttcgggatctccgcgcgagaagcaatc | |
| | G F D A E F F G I S A R E A I | |
| 4034 | acgatggatcctcaacagcgggtgctgctggaagtgtgttggcaa | |
| | T M D P Q Q R V L L E V C W Q | |
| 4079 | gcgctggaacacgcgggcatcgacccgaccaccctggaaggctcg | |
| | A L E H A G I D P T T L E G S | |
| 4124 | aacaccggagtgttcatcgggatcggggcgcagagctacgtgagt | |
| | N T G V F I G I G A Q S Y V S | |
| 4169 | gcccattccggcgttgagggttacgccctaacaggcgcctccacc | |
| | A H S G V E G Y A L T G A S T | |
| 4214 | agtgtggcctcgggccgggtggcttatgtgttggggttgcaaggc | |
| | S V A S G R V A Y V L G L Q G | |
| 4259 | ccagcaatcacggtagacaccgcatgttcgtcgtcgctggtagca | |
| | P A I T V D T A C S S S L V A | |
| 4304 | acccatctagcatgtcaatccctgcgtaacggcgaatccagcctg | |
| | T H L A C Q S L R N G E S S L | |
| 4349 | gctcttgccggtggagccacgatcatggccacacccacgccgttt | |
| | A L A G G A T I M A T P T P F | |
| 4394 | atcgagttcgctcggcaacgcggactggccgccgatggacggtgc | |
| | I E F A R Q R G L A A D G R C | |
| 4439 | aaagcgttcgcagccgccgccgatgggaccggctggggcgaaggt | |
| | K A F A A A A D G T G W G E G | |
| 4484 | gctgcagtcctggtgttggaacgtctaagcgacgcacgccgaaat | |
| | A A V L V L E R L S D A R R N | |
| 4529 | cgccatccagtacttgccgtcatcgcgggatcagcagtcaaccaa | |
| | R H P V L A V I A G S A V N Q | |
| 4574 | gacggcgcatcaaacggactgagcgcccccaacgggccagcccaa | |
| | D G A S N G L S A P N G P A Q | |
| 4619 | caacgtgttatcgctcaggcggccgccaacgcgggaattgccctg | |
| | Q R V I A Q A A A N A G I A L | |
| 4664 | gaccaggtcgatgtggtcgaagcccacggcaccggcacaaccttg | |
| | D Q V D V V E A H G T G T T L | |
| 4709 | ggtgatccgatcgaggccggcgcgctaatcgccacctacggcacc | |
| | G D P I E A G A L I A T Y G T | |
| 4754 | caccgcgatcccgagcatcccctgtggctgggatcggtgaaatcc | |
| | H R D P E H P L W L G S V K S | |
| 4799 | aacatcggacacacccaacacgcggccggcgccgccggactgatc | |
| | N I G H T Q H A A G A A G L I | |
| 4844 | aaaatgatccaagccctcaaccacgccgtcttacccgccaccctg | |
| | K M I Q A L N H A V L P A T L | |
| 4889 | cacatcgatcaacccagtccgcacatcgactggtcaaccggcacc | |
| | H I D Q P S P H I D W S T G T | |
| 4934 | gtgcaattactgaccgaggcaacgccctggcccaagactgagcat | |
| | V Q L L T E A T P W P K T E H | |
| 4979 | cttcgcaccgcagcggtttcggccttcggggtcagcggcaccaac | |
| | L R T A A V S A F G V S G T N | |
| 5024 | gcacacctgatcgtgcagcaacccccaccagaagcgccggaaacc | |
| | A H L I V Q Q P P P E A P E T | |
| 5069 | attgccgaccccgaaaccacacagcttcctcaacagcctctatta | |
| | I A D P E T T Q L P Q Q P L L | |
| 5114 | cacatttggccggtatcagcacatactcccgcagcgttgacagct | |
| | H I W P V S A H T P A A L T A | |
| 5159 | caagcacagcaacttagcgaatacctcacccaccacgaagaccta | |
| | Q A Q Q L S E Y L T H H E D L | |
| 5204 | agccttaccgatctggcccacagcctggccaccacccgtacccat | |
| | S L T D L A H S L A T T R T H | |
| 5249 | cacccctaccgcgcggctgtgaccgtacccggtgacaccgacaac | |
| | H P Y R A A V T V P G D T D N | |
| 5294 | acccgcgacgaccttctggcaggtctacactccctagccgccaac | |
| | T R D D L L A G L H S L A A N | |
| 5339 | caatcccacccaggagtgacctaccaccactaccggctaggccag | |
| | Q S H P G V T Y H H Y R L G Q | |
| 5384 | gccggtaaaacagtgttcgttttccccggccagggcagccaatac | |
| | A G K T V F V F P G Q G S Q Y | |
| 5429 | gccggcatgggcgcacagctttatcgtcaacaccccgttttcact | |
| | A G M G A Q L Y R Q H P V F T | |
| 5474 | accgctatcgatgaggtgtgcgcggcggtcgacaagcatttagat | |
| | T A I D E V C A A V D K H L D | |
| 5519 | gttccgttgcgcgaggtgatgttcaccgagccagagttgctgcag | |
| | V P L R E V M F T E P E L L Q | |
| 5564 | cagactatttatgcacaacccgcattgttcgcgttcggcgtggcc | |
| | Q T I Y A Q P A L F A F G V A | |
| 5609 | atgcacgccgtattgacccaggcaggagttaatcctgactatttg | |
| | M H A V L T Q A G V N P D Y L | |
| 5654 | ctcggtcattcagtgggagaactgaccgcagcgcatgtggctggg | |
| | L G H S V G E L T A A H V A G | |
| 5699 | gtgctttctctggaagaagccgcggtgttggtgtgcgcacggggc | |
| | V L S L E E A A V L V C A R G | |
| 5744 | cggttgatgcaaagctgcacccccggagcaatgatggccatatcg | |
| | R L M Q S C T P G A M M A I S | |
| 5789 | gccagcgagcctgccgtagccgccatgctcgaaaaccatcccgaa | |
| | A S E P A V A A M L E N H P E | |
| 5834 | gtggtcattgccgcggttaacggccccacttcagtngcaggttgc | |
| | V V I A A V N G P T S V A G C | |
| 5879 | cgggcccgctga 5890 | |
| | R A R * | |

The gene interrupted in the attenuated mutant has been characterized by sequence analysis. Using the Mycobacterium database, functional homologues of this gene have been identified in M. tuberculosis 1) [pir||G70944 emb|CAA17262.1| (AL021899) pks12 (Rv2048c) ref|NP_{—}216564.1|] 2). [pir||H70984 emb|CAB09098.1| (Z95617) pks 8 (Rv1662) ref|NP_{—}216178.1|] 3) [pir|H70621 emb|CAB06632.1| (Z85982) pks7 (Rv1661) ref|NP_{—}216177.1|] 4) [pir||H70668 emb|CAB06102.1| (ZZ83858) pks 15 (Rv2947c) ref|NP_{—}217463.1|] [pir||D70634 emb|CAB06605.1| (Z84725) pks6 (Rv0405) ref|NP_{—}214919.1|] 6) [pir||B70984 emb|CAB06093.1| (Z83857) ppsD (Rv2934) ref|NP_{—}217450.1|] 7) [pir||C70749 emb|CAA98988.1| (Z74697) ppsA (Rv2931) ref|NP_{—}217447.1|] 8) [pir||E70874 emb|CAA15929.1| (AL021070) ppsB (Rv2932) ref|NP_{—}217448.1|] 9) [pir||E70522 emb|CAB10012.1| (Z97188) pks2 (Rv3825c) ref|NP_{—}218342.1|] 10) [pir||H70819 emb|CAA17592.1| (AL022000) pks5 (Rv1527c) ref|NP_{—}216043.1|] 11) [pir||A70984 emb|CAB06099.1| (Z83857) ppsC (Rv2933) ref|NP_{—}217449.1|] 12) [pir||B70985 emb|CAB09100.1| (Z95617) pks9 (Rv1664) ref|NP_{—}216180.1|] 13) [pir||D70887 emb|CAA17864.1| (AL022076) pks13 (Rv3800c) ref|NP_{—}218317.1|] 14) [pir||D70876 emb|CAA15857.1| (AL010186) pks3 (Rv1180) ref|NP_{—}215696.1|] 15) [pir||C70984 emb|CAB06094.1| (Z83857) ppsE (Rv2935) ref|NP_{—}217451.1|. The homology with the M. tuberculosis homologue Rv2048c is 45% identity and 57% similarity; with Rv1662 is 53% identity and 64% similarity and with Rv1661 is 53% identity and 64% similarity; with Rv2947c is 66% identity and 77% similarity; with Rv0405 is 41% identity and 56% similarity; with Rv2934 is 40% identity and 55% similarity; with Rv2931 is 39% identity and 52% similarity; with Rv2932 is 42% identity and 56% similarity; with Rv3825c is 41% identity and 54% similarity; with Rv1527c is 40% identity and 56% similarity; with Rv2933 is 39% identity and 54% similarity; with Rv1664 is 39% identity and 55% similarity; with Rv3800c is 33% identity and 48% similarity; with Rv1180 is 47% identity and 60% similarity; with Rv2935 is 34% identity and 46% similarity.

This is a gene encoding a polyketide synthetase. These proteins are involved in fatty acid synthesis. That we have identified that a mutation in this gene attenuates the M. marinum strain in virulence suggests that it is required for Mycobacterium growth in the animal host.

Polyketides are lipid-like molecules that have potent biological activities. Examples of polyketides include antibiotics (erythromycin), immunosuppressants (rapamycin, FK506), antifungal agents (amphotericin B), antihelminthic agents (avermectin), and cytostatins (bafilomycin). A polyketide toxin has been recently described in Mycobacterium ulcerans (George, K. M. et al (1999). Science 283, 854-856) but no homologue was identified by sequence analysis in M. tuberculosis. Although it was recognized during analysis of the M. tuberculosis genome project that the genome contains a large number of polyketide synthesis genes, no polyketides from M. tuberculosis have been identified. That we have identified that a mutation in this gene attenuates the M. marinum strain in virulence suggests that although a polyketide toxin has not been identified, a product of this synthesis pathway is responsible for virulence. Without wishing to be bound to any mechanism, these observations suggest that a product of the polyketide synthesis pathway may be responsible for the tissue destruction and immunological modulation characteristic of diseases such as leprosy and tuberculosis.

### Example 11

### Competition Assays of Mutants

Competitive Index Assay. The wild type M. marinum ATTC 927 and mutant strains were grown in 7H9 with 10% OADC (supplemented with 50 μg/ml kanamycin for mutants) to an O.D.₆₀₀ of 1.6-1.8. The cells were harvested by centrifugation, the pellet resuspended in sterile PBS, diluted to achieve an inoculum of 1-5×10⁷ CFU/fish, and sonicated for 3 minutes at power level 3, while cooling using a cup horn accessory attached to a cell disruptor (model W-220F, Heat Systems, Ultrasonics). The inoculum was frozen in 1 ml aliquouts at −80° C. Prior to inoculation of fish for the assay, the frozen inoculum was thawed, and equal quantities of the wild type and mutant inocula combined, and the mixture sonicated as above. Three to six fish were inoculated intraperitoneally for each assay, with 0.5 ml of the mixed inocula. The number of CFU per ml of wild type and mutant strain in the mixed inocula was determined by plating on 7H10 agar with and without kanamycin (50 μg/ml) at 30° C. Fish were sacrificed after 1 week and the livers were harvested and homogenized as above. The homogenate was spun at 500×g for 5 minutes to remove organ debris. The supernatant was removed and the number of CFU per organ of both wild type and mutant strain was determined by plating on 7H10 agar with and without kanamycin (50 μg/ml) at 30° C. The competitive index (CI) was calculated using the following formula: [(CFU mutant ₒᵤₜₚᵤₜ)/(CFU wild type ₒᵤₜₚᵤₜ)]/[(CFU mutant ᵢₙₚᵤₜ)/(CFU wild type ᵢₙₚᵤₜ)].

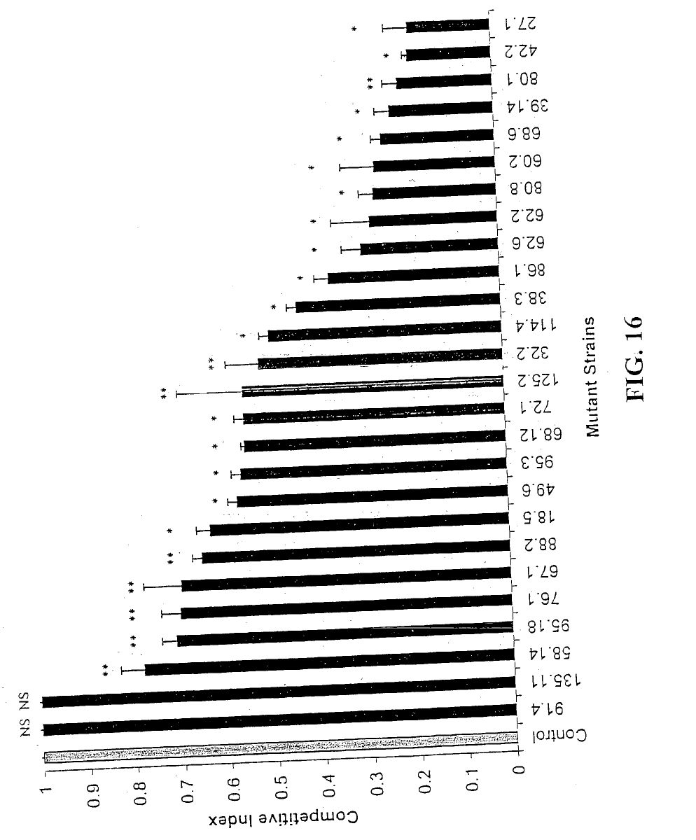

Competition assays of mutants identified by STM. The competitive index assay was used to further establish that the mutants identified by STM were attenuated in virulence. In the assay, mixed infections with mutant and wild type strains are used to provide an in vivo measure of virulence attenuation referred to as the competitive index (CI). The CI is the ratio of the mutant strain to the wild type strain in the output pool divided by the ratio of mutant strain to wild type strain in the input pool. Non-attenuated mutants have a CI=1.0, whereas, attenuated muants have a CI<1.0, with the most attenuated mutants having the lowest CI values. Of the 26 mutants tested, 24 mutants (92%) were attenuated in competition studies, with CI values ranging from 0.18 to 0.78. (See FIG. 16)

### Example 12

### Classification of M. marinum Mutants by Functional Role

Mutants identified by STM were grouped into eight classes based on the functional role of the protein encoded by the disrupted gene (See Table 1). One class of mutants belongs to the PE-PGRS and PPE family of genes that have a conserved NH₂ terminus, with large stretches of glycine-rich repeats found among the PE-PGRS gene family. Some members of this gene family have been identified as being selectively expressed in macrophages. Although the exact function of these genes is not known, they may be involved in replication in macrophages, persistence in granulomas, and antigenic diversity.

A second class of genes is involved in fatty acid synthesis. Mycobacteria are unique in that they possess an unusually complex cell wall, which is important for survival within the host environment. Mutant 114.4 is disrupted in a methoxymycolic acid synthase gene (mmaA). In pathogenic mycobacteria, mmaA genes are believed to catalyze cycloproponation of fatty acids, which possibly protect the fatty acid chain from degradation by oxygen free radicals generated within macrophages. Mutant 97.4 has a disruption in a gene homologous to an enoyl-coA hydratase that is involved in elongation of the fatty acid chains in mycobacteria.

Genes involved in aerobic metabolism make up the third category of mutants. The gene disrupted in mutant 67.1 is most homologous to adenylate succinate synthase (ADSS), a gene involved in an energy metabolism pathway. ADSS catalyzes the first step of adenosine monophosphate (AMP) biosynthesis pathway, converting AMP to IMP (inosinate monophosphate). Another mutant in this class (129.8) has a disruption in a NADPH-ferredoxin reductase homologue, which is involved in electron transport. Other genes disrupted in mutants from this class are homologous to those found in signal transduction pathways, including a methyl transferase and a dehydrogenase gene.

A fourth group of mutants (62.2, 86.1, 95.3) belongs to the polyketide synthase (PKS) family of genes. pks genes are found throughout the M. tuberculosis genome and are believed to be involved in the synthesis of cell wall lipids. A gene closely related to pks genes, the non-ribosomal peptide synthase gene (NRPS), was identified as disrupted in one of the mutants in this class. The pks and nrps genes may cooperate in biosynthesis. One class of products synthesized by the PKS/NRPS pathway in M. tuberculosis is the mycobactins, virulence factors used in iron limiting environments such as the host macrophage.

A fifth class of mutants is involved in amino acid synthesis. Two mutants are disrupted in genes homologous to cysD (27.1) and cysQ (39.2). Both cysD and cysQ are genes in the cys operon, which are required for cysteine biosynthesis, suggesting that cysteine may be of limited availability in the macrophage.

The genes disrupted in mutants 41.2 and 61.5 are classified as regulatory genes. The gene disrupted in mutant 41.2 contains an araC signature motif. Regulatory genes with these features act to both positively and negatively regulate groups of genes. The gene disrupted in mutant 61.5 is homologous to a sensor histidine kinase gene. A two-component regulatory system (devR-devS) has been shown to encode a sensor histidine kinase in M. tuberculosis.

The final group of mutants has disruptions in genes with no known function. These six mutants are highly homologous (58-85%) to hypothetical proteins in M. tuberculosis. Further characterization of these genes should lead to the identification of new mechanisms that play a role in survival of the organism in the host. Investigation of conserved motifs may give more insight into the possible function of these genes.

Four genes (mutants 49.6, 58.15/62.6, 68.6, 68.12) identified by STM that could not be grouped according to a collective function were classified as miscellaneous.

TABLE 1
Analysis of M. marinum mutants identified by STM and comparison to the M.
tuberculosis genome.

| | | M. tuberculosis | % Identity to M. | |
|---|---|---|---|---|
| Functional Class | M. marinum mutant | homologue^{∞} | tuberculosis | e value^{ψ} |
| PPE/PE | | | | |
| | 80.1 | PPE | 71 | 3.00 e⁻⁷⁷ |
| | 76.1 | PPE | 67 | 2.00 e⁻⁵¹ |
| | 32.2 | PE | 62 | 1.00 e⁻⁵² |
| | 58.14 | PPE | 62 | 3.00 e⁻⁴⁵ |
| | 42.2 | PPE | 60 | 1.00 e⁻⁷² |
| | 91.4 | PE | 59 | 1.00 e⁻⁷⁰ |
| | 60.2 | PPE | 58 | 3.4* |
| | 80.8 | PPE | 57 | 5.00 e⁻¹⁰ |
| | 135.11 | PPE | 48 | 1.00 e⁻⁵⁶ |
| fatty acid (FA) | | | | |
| synthesis | | | | |
| | 114.4 | methoxy mycolic acid | 86 | 3.00 e⁻⁸⁴ |
| | | synthase | | |
| | 39.14 | methyl transferase | 77 | 3.00 e⁻⁴⁵ |
| | 49.7 | acyl transferase | 70 | 4.00 e⁻³¹ |
| | 97.4 | enoyl-coA hydratase/ | 43 | 5.00 e⁻⁰⁸ |
| | | isomerase family | | |
| | | protein | | |
| aerobic metabolism | | | | |
| | 72.10 | L-carnitine | 84 | 9.00 e⁻⁴⁵ |
| | | dehydratase | | |
| | 62.20 | dehydrogenase | 76 | 6.00 e⁻⁴² |
| | 67.1 | ADSS | 38 | 1.9* |
| | 129.8 | NADPH-ferredoxin | 28 | 1.50 e⁺⁰⁰ |
| | | reductases | | |
| PKS | | | | |
| | 62.2 | NRPS | 51 | 2.00 e⁻³¹ |
| | 86.1 | polyketide synthase | 39 | 2.00 e⁻⁰⁸ |
| | 95.3 | polyketide synthase | 34 | 5.00 e⁻²² |
| amino acid (AA) | | | | |
| synthesis | | | | |
| | 39.2 | cysQ | 42 | 4.00 e⁻⁰⁶ |
| | 27.1 | cysD | 22 | 0.54 |
| regulatory | | | | |
| | 41.2 | araC | 73 | 2.00 e⁻²² |
| | 61.5 | sensor histidine kinase | 31 | 1.00 e⁻⁶⁰ |
| unknown | | | | |
| | 18.5 | unknown | 85 | 2.00 e⁻³⁸ |
| | 102.4/114.7 | unknown | 82 | 3.00 e⁻⁴¹ |
| | 1.4 | unknown | 65 | 9.20 e⁻⁰¹ |
| | 95.18 | unknown | 64 | 2.00 e⁻⁵³ |
| | 38.3 | unknown | 62 | 4.00 e⁻³⁴ |
| miscellaneous | | | | |
| | 58.15/62.6 | cutinase | 48 | 2.00 e⁻¹⁹ |
| | 68.6 | sporulation protein | 29 | 3.5 |
| | 68.12 | transposase | 29 | 7.00 e⁻²² |
| | 49.6 | membrane protein | 26 | 4.30 e⁺⁰⁰ |
| no M. tuberculosis | | | | |
| homolgoue | | | | |
| | 88.2 | None | N/A | 0.04 |
| | 125.20 | None | N/A | 4.00 e⁻⁰³ |
| | | [B-keto acyl synthase | | |
| | | (Streptomyces)] | | |

^{∞}Determined by comparison of sequences using BLASTX network services.
^{ψ}BLASTX e value
*Values determined from less than 300 bp of sequence (others based on 600-800 bp)
N/A: not applicable, these genes have no homologues in M. tuberciosis

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated by reference. Aspects of the invention include:

An isolated M. marinum polynucleotide comprising the sequence of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115 or 117, or a fragment or variant thereof; a polynucleotide of claim 1 which comprises a virulence gene; an avirulent M. marinum bacterium in which one or more of the above polynucleotides is mutated, so as to delete at least 50% of the coding sequence, thereby rendering the M. marinum bacterium less virulent; an avirulent M. marinum bacterium in which one or more of the above polynucleotides is mutated so as to delete at least 90% of the coding sequence, thereby rendering the M. marinum bacterium less virulent; a pharmaceutical composition, comprising an avirulent M. marinum bacterium of the invention and a pharmaceutically acceptable carrier; or an attenuated M. marinum vaccine, comprising an avirulent M. marinum bacterium of the invention, or a method for generating an avirulent M. marinum bacterium, comprising mutagenizing one or more of the polynucleotides of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109. 111, 113, 115 or 117, so as to delete at least 50% of the coding sequence.

Another aspect is an avirulent M. tuberculosis bacterium in which one or more of the following genes is mutated to render the M. tuberculosis bacterium less virulent: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c1, Rv1662, Rv1661, Rv2947c, Rv0405, Rv2934, Rv2931, Rv2932, Rv3825c, Rv1527c, Rv2933, Rv1664, Rv3800c, Rv1180 or Rv2935, wherein at least 50% of the coding sequence of the gene(s) is deleted; an avirulent M. tuberculosis bacterium of the invention, in which the mutated gene encodes a protein in the PPE/PE family, and is Rv0159c, Rv0160c, Rv0305c, Rv0355c, Rv0304c, Rv3347c, Rv1918c, Rv1753c, Rv1548c, Rv1705c, Rv2356c, Rv0160c or Rv0355c; or in which the mutated gene encodes a protein which is involved in fatty acid synthesis, and is Rv2831, Rv1428c, Rv0644c, or Rv0213c; or in which the mutated gene encodes a protein which is involved in aerobic metabolism, and is Rv3272, Rv0449c, or Rv3106; or in which the mutated gene encodes a PKS gene, and is Rv2933 or Rv0101; or in which the mutated gene encodes a protein which is involved in amino acid synthesis, and is Rv1285; or in which the mutated gene encodes a regulatory protein, and is AE006959; or in which the mutated gene is Rv3901c, Rv3234c, AE006949 or AE006933; or in which the mutated gene encodes a cutinase, a sporulation protein, a transposase or a membrane protein, and is Rv1984c, Rv3884c, Rv2339 or Rv0797; or in which one or more of the following genes is mutated to render the M. tuberculosis bacterium less virulent: Rv2048c, Rv1662 or Rv1661, wherein at least 50% of the coding sequence is deleted; or a pharmaceutical composition, comprising an avirulent M. tuberculosis bacterium of the invention and a pharmaceutically acceptable carrier; or an attenuated M. tuberculosis vaccine, comprising an avirulent M. tuberculosis bacterium of the invention.

Another aspect is a method to elicit an immune response in a patient in need thereof, comprising administering to said patient an avirulent M. tuberculosis bacterium of the invention; or a method for generating an avirulent M. tuberculosis bacterium, comprising mutagenizing one or more of the following M. tuberculosis genes so as to delete at least 50% of the coding sequence: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c, Rv1662, Rv1661, Rv2947c, Rv0405, Rv2934, Rv2931, Rv2932, Rv3825c, Rv1527c, Rv2933, Rv1664, Rv3800c, Rv1180 or Rv2935, or a method to identify an agent which reduces the ability of an M. tuberculosis bacterium to survive in a host, comprising determining whether the agent disrupts expression of one of the following M. tuberculosis genes: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c1, Rv1662, Rv1661, Rv2947c, Rv0405, Rv2934, Rv2931, Rv2932, Rv3825c, Rv1527c, Rv2933, Rv1664, Rv3800c, Rv1180 or Rv2935; wherein the method comprises a) overexpressing one of said M. tuberculosis genes in a heterologous bacterium, b) exposing said bacterium overexpressing said gene to a putative agent, and c) determining if the agent reduces the viability or growth of a wild type bacterium, but not the bacterium which overexpresses said gene; or wherein the method comprises a) expressing a reporter gene under the control of a promoter which regulates one of said M. tuberculosis genes, in a heterologous bacterium, b) exposing said bacterium expressing said reporter gene to a putative agent, and c) determining if the agent selectively inhibits expression of the reporter gene.

Another aspect is a method to test for the presence of a M. tuberculosis infection in a subject, comprising administering to the subject one or more M. tuberculosis proteins encoded by the following genes: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c1, Rv1662, Rv1661, Rv2947c, Rv0405, Rv2934, Rv2931, Rv2932, Rv3825c, Rv1527c, Rv2933, Rv1664, Rv3800c, Rv1180 or Rv2935, and determining if cell-mediated immunity is induced. Another aspect is an avirulent M. tuberculosis bacterium of the invention, which further comprises a heterologous gene and serves as a carrier to express said heterologous gene.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding PCT, WO01/19993 and U.S. Provisional Application Serial No. 60/367,206 filed Mar. 26, 2002 and No. 60/366,262, Mar. 22, 2002, are incorporated by reference herein in their entireties.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. An isolated M. marinum polynucleotide comprising the sequence of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115 or 117, or a fragment thereof.

2. A polynucleotide of claim 1 which comprises a virulence gene.

3. An avirulent M. marinum bacterium in which one or more of the polynucleotides 59, 67, 71, 73, 75, 79, 81, 83, 85, 87, 89, 91, 93, 95, 97, 99, 101, 103, 105, 107 or 109 of claim 1 are mutated so as to delete at least 50% of the coding sequence, thereby rendering the M. marinum bacterium less virulent.

4. An avirulent M. marinum bacterium in which one or more of the polynucleotides 117, 51, 53, 57 or 61 of claim 1 are mutated so as to delete at least 50% of the coding sequence, thereby rendering the M. marinum bacterium less virulent.

5. An avirulent M. marinum bacterium in which one or more of the polynucleotides 115, 117, 111, 57, 55, 65, 51, 53, 63, 69 or 77 of claim 1 are mutated so as to delete at least 50% of the coding sequence, thereby rendering the M. marinum bacterium less virulent.

6. An avirulent M. marinum bacterium in which one or more of the polynucleotides of claim 1 is mutated so as to delete at least 90% of the coding sequence, thereby rendering the M. marinum bacterium less virulent.

7. A pharmaceutical composition, comprising an avirulent M. marinum bacterium of claim 3 and a pharmaceutically acceptable carrier.

8. An attenuated M. marinum vaccine, comprising an avirulent M. marinum bacterium of claim 3.

9. A method for generating an avirulent M. marinum bacterium, comprising mutagenizing one or more of the polynucleotides of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115 or 117, so as to delete at least 50% of the coding sequence, hereby rendering the M. marinum bacterium less virulent.

10. An avirulent M. tuberculosis bacterium in which one or more of the following genes is mutated so as to delete at least 50% of the coding sequence, to render the M. tuberculosis bacterium less virulent: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c1, Rv1662, Rv1661, Rv2947c, Rv0405, Rv2934, Rv2931, Rv2932, Rv3825c, Rv1527c, Rv2933, Rv1664, Rv3800c, Rv1180 or Rv2935, wherein at least 50% of the coding sequence of the gene(s) is deleted.

11. The avirulent M. tuberculosis bacterium of claim 10, in which the mutated gene encodes a protein in the PPE/PE family, and is Rv0159c, Rv0160c, Rv0305c, Rv0355c, Rv0304c, Rv3347c, Rv1918c, Rv1753c, Rv1548c, Rv1705c, Rv2356c, Rv0160c or Rv0355c.

12. The avirulent M. tuberculosis bacterium of claim 10, in which the mutated gene encodes a protein which is involved in fatty acid synthesis, and is Rv2831, Rv1428c, Rv0644c, or Rv0213c.

13. The avirulent M. tuberculosis bacterium of claim 10, in which the mutated gene encodes a protein which is involved in aerobic metabolism, and is Rv3272, Rv0449c, or Rv3106.

14. The avirulent M. tuberculosis bacterium of claim 10, in which the mutated gene encodes a PKS gene, and is Rv2933 or Rv0101.

15. The avirulent M. tuberculosis bacterium of claim 10, in which the mutated gene encodes a protein which is involved in amino acid synthesis, and is Rv1285.

16. The avirulent M. tuberculosis bacterium of claim 10, in which the mutated gene encodes a regulatory protein, and is AE006959.

17. The avirulent M. tuberculosis bacterium of claim 10, in which the mutated gene is Rv3901c, Rv3234c, AE006949, or AE006933.

18 The avirulent M. tuberculosis bacterium of claim 10, in which the mutated gene encodes a cutinase, a sporulation protein, a transposase or a membrane protein, and is Rv1984c, Rv3884c, Rv2339 or Rv0797.

19. An avirulent M. tuberculosis bacterium in which one or more of the following genes is mutated so as to delete at least 50% of the coding sequence, to render the M. tuberculosis bacterium less virulent: Rv2048c, Rv1662, Rv1661, Rv0159c, Rv0160c, Rv0305c, Rv0355c, Rv0304c, Rv3347c, Rv1918c, Rv1753c, Rv1984c, Rv3452, Rv3451, Rv2947c, Rv0405, Rv2934, Rv2931, Rv2932, Rv3825c, Rv1527c, Rv2933, Rv1664, Rv3800c, Rv1180 or Rv2935, wherein at least 50% of the coding sequence is deleted.

20. An avirulent M. tuberculosis bacterium in which one or more of the following genes is mutated so as to delete at least 50% of the coding sequence, to render the M. tuberculosis bacterium less virulent: Rv0101, Rv1285, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106 or Rv3347c, wherein at least 50% of the coding sequence of the gene(s) is deleted.

21. A pharmaceutical composition, comprising an avirulent M. tuberculosis bacterium of claim 10 and a pharmaceutically acceptable carrier.

22. An attenuated M. tuberculosis vaccine, comprising an avirulent M. tuberculosis bacterium of claim 10.

23. A method to elicit an immune response in a patient in need thereof, comprising administering to said patient an avirulent M. tuberculosis bacterium of claim 10.

24. A method for generating an avirulent M. tuberculosis bacterium, comprising mutagenizing one or more of the following M. tuberculosis genes so as to delete at least 50% of the coding sequence: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv1984c, Rv3452, Rv3451, Rv2048c1, Rv1662, Rv1661, Rv2947c, Rv0405, Rv2934, Rv2931, Rv2932, Rv3825c, Rv1527c, Rv2933, Rv1664, Rv3800c, Rv1180 or Rv2935.

25. A method to identify an agent which reduces the ability of an M. tuberculosis bacterium to survive in a host, comprising determining whether the agent disrupts expression of one of the following M. tuberculosis genes: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv3901c, Rv3234c, Rv1705c, Rv2933, AE006949, Rv3272, Rv2356c, Rv1428c, AE006959, Rv0644c, Rv2339, Rv0160c, Rv0355c, Rv0213c, AE006933, Rv0449c, Rv0797, Rv3106, Rv3347c, Rv]984c, Rv3452, Rv3451, Rv2048c1, Rv1662, Rv1661, Rv2947c, Rv0405, Rv2934, Rv2931, Rv2932, Rv3825c, Rv1527c, Rv2933, Rv1664, Rv3800c, Rv1180 or Rv2935.

26. The method of claim 25, comprising
a) overexpressing one of said M. tuberculosis genes in a heterologous bacterium,
b) exposing said bacterium overexpressing said gene to a putative agent, and
c) determining if the agent reduces the viability or growth of a wild type bacterium, but not the bacterium which overexpresses said gene.

27. The method of claim 25, comprising
a) expressing a reporter gene under the control of a promoter which regulates one of said M. tuberculosis genes, in a heterologous bacterium,
b) exposing said bacterium expressing said reporter gene to a putative agent, and
c) determining if the agent selectively inhibits expression of the reporter gene.

28. A method to test for the presence of a M. tuberculosis infection in a subject, comprising administering to the subject one or more M. tuberculosis proteins encoded by the following genes: Rv0159c, Rv0160c, Rv0305c, Rv0355c, Rv0304c, Rv3347c, Rv0101, Rv1918c, Rv1753c, Rv1285, Rv1984c, Rv3452, Rv3451, Rv3884c, Rv1548c, Rv2831, Rv2348c, Rv3901c, Rv3234c, Rv1705c, Rv2933, NC_{—}002755, AE006949, Rv3272, Rv2356c, Rv1428c, AE006959, Rv0644c, mmaA2, Rv2339, Rv0160c, Rv0355c, Rv0213c, AE006933, Rv0449c, Rv0797, Rv3106 or Rv3347c and determining if cell-mediated immunity is induced.

29. An avirulent M. tuberculosis bacterium of claim 10, which further comprises a heterologous gene and serves as a carrier to express said heterologous gene.
